(19) United States
(12) Patent Application Publication
(10) Pub. No.: US 2023/0183682 A1
(43) Pub. Date: Jun. 15, 2023

(54) Preparation of RNA and DNA Sequencing Libraries Using Bead-Linked Transposomes

(71) Applicants: Illumina, Inc., San Diego, CA (US); Illumina Cambridge Limited, Cambridge (GB)

(72) Inventors: Niall Anthony Gormley, Cambridge (GB); Andrew B. Kennedy, San Diego, CA (US); Robert Scott Kuersten, Madison, WI (US); Gary Schroth, Danville, CA (US); Carlo Randise-Hinchliff, San Diego, CA (US); Sarah E. Shultzaberger, San Diego, CA (US); Fiona Kaper, San Diego, CA (US); Yir-Shyuan Wu, Albany, CA (US); Tarun Khurana, Fremont, CA (US); Foad Mashayekhi, San Diego, CA (US)

(73) Assignees: Illumina, Inc., San Diego, CA (US); Illumina Cambridge Limited, Cambridge (GB)

(21) Appl. No.: 18/163,405

(22) Filed: Feb. 2, 2023

Related U.S. Application Data
(63) Continuation of application No. PCT/US21/44715, filed on Aug. 5, 2021.
(60) Provisional application No. 63/061,885, filed on Aug. 6, 2020.
(60) Provisional application No. 63/165,830, filed on Mar. 25, 2021.
(60) Provisional application No. 63/168,802, filed on Mar. 31, 2021.
(60) Provisional application No. 63/219,014, filed on Jul. 7, 2021.

(51) Int. Cl. C12N 15/10

(52) U.S. Cl. CPC C12N 15/1065

(57) ABSTRACT

This application describes methods of preparing an immobilized library of tagged RNA fragments. Also described herein are a number of methods of preparing DNA and RNA sequencing libraries from a single sample. These methods can include library preparation from single cells.

### CROSS-REFERENCE TO RELATED APPLICATIONS

This application is a bypass continuation of PCT/US2021/044715, filed Aug. 5, 2021, which claims the benefit of priority of US Provisional Application No. 63/061,885, filed Aug. 6, 2020; US Provisional Application No. 63/165,830, filed Mar. 25, 2021, US Provisional Application No. 63/168,802, filed Mar. 31, 2021; and US Provisional Application No. 63/219,014, filed Jul. 7, 2021, the contents of which are each incorporated by reference herein in their entireties for any purpose.

### DESCRIPTION

### Field

This application relates to preparation of RNA sequencing libraries using bead-linked transposomes. Methods of preparing RNA and DNA sequencing libraries from a single sample are also described.

### Background

RNA sequencing is important for a number of uses. For example, sequencing of full RNA transcripts allows for studying any variation in a transcript, such as differences in splicing. Also, RNA sequencing can be used to perform a transcript count and count the number of transcripts of various genes.

RNA sequencing (RNA-seq) profiles the transcriptome using next generation sequencing. Common technique involves converting single-strand RNA into single or double stranded cDNA fragments. Adapters are then added to the ends of each fragment in library preparation, which is required for many sequencing platforms. Depending on the approach used, the information pertaining to which strand the RNA originates from can be retained. This is known as strand-specific RNA-seq, and it improves on standard approaches by accurately identifying antisense transcripts, determining the strand of non-coding RNAs (e.g. LncRNA), and demarcating boundaries of overlapping genes. Further, strand-specific RNA-seq is a desirable approach as it provides a more accurate estimate of transcript expression.

Many current protocols for sequencing RNA samples employ a sample preparation method that converts the RNA in the sample into a double-stranded cDNA format prior to sequencing. Methods that improve workflow and ease-of-use are needed for RNA sequencing, such as methods that allow preparation of strand-specific RNA libraries using tagmentation.

Further, current single-cell RNA sequencing analysis relies on unique molecular identifier (UMI)-based methods to ensure quantitative measurements of expression. This approach is especially relevant to single-cell RNA sequencing which often relies on significant levels of cDNA pre-amplification to obtain enough input for sequencing. Amplification bias can be corrected by collapsing reads with matching UMIs and mapping sites. However, UMI-based methods provide reads coming only from the 3′ of transcripts, where the UMI is attached in most current protocols, such as CEL-seq2 (Hashimshony T et al. Genome Biol. 17:77 (2016)), Drop-Seq (Macosko EZ, et al. Cell 161:1202-14 (2015)), Smart-seq2 (Picelli S, et al. Nature Protocols 9:171-181 (2014)) or a UMI can also be attached at the 5′ end, such as in STRT-seq (Islam S, et al. Nat Methods 2013;11:163-6). Thus, while alternative splicing studies have become a prominent application of RNA sequencing, single-cell level measurements of isoform expression have remained limited, as there are very few technologies available that can enable single cell isoform expression analysis. Methods that improve on cDNA production from RNA thus can have use in a wide variety of different applications.

Transposomes bound to a surface (such as a bead) can tagment long molecules of double-stranded (ds) DNA and produce template libraries on beads or other surfaces (See U.S. Pat. No. 9,683,230). Anchoring transposomes to beads can help control insert size and yield during tagmentation and is the basis of the Illumina DNA Flex PCR-Free (research use only, RUO) technology, previously known as Illumina's Nextera technology. In these methods, the Tn5 enzyme catalyzes the translocation of adapters required for sequencing to the ends of double-stranded DNA or cDNA through a “cut-and-paste” mechanism referred as tagmentation. In library preparation, tagmentation is either done in solution or on magnetic beads using bead-linked transposomes (BLTs). Tagmentation based methods require fewer steps, produces higher conversion efficiencies and are more accurate than ligation-based methods. However, there are no strand-specific RNA-seq methods available with tagmentation methods.

Further, asymmetrical tagmentation methods, such as Illumina DNA Flex PCR-Free, include tagmentation with BLTs that contain a mixture of A14 and B15 transposomes, wherein A14 and B15 comprise sequencing adapter sequences. Fragments that are tagmented with only A14 or only B15 (i.e., fragments with a A14 sequence at both ends of fragments or a B15 sequence at both ends of fragments) do not make a viable library product, because standard Illumina SBS sequencing methods require the presence of A14 at one end of fragments and B15 at the other end. Accordingly, roughly half of all tagmented fragments are lost leading to reduced library preparation efficiency with asymmetrical tagmentation methods in the prior art. Methods that avoid such loss of tagmentation products, such as by incorporating symmetrical tagmentation protocols wherein all transposome complexes comprise identical transposomes, are needed to improve yield with methods including tagmentation.

Transposomes can also tagment an ‘apparent’ DNA:RNA duplex. For example, a polyT capture oligo can be bound to a flow-cell surface and capture mRNA transcripts via their 3′ polyA tail, followed by treatment with an reverse transcriptase (RT) enzyme to generate a duplex comprising a ‘first strand synthesis’ cDNA strand bound to the original RNA transcript. A transposome from solution can then generate templates that are clusterable and sequencable, as described in WO 2013131962A1. Next, transposome to the solution could be added to generate templates that were clusterable and sequencable, without generating the 2^{nd} strand cDNA and hence the double-stranded cDNA. This suggested that the transposomes can tagment an ‘apparent’ RNA/DNA duplex. The hypothesis for this mechanism is that the reverse transcriptase initiates second stand synthesis via nicks that occur in the RNA strand and that these dsDNA duplexes that are the substrates for the transposomes. However, as a consequence of using transposomes from solution, reads are only generated from the 3′ end of the transcripts (as shown in FIGS. 1B and 1C). This application describes methods of tagmentation on RNA that avoid 3′ bias.

Further, this application describes means to prepare RNA and DNA sequencing libraries from the same sample. Many sequencing-based assays benefit from being able to characterize both DNA and RNA content of a sample “multi-omic” analyses. Current sample preparation/sequencing workflows to analyze total nucleic acid (TNA, comprising both DNA and RNA) from a sample are limited for multi-omics, because these workflows are either cumbersome and/or have significant loss of sample (e.g., requires splitting a TNA sample into two biomolecule- specific library preparations) or do not distinguish biomolecule type (e.g., a next-generation sequencing (NGS) read can either be from an RNA or DNA molecule). The present disclosure describes various methodologies for efficient TNA sample preparation for NGS, using methods that identify the originating biomolecule type (RNA or DNA). As such, these methodologies can allow simplified multi-omic library preparation by tagmentation of double-stranded nucleic acids.

Described herein are also methods of preparing RNA libraries that incorporate 3′ unique molecular identifiers (UMIs) and methods of preparing strand-specific RNA libraries with tagmentation.

### SUMMARY

Described herein are methods of preparing RNA and DNA libraries.

Embodiment 1. A method of preparing an immobilized library of tagged DNA:RNA fragments from target RNA comprising (a) applying a sample comprising target RNA to a solid support having transposome complexes and capture oligonucleotides immobilized thereon, wherein the transposome complexes comprise a transposase bound to a first polynucleotide comprising a 3′ portion comprising a transposon end sequence and a first tag; wherein the sample is applied to the solid support under conditions wherein the 3′ end of the target RNA binds to the capture oligonucleotides; (b) adding a reverse transcriptase polymerase under conditions to synthesize cDNA and generate immobilized DNA:RNA duplexes on the capture oligonucleotides; and (c) performing tagmentation on the DNA:RNA duplexes with the transposome complexes under conditions wherein the DNA:RNA duplexes are tagged on the 5′ end of one strand, thereby producing an immobilized library of DNA:RNA fragments wherein at least one strand is 5′-tagged with the first tag.

Embodiment 2. The method of embodiment 1, wherein the transposome complexes are reversibly deactivated before performing tagmentation and performing tagmentation comprises activating the transposome complexes.

Embodiment 3. The method of embodiment 2, wherein the transposome complexes are reversibly deactivated by a transposome deactivator bound to the transposome complex.

Embodiment 4. The method of embodiment 3, wherein the transposome deactivator is bound to a Tn5 binding site of the transposome complex.

Embodiment 5. The method of embodiment 3 or embodiment 4, wherein the transposome deactivator comprises dephosphorylated ME′, extra bases, inhibitor duplexes, and/or heat-labile antibodies.

Embodiment 6. The method of any one of embodiments 2 to 5, wherein the transposome complex is activated in step (c) by removing the transposome deactivator.

Embodiment 7. The method of any one of embodiments 1-6, wherein the capture oligonucleotides comprise a polyT sequence.

Embodiment 8. The method of any one of embodiments 1-7, wherein the target RNA comprises a sequence complementary to at least a portion of one or more of the capture oligonucleotides.

Embodiment 9. The method of any one of embodiments 1-8, wherein the transposome complex is immobilized to the solid support via the first polynucleotide.

Embodiment 10. The method of any one of embodiments 1-9, wherein the transposome complexes comprise a second polynucleotide comprising a region complementary to the transposon end sequence.

Embodiment 11. The method of embodiment 10, wherein the transposome complex is immobilized to the solid support via the second polynucleotide.

Embodiment 12. The method of any one of embodiments 1-11, further comprising washing the solid support after step (a) to remove any unbound target RNA.

Embodiment 13. The method of any one of embodiments 1-12, wherein the transposome complexes are present on the solid support at a density of at least 10³, 10⁴, 10⁵, or 10⁶ complexes per mm².

Embodiment 14. The method of any one of embodiments 1-13, wherein the transposase comprises a Tn5 transposase.

Embodiment 15. The method of embodiment 14, wherein the Tn5 transposase is hyperactive Tn5 transposase.

Embodiment 16. The method of any one of embodiments 1-15, wherein the lengths of the double-stranded fragments in the immobilized library are adjusted by increasing or decreasing the density of transposome complexes on the solid support.

Embodiment 17. The method of any one of embodiments 1-16, wherein at least 50%, 55%, 60%, 65%, 70%, 75%, 80%, 85%, 90%, 95%, 96%, 97%, 98%, or 99% of the first tags comprise the same tag domain.

Embodiment 18. The method of any one of embodiments 1-17, wherein the tag comprises a region for cluster amplification.

Embodiment 19. The method of any one of embodiments 1-18, wherein the tag comprises a region for priming a sequencing reaction.

Embodiment 20. The method of any one of embodiments 1-19, wherein the solid support comprises microparticles, beads, a planar support, a patterned surface, or wells.

Embodiment 21. The method of embodiment 20, wherein the planar support is an inner or outer surface of a tube.

Embodiment 22. The method of any one of embodiments 1-21, wherein performing tagmentation produces double stranded DNA:RNA duplexes bridged to two immobilized transposome complexes on the solid support, optionally wherein a second strand of DNA is synthesized to prepare double stranded DNA before performing tagmentation.

Embodiment 23. The method of embodiment 22, wherein the length of the bridged duplexes is from 100 base pairs to 1500 base pairs.

Embodiment 24. The method of any one of embodiments 1-23, wherein the sample that is applied to the solid support is blood.

Embodiment 25. The method of any one of embodiments 1-24, wherein the sample that is applied to the solid support is a cell lysate.

Embodiment 26. The method of embodiment 25, wherein the cell lysate is a crude cell lysate.

Embodiment 27. The method of any one of embodiments 1-26, wherein the sample that is applied to the solid support has a 260/280 absorbance ratio that is less than or equal to 1.7.

Embodiment 28. The method of any one of embodiments 1-27, further comprising lysing cells in the sample after applying the sample to the solid support.

Embodiment 29. The method of any one of embodiments 1-28, further comprising: (d) contacting solution-phase transposome complexes with the immobilized DNA:RNA fragments under conditions whereby the DNA:RNA fragments are further fragmented by the solution-phase transposome complexes; thereby obtaining immobilized nucleic acid fragments having one end in solution.

Embodiment 30. The method of embodiment 29, wherein the solution-phase transposome complexes comprise a second tag, thereby generating immobilized nucleic acid fragments having a second tag in solution.

Embodiment 31. The method of embodiment 30, wherein the first and second tags are different.

Embodiment 32. The method of any one of embodiments 29 to 31, wherein at least 50%, 55%, 60%, 65%, 70%, 75%, 80%, 85%, 90%, 95%, 96%, 97%, 98%, or 99% of the solution- phase transposome complexes comprise a second tag.

Embodiment 33. The method of any one of embodiments 29 to 32, further comprising amplifying the fragments on the solid support by reacting a polymerase and an amplification primer corresponding to a portion of the first polynucleotide.

Embodiment 34. A solid support having a library of tagged RNA fragments immobilized thereon prepared according to the method of any one of embodiments 1-33.

Embodiment 35. A method of preparing an immobilized library of tagged DNA:RNA fragments from target RNA comprising (a) applying a sample comprising target RNA to a solid support having capture oligonucleotides and a first polynucleotide immobilized thereon, wherein the first polynucleotide comprises a 3′ portion comprising a transposon end sequence, and a first tag wherein the sample is applied to the solid support under conditions wherein the 3′ end of the target RNA binds to the capture oligonucleotides; (b) adding a transposase under conditions wherein the transposase binds to the first polynucleotide to form a transposome complex; (c) adding a reverse transcriptase polymerase under conditions to synthesize cDNA and generate immobilized DNA:RNA duplexes on the capture oligonucleotides; and (d) performing tagmentation on the DNA:RNA duplexes with the transposome complexes under conditions wherein the DNA:RNA duplexes are tagged on the 5′ end of one strand, thereby producing an immobilized library of DNA:RNA fragments wherein at least one strand is 5′-tagged with the first tag.

Embodiment 36. A method of preparing an immobilized library of tagged DNA:RNA fragments from target RNA comprising (a) applying a sample comprising target RNA to a solid support having capture oligonucleotides and a first polynucleotide immobilized thereon, wherein the first polynucleotide comprises a 3′ portion comprising a transposon end sequence, and a first tag wherein the sample is applied to the solid support under conditions wherein the 3′ end of the target RNA binds to the capture oligonucleotides; (b) adding a reverse transcriptase polymerase under conditions to synthesize cDNA and generate immobilized DNA:RNA duplexes on the capture oligonucleotides; (c) adding a transposase under conditions wherein the transposase binds to the first polynucleotide to form a transposome complex; and (d) performing tagmentation on the DNA:RNA duplexes with the transposome complexes under conditions wherein the DNA:RNA duplexes are tagged on the 5′ end of one strand, thereby producing an immobilized library of DNA:RNA fragments wherein at least one strand is 5′-tagged with the first tag.

Embodiment 37. The method of any one of embodiments 35 or 36, wherein the applying a sample comprising target RNA to a solid support is performed in a droplet.

Embodiment 38. The method of embodiment 37, wherein the applying a sample comprising target RNA to a solid support comprises (a) providing a single cell in a droplet together with a bead; (b) lysing the cell in the droplet; (c) releasing the target RNA from the single cell; and (d) capturing the target RNA on the bead.

Embodiment 39. The method of any one of embodiments 37-38, wherein the droplet is removed before synthesizing cDNA.

Embodiment 40. The method of any one of embodiments 35-39, further comprising delivering the immobilized library of DNA:RNA fragments on the solid support to a surface for sequencing.

Embodiment 41. The method of embodiment 40, further comprising after the delivering, (a) capturing the solid support with the immobilized library of DNA:RNA fragments on the surface for sequencing; (b) releasing the immobilized fragments from the solid support; and (c) capturing the fragments on the surface for sequencing.

Embodiment 42. The method of embodiment 41, further comprising sequencing the fragments on the surface for sequencing.

Embodiment 43. The method of embodiment 42, wherein the surface for sequencing is a flowcell.

Embodiment 44. The method of any one of embodiments 35-43, wherein the applying a sample comprising target RNA to a solid support is performed in a microwell on the solid support.

Embodiment 45. The method of embodiment 44, the applying a sample comprising target RNA to a solid support comprises lysing a cell and releasing target RNA from the single cell in a microwell.

Embodiment 46. The method of any one of embodiments 35-45, further comprising releasing the immobilized library of DNA:RNA fragments and sequencing the fragments in the same microwell.

Embodiment 47. The method of any one of embodiments 44-46, wherein the solid support is a flowcell comprising microwells.

Embodiment 48. The method of embodiment 47, wherein the sequencing data allows for the resolution of fragments that had been immobilized on the same solid support based on the spatial proximity of fragments on the surface for sequencing.

Embodiment 49. The method of any one of embodiments 1-48, wherein performing tagmentation on the DNA:RNA duplexes with the transposome complexes is performed with two different transposome complexes, wherein the different transposome complexes comprise first transposons comprising different adapter sequences.

Embodiment 50. The method of embodiment 49, wherein at least some fragments are tagged with a first-read sequence adapter sequence at the 5′ end of one strand and with a second- read sequence adapter sequence at the 5′ end of the other strand.

Embodiment 51. The method of any one of embodiments 1-48, wherein performing tagmentation on the DNA:RNA duplexes with the transposome complexes is performed with transposome complexes comprising first transposons comprising the same adapter sequence.

Embodiment 52. The method of embodiment 51, wherein all the transposome complexes are identical.

Embodiment 53. The method of embodiment 51 or 52, wherein the fragments are tagged with the same adapter sequence at the 5′ end of both strands of the double-stranded fragments.

Embodiment 54. The method of embodiment any one of embodiments 51-53, further comprising (a) releasing the double-stranded target nucleic acid fragments from the transposome complex, (b) hybridizing a polynucleotide comprising an adapter sequence, a UMI, and a sequence all or partially complementary to the first 3′ end transposon sequence, wherein the adapter sequence comprised in the polynucleotide is different from the adapter sequence comprised in the transposome complexes, (c) optionally extending a second strand of the double-stranded target nucleic acid fragments, (d) optionally ligating the polynucleotide or extended polynucleotide with the double-stranded target nucleic acid fragments, and (e) producing double-stranded target nucleic acid fragments comprising the UMI, wherein the UMI is located directly adjacent to the 3′ end of the insert DNA.

Embodiment 55. The method of any one of embodiments 51-53, further comprising (a) releasing the double-stranded target nucleic acid fragments from the transposome complex, (b) hybridizing a first polynucleotide comprising a UMI and an adapter sequence, wherein the adapter in the first transposon is different from the adapter in the first polynucleotide, (c) optionally adding a second polynucleotide comprising regions complementary to the first polynucleotide to produce a double-stranded adapter, (d) optionally extending a second strand of the double-stranded target nucleic acid fragments, (e) optionally ligating the double-stranded adapter with the double-stranded target nucleic acid fragments, and (f) producing double stranded target nucleic acid fragments comprising a UMI, wherein the UMI is located between the double-stranded target nucleic acid fragments and the adapter sequence from the first polynucleotide.

Embodiment 56. The method of embodiment 54 or embodiment 55, wherein fragments are tagged with a first-read sequence adapter sequence from the first transposon at the 5′ end of one strand and with a second-read sequence adapter sequence from the first polynucleotide at the 5′ end of the other strand.

Embodiment 57. A method of preparing an immobilized library of tagged DNA:RNA fragments from target RNA comprising (a) applying a sample comprising target RNA to a solid support having capture oligonucleotides immobilized thereon; (b) adding a reverse transcriptase polymerase under conditions to synthesize cDNA and generate immobilized DNA:RNA duplexes on the capture oligonucleotides; (c) performing tagmentation on the DNA:RNA duplexes with the transposome complexes in solution under conditions wherein the DNA:RNA duplexes are tagged on the 5′ end of one strand, thereby producing an immobilized library of DNA:RNA fragments wherein at least one strand is 5′- tagged with the first tag.

Embodiment 58. The method of embodiment 57, wherein the RNA is mRNA, and the capture oligonucleotide comprises a polyT sequence.

Embodiment 59. The method of embodiment 58, wherein the library of fragments comprises DNA:RNA fragments generated from the 3′ end of one or more RNA.

Embodiment 60. The method of any one of embodiments 57-59, wherein the capture oligonucleotide further comprises a first-read sequencing adapter sequence, bead code, and/or one or more additional adapter sequences.

Embodiment 61. The method of any one of embodiments 57-60, wherein the transposomes complexes in solution comprise a first transposome comprising a second-read sequence adapter sequence and/or one or more additional adapter sequences.

Embodiment 62. The method of any one of embodiments 57-61, wherein the library of DNA:RNA fragments are sequenced without amplifying fragments before sequencing.

Embodiment 63. A solid support comprising capture oligonucleotides and a first polynucleotide immobilized thereon, wherein the first polynucleotide comprises a 3′ portion comprising a transposon end sequence, and a first tag.

Embodiment 64. The solid support of embodiment 63, wherein the solid support is a bead.

Embodiment 65. The solid support of embodiment 64, wherein the first polynucleotide further comprises a bead code.

Embodiment 66. The solid support of embodiment 65, wherein the bead is comprised in a pool of beads, wherein each bead comprises an immobilized first polynucleotide comprising a different bead code as compared to the bead code comprised in other beads in the pool.

Embodiment 67. The solid support of any one of embodiments 63-66, further comprising a transposase bound to the first polynucleotide to form a transposome complex.

Embodiment 68. The solid support of embodiment 67, wherein the transposome complex is reversibly deactivated.

Embodiment 69. The solid support of 68, wherein the transposome complex is reversibly deactivated by a transposome deactivator bound to the transposome complex.

Embodiment 70. The solid support of embodiment 69, wherein the transposome deactivator is bound to a Tn5 binding site of the transposome complex.

Embodiment 71. The solid support of embodiment 69 or embodiment 70, wherein the transposome deactivator comprises dephosphorylated ME′, extra bases, inhibitor duplexes, and/or heat-labile antibodies.

Embodiment 72. A solid support comprising capture oligonucleotides and an immobilized oligonucleotide, wherein the immobilized oligonucleotide comprises a sequence for hybridizing to a hybridization sequence comprised in a second transposon comprised in a transposome complex.

Embodiment 73. The solid support of embodiment 72, wherein the solid support is a bead.

Embodiment 74. The solid support of embodiment 73, wherein the immobilized oligonucleotide further comprises a bead code and/or one or more adapter sequence.

Embodiment 75. The solid support of embodiment 74, wherein the bead is comprised in a pool of beads, wherein each bead comprises an immobilized oligonucleotide comprising a different bead code as compared to the bead code comprised in immobilized oligonucleotides comprised in other beads in the pool.

Embodiment 76. The solid support of any one of embodiments 63 to 75, wherein the capture oligonucleotides comprise a polyT sequence.

Embodiment 77. The solid support of any one of embodiments 63 to 76, wherein the capture oligonucleotides comprise a sequence complementary to at least a portion of the target RNA.

Embodiment 78. The solid support of any one of embodiments 63 to 77, wherein the transposome complex is immobilized to the solid support via the first polynucleotide.

Embodiment 79. The solid support of any one of embodiments 63 to 78, wherein the transposome complex comprises a second polynucleotide comprising a region complementary to the transposon end sequence.

Embodiment 80. The method of embodiment 79, wherein the transposome complex is immobilized to the solid support via the second polynucleotide.

Embodiment 81. The solid support of any one of embodiments 63 to 80, wherein the transposome complexes are present on the solid support at a density of at least 10³, 10⁴, 10⁵, or 10⁶ complexes per mm².

Embodiment 82. The solid support of any one of embodiments 63 to 81, wherein the transposase comprises a Tn5 transposase.

Embodiment 83. The method of embodiment 82, wherein the Tn5 transposase is hyperactive Tn5 transposase.

Embodiment 84. The solid support of any one of embodiments 63 to 83, wherein at least 50%, 55%, 60%, 65%, 70%, 75%, 80%, 85%, 90%, 95%, 96%, 97%, 98%, or 99% of the first tags comprise the same tag domain.

Embodiment 85. The solid support of any one of embodiments 63 to 84, wherein the tag comprises a region for cluster amplification.

Embodiment 86. The solid support of any one of embodiments 63 to 85, wherein the tag comprises a region for priming a sequencing reaction.

Embodiment 87. The solid support of any one of embodiments 63 to 86, wherein the solid support comprises microparticles, beads, a planar support, a patterned surface, or wells.

Embodiment 88. The solid support of embodiment 87, wherein the planar support is an inner or outer surface of a tube.

Embodiment 89. A kit comprising the solid support of any one of embodiments 63 to 88.

Embodiment 90. The kit of embodiment 89, further comprising a transposase.

Embodiment 91. The kit of embodiment 89 or 90 further comprising a reverse transcriptase polymerase.

Embodiment 92. The kit of any one of embodiments 90 to 91, further comprising a second solid support for immobilizing DNA comprising a second transposome complex comprising a transposase and a third polynucleotide comprising a 3′ portion comprising a transposon end sequence, and optionally a second tag.

Embodiment 93. A method of preparing an immobilized library of tagged DNA:RNA fragments from a sample comprising RNA and DNA, comprising (a) applying the sample comprising RNA and DNA to a first solid support for immobilizing DNA comprising first transposome complexes immobilized thereon, wherein the first transposome complexes comprise a transposase and a first polynucleotide comprising a 3′ portion comprising a transposon end sequence, and optionally a first tag; and a second solid support having first capture oligonucleotides immobilized thereon, wherein the sample is applied to the mixture of first and second solid supports under conditions wherein the DNA binds to the first transposome complexes on the first solid support and is fragmented and optionally tagged, and the RNA binds to the first capture oligonucleotides on the second solid support; (b) transferring the RNA bound to the second solid support to a third solid support having second capture oligonucleotides that bind the transferred RNA and second transposome complexes immobilized thereon, wherein the second transposome complexes comprise a transposase and a second polynucleotide comprising a 3′ portion comprising a transposon end sequence, and a second tag; (c) adding a reverse transcriptase polymerase under conditions to synthesize cDNA and generate immobilized DNA:RNA duplexes on the second capture oligonucleotides; and (d) performing tagmentation on the DNA:RNA duplexes with the second transposome complexes under conditions wherein the DNA:RNA duplexes are tagged on the 5′ end of one strand, thereby producing an immobilized library of DNA:RNA fragments wherein at least one strand is 5′-tagged with the first tag.

Embodiment 94. The method of embodiment 93, wherein the first and/or second capture oligonucleotides comprise a polyT sequence.

Embodiment 95. The method of embodiment 93 or embodiment 94, wherein the RNA comprises a sequence complementary to at least a portion of one or more of the first and/or second capture oligonucleotides.

Embodiment 96. The method of any one of embodiments 93 to 95, wherein the first and/or second transposome complexes are immobilized to the solid support via the first and/or second polynucleotides.

Embodiment 97. The method of any one of embodiments 93 to 96, further comprising washing the solid support after step (a) to remove any unbound DNA or RNA.

Embodiment 98. A method of preparing an immobilized library of tagged DNA:RNA fragments from a sample comprising RNA and DNA, comprising (a) applying a sample comprising RNA and DNA to a first solid support for immobilizing DNA comprising first transposome complexes immobilized thereon, wherein the first transposome complexes comprise a transposase and a first polynucleotide comprising a 3′ portion comprising a transposon end sequence, and optionally a first tag; and a second solid support having capture oligonucleotides and a second polynucleotide immobilized thereon, wherein the second polynucleotide comprises a 3′ portion comprising a transposon end sequence, and a second tag; wherein the sample is applied to the mixture of first and second solid supports under conditions wherein the DNA binds to the first transposome complexes on the first solid support and is fragmented and optionally tagged, and the RNA binds to the capture oligonucleotides on the second solid support; (b) adding a transposase under conditions wherein the transposase binds to the second polynucleotide to form transposome complexes on the second solid support; adding a reverse transcriptase polymerase under conditions to synthesize cDNA and generate immobilized DNA:RNA duplexes on the second capture oligonucleotides; and (c) performing tagmentation on the DNA:RNA duplexes with the second transposome complexes under conditions wherein the DNA:RNA duplexes are tagged on the 5′ end of one strand, thereby producing an immobilized library of DNA:RNA fragments wherein at least one strand is 5′-tagged with the first tag.

Embodiment 99. The method of embodiment 98, wherein the capture oligonucleotides comprise a polyT sequence.

Embodiment 100. The method of embodiment 98 or embodiment 99, wherein the RNA comprises a sequence complementary to at least a portion of one or more of the capture oligonucleotides.

Embodiment 101. The method of any one of embodiments 98 to 100, wherein the first and/or second transposome complexes are immobilized to the solid support via the first and/or second polynucleotides.

Embodiment 102. The method of any one of embodiments 98 to 101, further comprising washing the solid support after step (a) to remove any unbound DNA or RNA.

Embodiment 103. A method of preparing an immobilized library of tagged DNA:RNA fragments from a sample comprising RNA and DNA, comprising (a) applying a sample comprising RNA and DNA to a first solid support for immobilizing DNA comprising first transposome complexes immobilized thereon, wherein the first transposome complexes comprise a transposase and a first polynucleotide comprising a 3′ portion comprising a transposon end sequence, and optionally a first tag; and a second solid support for immobilizing RNA having capture oligonucleotides and second transposome complexes that are reversibly deactivated immobilized thereon, wherein the transposome complexes comprise a transposase bound to a second polynucleotide comprising a 3′ portion comprising a transposon end sequence, and a second tag; wherein the sample is applied to the mixture of first and second solid supports under conditions wherein the DNA binds to the first transposome complexes on the first solid support and is fragmented and optionally tagged, and the RNA binds to the capture oligonucleotides on the second solid support; (b) adding a reverse transcriptase polymerase under conditions to synthesize cDNA and generate immobilized DNA:RNA duplexes on the second capture oligonucleotides; (c) activating the second transposome complexes; and (d) performing tagmentation on the DNA:RNA duplexes with the activated second transposome complexes under conditions wherein the DNA:RNA duplexes are tagged on the 5′ end of one strand, thereby producing an immobilized library of DNA:RNA fragments wherein at least one strand is 5′-tagged with the first tag.

Embodiment 104. The method of embodiment 103, wherein the transposome complex is reversibly deactivated by a transposome deactivator bound to the transposome complex.

Embodiment 105. The method of embodiment 104, wherein the transposome deactivator is bound to a Tn5 binding site of the transposome complex.

Embodiment 106. The method of embodiment 104 or embodiment 105, wherein the transposome deactivator comprises dephosphorylated ME′, extra bases, inhibitor duplexes, and/or heat-labile antibodies.

Embodiment 107. The method of any one of embodiments 104 to 106, wherein the transposome complex is activated in step (c) by removal of the transposome deactivator.

Embodiment 108. The method of any one of embodiments 103 to 106, wherein the capture oligonucleotides comprise a polyT sequence.

Embodiment 109. The method of any one of embodiments 103 to 108, wherein the RNA comprises a sequence complementary to at least a portion of one or more of the capture oligonucleotides.

Embodiment 110. The method of any one of embodiments 103 to 109, wherein the first and/or second transposome complexes are immobilized to the solid support via the first and/or second polynucleotides.

Embodiment 111. The method of any one of embodiments 103 to 110, further comprising washing the solid support after step (a) to remove any unbound DNA or RNA.

Embodiment 112. A method of preparing an immobilized library of tagged DNA:RNA fragments from a sample comprising RNA and DNA, comprising (a) applying a sample comprising RNA and DNA to a first solid support for immobilizing DNA comprising first transposome complexes immobilized thereon, wherein the first transposome complexes comprise a transposase and a first polynucleotide comprising a 3′ portion comprising a transposon end sequence, and optionally a first tag, and wherein the sample is applied under conditions wherein the DNA binds to the first transposome complexes on the first solid support and is fragmented and optionally tagged; (b) separating the first solid support with the bound DNA from the RNA; (c) applying the RNA to a second solid support for immobilizing RNA having capture oligonucleotides and second transposome complexes immobilized thereon, wherein the second transposome complexes comprise a transposase bound to a second polynucleotide comprising a 3′ portion comprising a transposon end sequence, and a second tag, and wherein the RNA is applied under conditions wherein the RNA binds to the capture oligonucleotides on the second solid support; (d) adding a reverse transcriptase polymerase under conditions to synthesize cDNA and generate immobilized DNA:RNA duplexes on the second capture oligonucleotides; and (e) performing tagmentation on the DNA:RNA duplexes with the activated second transposome complexes under conditions wherein the DNA:RNA duplexes are tagged on the 5′ end of one strand, thereby producing an immobilized library of DNA:RNA fragments wherein at least one strand is 5′-tagged with the first tag.

Embodiment 113. The method of embodiment 112, wherein the capture oligonucleotides comprise a polyT sequence.

Embodiment 114. The method of embodiment 112 or embodiment 113, wherein the RNA comprises a sequence complementary to at least a portion of one or more of the capture oligonucleotides.

Embodiment 115. The method of any one of embodiments 112 to 114, wherein the first and/or second transposome complexes are immobilized to the solid support via the first and/or second polynucleotides.

Embodiment 116. The method of any one of embodiments 112 to 115, further comprising washing the solid support after step (c) to remove any unbound RNA.

Embodiment 117. The method of any one of embodiments 112 to 116, further comprising recombining the first solid support with the bound DNA with the second solid support with the immobilized library of tagged DNA:RNA fragments.

Embodiment 118. A method of preparing an immobilized library of tagged DNA:RNA fragments from target RNA comprising (a) adding a reverse transcriptase polymerase to a sample comprising target RNA under conditions to synthesize cDNA and generate DNA:RNA duplexes; (b) immobilizing DNA:RNA duplexes to a solid support having transposome complexes immobilized thereon, wherein the transposome complexes comprise a transposase bound to a first polynucleotide comprising a 3′ portion comprising a transposon end sequence and a first tag, wherein the sample is applied to the solid support under conditions wherein the DNA:RNA duplexes bind to capture oligonucleotides or transposases directly; and (b) performing tagmentation on the DNA:RNA duplexes with the transposome complexes under conditions wherein the DNA:RNA duplexes are tagged on the 5′ end of one strand, thereby producing an immobilized library of DNA:RNA fragments wherein at least one strand is 5′-tagged with the first tag.

Embodiment 119. The method of embodiment 118, wherein the transposome complex is immobilized to the solid support via the first polynucleotide.

Embodiment 120. The method of embodiment 118 or embodiment 119, wherein the transposome complexes comprise a second polynucleotide comprising a region complementary to the transposon end sequence.

Embodiment 121. The method of embodiment 120, wherein the transposome complex is immobilized to the solid support via the second polynucleotide.

Embodiment 122. The method of any one of embodiments 118 to 121, wherein the transposome complexes are present on the solid support at a density of at least 10³, 10⁴, 10⁵, or 10⁶ complexes per mm².

Embodiment 123. The method of any one of embodiments 118 to 122, wherein the transposase comprises a Tn5 transposase.

Embodiment 124. The method of embodiment 123, wherein the Tn5 transposase is hyperactive Tn5 transposase.

Embodiment 125. The method of any one of embodiments 118 to 124, wherein the lengths of the double-stranded fragments in the immobilized library are adjusted by increasing or decreasing the density of transposome complexes on the solid support.

Embodiment 126. The method of any one of embodiments 118 to 125, wherein at least 50%, 55%, 60%, 65%, 70%, 75%, 80%, 85%, 90%, 95%, 96%, 97%, 98%, or 99% of the first tags comprise the same tag domain.

Embodiment 127. The method of any one of embodiments 118 to 126, wherein the tag comprises a region for cluster amplification.

Embodiment 128. The method of any one of embodiments 118 to 127, wherein the tag comprises a region for priming a sequencing reaction.

Embodiment 129. The method of any one of embodiments 118 to 128, wherein the solid support comprises microparticles, beads, a planar support, a patterned surface, or wells.

Embodiment 130. The method of embodiment 129, wherein the planar support is an inner or outer surface of a tube.

Embodiment 131. The method of any one of embodiments 118 to 130, wherein performing tagmentation produces double stranded DNA:RNA duplexes bridged to two immobilized transposome complexes on the solid support.

Embodiment 132. The method of embodiment 131, wherein the length of the bridged duplexes is from 100 base pairs to 1500 base pairs.

Embodiment 133. The method of any one of the embodiments 118 to 132, wherein the sample that is applied to the solid support is blood.

Embodiment 134. The method of any one of embodiments 118 to 133, wherein the sample that is applied to the solid support is a cell lysate.

Embodiment 135. The method of embodiment 134, wherein the cell lysate is a crude cell lysate.

Embodiment 136. The method of any one of embodiments 118 to 135, wherein the sample that is applied to the solid support has a 260/280 absorbance ratio that is less than or equal to 1.7.

Embodiment 137. The method of any one of embodiments 118 to 136, further comprising lysing cells in the sample after applying the sample to the solid support.

Embodiment 138. The method of any one of embodiments 118 to 137, further comprising: (d) contacting solution-phase transposome complexes with the immobilized DNA:RNA fragments under conditions whereby the DNA:RNA fragments are further fragmented by the solution-phase transposome complexes; thereby obtaining immobilized nucleic acid fragments having one end in solution.

Embodiment 139. The method of embodiment 138, wherein the solution-phase transposome complexes comprise a second tag, thereby generating immobilized nucleic acid fragments having a second tag in solution.

Embodiment 140. The method of embodiment 139, wherein the first and second tags are different.

Embodiment 141. The method of any one of embodiments 138 to 140, wherein at least 50%, 55%, 60%, 65%, 70%, 75%, 80%, 85%, 90%, 95%, 96%, 97%, 98%, or 99% of the solution-phase transposome complexes comprise a second tag.

Embodiment 142. The method of any one of embodiments 138 to 141, further comprising amplifying the fragments on the solid support by reacting a polymerase and an amplification primer corresponding to a portion of the first polynucleotide.

Embodiment 143. The method of any one of embodiments 1-142, wherein the 5′ end of one strand is the 5′ end of the RNA strand.

Embodiment 144. The method of any one of embodiments 1-142, wherein the 5′ end of one strand is the 5′ end of the DNA strand.

Embodiment 145. A method of preparing an immobilized library of tagged fragments from a sample comprising RNA and DNA, wherein the tagged fragments comprise either a DNA-specific barcode or an RNA-specific barcode, comprising (a) combining a sample comprising RNA and DNA with a first solid support for immobilizing DNA, wherein the first solid support comprises transposome complexes immobilized thereon, wherein the transposome complexes comprise a transposase and a transposon comprising a transposon end sequence and a DNA-specific barcode; (b) immobilizing the DNA; (c) performing tagmentation on the first solid support to prepare tagged fragments comprising a DNA-specific barcode; (d) preparing double-stranded cDNA from the RNA; (e) combining the sample with a second solid support for immobilizing cDNA, wherein the second solid support comprises transposome complexes immobilized thereon, wherein the transposome complexes comprise a transposase and a transposon comprising a transposon end sequence and an RNA-specific barcode; and (f) immobilizing the cDNA and performing tagmentation on the second solid support to prepare tagged fragments comprising an RNA-specific barcode, optionally wherein transposome complexes are reversibly deactivated before performing tagmentation and performing tagmentation comprises activating the transposome complexes.

Embodiment 146. The method of embodiment 145, further comprising combining the first and second solid supports after performing tagmentation on the second solid support, wherein each solid support has immobilized tagged fragments comprising either a DNA-specific barcode or an RNA-specific barcode.

Embodiment 147. The method of embodiment 145 or embodiment 146, further comprising partitioning the first solid support with the immobilized tagged fragments comprising a DNA-specific barcode from the rest of the sample after performing tagmentation on the first solid support and before preparing double-stranded cDNA from the RNA.

Embodiment 148. The method of embodiment 145 or embodiment 146, further comprising partitioning the first solid support with the immobilized DNA from the rest of the sample after immobilizing the DNA and before performing tagmentation on the first solid support to prepare tagged fragments comprising a DNA-specific barcode.

Embodiment 149. The method of any one of embodiments 145-148, wherein the preparing double-stranded cDNA from the RNA is performed by template switching.

Embodiment 150. A method of preparing an immobilized library of tagged fragments from a sample comprising RNA and DNA, wherein the tagged fragments comprise either a DNA-specific barcode or an RNA-specific barcode, comprising (a) combining a sample comprising RNA and DNA with a first solid support for immobilizing DNA, wherein the first solid support comprises transposome complexes immobilized thereon, wherein the transposome complexes comprise a transposase and a transposon comprising a transposon end sequence and a DNA-specific barcode; (b) immobilizing the DNA; (c) performing tagmentation on the first solid support to prepare tagged fragments comprising a DNA-specific barcode; (d) preparing a single strand of cDNA from the RNA to produce DNA:RNA duplexes; (e) combining the sample with a second solid support for immobilizing DNA:RNA duplexes, wherein the second solid support comprises transposome complexes immobilized thereon, wherein the transposome complexes comprise a transposase with activity on DNA:RNA duplexes and a transposon comprising a transposon end sequence and an RNA-specific barcode; and (f) immobilizing the DNA:RNA duplexes and performing tagmentation on the second solid support to prepare tagged fragments comprising an RNA-specific barcode, optionally wherein transposome complexes are reversibly deactivated before performing tagmentation and performing tagmentation comprises activating the transposome complexes.

Embodiment 151. The method of embodiment 150, further comprising combining the first and second solid supports after performing tagmentation on the second solid support, wherein each solid support has immobilized tagged fragments comprising either a DNA-specific barcode or an RNA-specific barcode.

Embodiment 152. The method of embodiment 150 or embodiment 151, further comprising partitioning the first solid support with the immobilized tagged fragments comprising a DNA-specific barcode from the rest of the sample after performing tagmentation on the first solid support and before preparing a single strand of cDNA from the RNA to produce DNA:RNA duplexes.

Embodiment 153. The method of embodiment 150 or embodiment 151, further comprising partitioning the first solid support with the immobilized DNA from the rest of the sample after immobilizing the DNA and before performing tagmentation on the first solid support to prepare tagged fragments comprising a DNA-specific barcode.

Embodiment 154. A method of preparing an immobilized library of tagged fragments from a sample comprising RNA and DNA, wherein the tagged fragments comprise either a DNA-specific barcode or an RNA-specific barcode, comprising (a) combining a sample comprising RNA and DNA with a first solid support for immobilizing DNA, wherein the first solid support comprises transposome complexes immobilized thereon, wherein the transposome complexes comprise a transposase and a transposon comprising a transposon end sequence and a DNA-specific barcode; (b) immobilizing the DNA; (c) performing tagmentation on the first solid support to prepare tagged fragments comprising a DNA-specific barcode; (d) preparing double-stranded cDNA from the RNA; (e) performing tagmentation on the double-stranded DNA in solution, wherein the transposome complexes in solution comprise a transposase and a transposon comprising a transposon end sequence, an RNA-specific barcode, and a sequence that hybridizes to capture probes, to prepare tagged fragments of the double-stranded cDNA, optionally wherein transposome complexes are reversibly deactivated before performing tagmentation and performing tagmentation comprises activating the transposome complexes, and wherein the tagged fragments comprise the RNA-specific barcode and the sequence that hybridizes to capture probes; (f) combining the sample with a second solid support having a surface comprising capture probes; and (g) immobilizing the tagged fragments of double-stranded cDNA on the second solid support.

Embodiment 155. The method of embodiment 154, further comprising combining the first and second solid supports after immobilizing the tagged fragments of double-stranded cDNA on the second solid support, wherein each solid support has immobilized tagged fragments comprising either a DNA-specific barcode or an RNA-specific barcode.

Embodiment 156. The method of embodiment 154 or embodiment 155, further comprising partitioning the first solid support with the immobilized tagged fragments comprising a DNA-specific barcode from the rest of the sample after performing tagmentation on the first solid support and before double-stranded cDNA from the RNA.

Embodiment 157. The method of embodiment 154 or embodiment 155, further comprising partitioning the first solid support with the immobilized DNA from the rest of the sample after immobilizing the DNA and before performing tagmentation on the first solid support to prepare tagged fragments comprising a DNA-specific barcode.

Embodiment 158. A method of preparing an immobilized library of tagged fragments from a sample comprising RNA and DNA, wherein the tagged fragments comprise either a DNA-specific barcode or an RNA-specific barcode, comprising (a) combining a sample comprising RNA and DNA with a first solid support for immobilizing DNA, wherein the first solid support comprises transposome complexes immobilized thereon, wherein the transposome complexes comprise a transposase and a transposon comprising a transposon end sequence and a DNA-specific barcode; (b) immobilizing the DNA; (c) performing tagmentation on the first solid support to prepare tagged fragments comprising a DNA-specific barcode; (d) preparing a single strand of cDNA from the RNA to produce DNA:RNA duplexes; (e) performing tagmentation on the DNA:RNA duplexes in solution, wherein the transposome complexes in solution comprise a transposase and a transposon comprising a transposon end sequence, an RNA-specific barcode, and a sequence that hybridizes to capture probes, to prepare tagged fragments of the DNA:RNA duplexes, optionally wherein transposome complexes are reversibly deactivated before performing tagmentation and performing tagmentation comprises activating the transposome complexes, and wherein the tagged fragments comprise the RNA-specific barcode and the sequence that hybridizes to capture probes; (f) combining the sample with a second solid support having a surface comprising capture probes; and (g) immobilizing the tagged fragments of DNA:RNA duplexes on the second solid support.

Embodiment 159. The method of embodiment 158, further comprising combining the first and second solid supports after immobilizing the tagged fragments of DNA:RNA duplexes on the second solid support, wherein each solid support has immobilized tagged fragments comprising either a DNA-specific barcode or an RNA-specific barcode.

Embodiment 160. The method of embodiment 158 or embodiment 159, further comprising partitioning the first solid support with the immobilized tagged fragments comprising a DNA-specific barcode from the rest of the sample after performing tagmentation on the first solid support and before preparing a single strand of cDNA from the RNA to produce DNA:RNA duplexes.

Embodiment 161. The method of embodiment 158 or embodiment 160, further comprising partitioning the first solid support with the immobilized DNA from the rest of the sample after immobilizing the DNA and before performing tagmentation on the first solid support to prepare tagged fragments comprising a DNA-specific barcode.

Embodiment 162. The method of any one of embodiments 154-161, wherein the capture probes comprise nucleic acids.

Embodiment 163. The method of any one of embodiments 145-162, further comprising adding a synthetic double-stranded DNA to the first solid support after performing tagmentation on the first solid support.

Embodiment 164. The method of embodiment 163, wherein the synthetic double-stranded DNA comprises uracil.

Embodiment 165. The method of any one of embodiments 145-164, wherein the DNA-specific barcode and the RNA-specific barcode comprise different primer binding sequences.

Embodiment 166. The method of embodiment 165, further comprising amplifying tagged fragments comprising the DNA-specific barcode using a primer that binds the primer binding sequence comprised in the DNA-specific barcode.

Embodiment 167. The method of embodiment 165, further comprising amplifying tagged fragments comprising the RNA-specific barcode using a primer that binds the primer binding sequence comprised in the RNA-specific barcode.

Embodiment 168. The method of embodiment 165, further comprising amplifying tagged fragments comprising the DNA-specific barcode and tagged fragments comprising the RNA-specific barcode using a primer mix comprising a primer that binds the primer binding sequence comprised in the DNA-specific barcode and a primer that binds the primer binding sequence comprised in the RNA-specific barcode.

Embodiment 169. The method of any one of embodiments 166 to 168, wherein the amplifying is performed with a uracil-intolerant DNA polymerase.

Embodiment 170. The method of any one of embodiments 168-169, wherein the amplifying comprises bridge amplification.

Embodiment 171. The method of any one of embodiments 145-170, further comprising sequencing the tagged fragments or amplified tagged fragments.

Embodiment 172. The method of any one of embodiments 145-171, wherein the method is performed in a single reaction vessel.

Embodiment 173. The method of any one of embodiments 145-172, wherein the transposome complexes are present on the solid support at a density of at least 10³, 10⁴, 10⁵, or 10⁶ complexes per mm².

Embodiment 174. The method of any one of embodiments 145-173, wherein the transposase comprises a Tn5 transposase.

Embodiment 175. The method of embodiment 174, wherein the Tn5 transposase is hyperactive Tn5 transposase.

Embodiment 176. The method of any one of embodiments 145-175, wherein the lengths of the immobilized fragments are adjusted by increasing or decreasing the density of transposome complexes on the solid support.

Embodiment 177. The method of any one of embodiments 145-176, wherein the solid supports comprise microparticles, beads, a planar support, a patterned surface, or wells.

Embodiment 178. The method of embodiment 177, wherein the solid supports are beads.

Embodiment 179. Solid supports having a library of tagged fragments immobilized thereon prepared according to the method of any one of embodiments 145-178.

Embodiment 180. The solid supports of embodiment 179, wherein the solid supports are beads.

Embodiment 181. A method of preparing strand-specific libraries of single-stranded DNA from RNA comprising (a) preparing a first strand of cDNA from an RNA comprised in a sample using a reverse transcriptase, a primer, and nucleotides comprising dTTP under conditions that inhibit DNA-dependent DNA synthesis; (b) preparing a second strand of cDNA from the first strand of cDNA using a DNA polymerase, a primer, and nucleotides comprising dUTP to prepare double-stranded cDNA; (c) applying the double-stranded cDNA to a solid support having transposome complexes immobilized thereon, wherein each transposome complex comprises a transposase; a first transposon comprising a 3′ portion comprising a transposon end sequence and a first-read sequencing adapter sequence; wherein the first transposon comprises a 5′ affinity element for immobilizing the transposome complex to the solid support; and a second transposon sequence comprising a sequence all or partially complementary to the transposon end sequence; (b) performing tagmentation on the double-stranded DNA with the transposome complexes to prepare tagged double-stranded DNA fragments comprising the first-read sequencing adapter sequence, optionally wherein transposome complexes are reversibly deactivated before performing tagmentation and performing tagmentation comprises activating the transposome complexes; (c) removing the second transposon and performing gap-filling and extension; (d) separating the strands of the double-stranded DNA fragments; (e) hybridizing a primer comprising a second-read sequencing adapter sequence to the transposon end sequence or the sequence all or partially complementary to the transposon end sequence and amplifying to prepare a DNA strand that is not attached to the solid support and that comprises the first-read sequencing adapter and the second- read sequencing adapter; and (f) releasing the strand generating by the amplifying from the solid support, wherein the releasing releases a single-stranded DNA fragment comprising the first-read sequencing adapter and the second-read sequencing adapter.

Embodiment 182. The method of embodiment 181, wherein the conditions that inhibit DNA-dependent DNA synthesis is the presence of a buffer comprising actinomycin D.

Embodiment 183. The method of any one of embodiments 181 to 182, wherein the primer is one or more randomer primers.

Embodiment 184. The method of any one of embodiments 181-183, wherein the primer is a mix of a randomer primer and a polyT primer.

Embodiment 185. The method of any one of embodiments 181-184, wherein the primer for the preparing a second strand of cDNA is the same as the primer for the preparing a first strand of cDNA.

Embodiment 186. The method of any one of embodiments 181-185, wherein the RNA is a long non-coding RNA or antisense transcript.

Embodiment 187. The method of any one of embodiments 181-186, wherein the amplifying is performed with a uracil-intolerant polymerase.

Embodiment 188. The method of embodiment 187, wherein the amplifying does not amplify from a DNA strand comprising uracil.

Embodiment 189. The method of any one of embodiments 181-188, wherein a unique molecular identifier (UMI) is comprised in the primer comprising a second-read sequencing adapter sequence.

Embodiment 190. The method of embodiment 189, wherein the UMI is located between the second-read sequencing adapter sequence and the sequence that can bind to the transposon end sequence or the sequence all or partially complementary to the transposon end sequence.

Embodiment 191. The method of any one of embodiments 181-188, wherein a UMI is comprised in the first transposon.

Embodiment 192. The method of embodiment 191, wherein the UMI is located between the transposon end sequence and the first-read sequencing adapter sequence.

Embodiment 193. The method of any one of embodiments 189-192, wherein the RNA comprises a pool of different RNAs and the single-stranded fragment comprising the first-read sequencing adapter and the second-read sequencing adapter comprises a pool of different fragments, wherein each fragment comprises a UMI that is different from other fragments comprised in the pool of different fragments.

Embodiment 194. The method of any one of embodiments 181-193, wherein the affinity element is a biotin or desthiobiotin and the solid support comprises streptavidin or avidin on its surface.

Embodiment 195. The method of embodiment 194, wherein the affinity element is a dual biotin.

Embodiment 196. The method of any one of embodiments 181-195, wherein the releasing is performed with heat or sodium hydroxide treatment.

Embodiment 197. The method of any one of embodiments 181-196, wherein the single-stranded fragment comprising the first-read sequencing adapter and the second-read sequencing adapter is partitioned from the solid support after the releasing.

Embodiment 198. The method of any one of embodiments 181-197, further comprising performing index primer amplification with the single-stranded DNA fragment comprising the first-read sequencing adapter and the second-read sequencing adapter to prepare an indexed fragment after the releasing.

Embodiment 199. The method of embodiment 198, wherein the index primer amplification is performed in a separate reaction vessel from the solid support.

Embodiment 200. The method of embodiment 198 or embodiment 199, wherein the index primer amplification is performed with a uracil-intolerant polymerase.

Embodiment 201. The method of any one of embodiments 181-200, further comprising sequencing the single-stranded DNA fragment comprising the first-read sequencing adapter and the second-read sequencing adapter or the indexed fragment.

Embodiment 202. The method of embodiment 201, wherein sequencing data is generated from the first strand of cDNA generated from the RNA.

Embodiment 203. The method of embodiment 201, wherein sequencing data is not generated from the second strand of cDNA generated from the RNA.

Embodiment 204. The method of any one of embodiments 181-203, wherein the method does not require ligation.

Embodiment 205. The method of any one of embodiments 181-206, wherein the method demarcates the boundaries of overlapping sequences in the RNA.

Embodiment 206. The method of any one of embodiments 181-207, wherein the method allows estimate of transcript expression.

Embodiment 207. The method of embodiment 208, wherein the estimate of transcript expression is based on analysis of UMIs.

Embodiment 208. A method of preparing a library of double-stranded DNA fragments from RNA comprising (a) preparing a first strand of cDNA from a full-length RNA in a sample using a polyT primer comprising a UMI and a first-read sequencing adapter sequence; (b) preparing a second strand of cDNA to generate double-stranded cDNA; (c) applying the double- stranded cDNA to a bead having transposome complexes immobilized thereon, wherein each transposome complex comprises a transposase; a first transposon comprising a 3′ transposon end sequence; and a second transposon comprising a sequence all or partially complementary to the transposon end sequence and a hybridization sequence, wherein the transposome complex is immobilized by binding of the hybridization sequence to an oligonucleotide immobilized to a bead, wherein said oligonucleotide comprises a 5′ affinity element, a first-read sequencing adapter sequence, a bead code, and a sequence all or partially complementary to the hybridization sequence; (b) immobilizing the double-stranded cDNA and performing tagmentation on the bead to prepare double-stranded DNA fragments, optionally wherein transposome complexes are reversibly deactivated before performing tagmentation and performing tagmentation comprises activating the transposome complexes; (c) removing the second transposon; (d) hybridizing a primer comprising a second-read sequencing adapter sequence and a sequence all or partially complementary to the transposon end sequence to the transposon end sequence; and (e) performing gap-filling and extension to prepare double-stranded DNA fragments comprising the first-read sequencing adapter and the second-read sequencing adapter.

Embodiment 209. A method of preparing a library of double-stranded DNA fragments from RNA comprising (a) preparing a first strand of cDNA from a full-length RNA in a sample using a polyT primer comprising a UMI and a first-read sequencing adapter sequence; (b) preparing a second strand of cDNA to generate double-stranded cDNA; (c) applying the double-stranded cDNA to a bead having transposome complexes immobilized thereon, wherein each transposome complex comprises a transposase; a first transposon comprising a 3′ transposon end sequence, a bead code, and a second-read sequencing adapter sequence; wherein the first transposon further comprises a 5′ affinity element for immobilizing the transposome complex to the solid support; and a second transposon comprising a sequence all or partially complementary to the transposon end sequence; (b) immobilizing the double-stranded cDNA and performing tagmentation on the bead to prepare double-stranded DNA fragments, optionally wherein transposome complexes are reversibly deactivated before performing tagmentation and performing tagmentation comprises activating the transposome complexes; (c) removing the second transposon; (d) hybridizing a primer comprising a second-read sequencing adapter sequence and a sequence all or partially complementary to the transposon end sequence to the transposon end sequence; and (e) performing gap-filling and extension to prepare double-stranded DNA fragments comprising the first-read sequencing adapter and the second-read sequencing adapter.

Embodiment 210. The method of embodiment 208 or 209, wherein the sequence all or partially complementary to the transposon end sequence is shorter than the transposon end sequence.

Embodiment 211. The method of embodiment 210, wherein fewer adapter dimers are generated when the sequence all or partially complementary to the transposon end sequence is shorter than the transposon end sequence

Embodiment 212. The method of any one of embodiments 208-211, wherein the primer comprises a 5′ portion comprising the second-read sequence adapter and a 3′ portion comprising the sequence all or partially complementary to the transposon end sequence.

Embodiment 213. The method of any one of embodiments 210-212, wherein the fragments remain attached to a transposome at one or both end when removing the sequence all or partially complementary to the transposon end sequence.

Embodiment 214. The method of any one of embodiments 210-213, wherein the full-length RNA comprises a pool of different full-length RNAs and the polyT primer comprises a pool of different polyT primers comprising different UMIs.

Embodiment 215. The method of embodiment 214, wherein each polyT primer comprised in the pool of different polyT primers comprises a different UMI.

Embodiment 216. The method of embodiment 210-215, wherein the full-length RNA comprises a pool of different full-length RNAs and the 3′ double-stranded DNA fragment prepared from a single full-length RNA comprises a UMI that is different from the 3′ double-stranded DNA fragments prepared from other full-length RNAs in the pool.

Embodiment 217. The method of embodiment 216, wherein the full-length RNA comprises a pool of different full-length RNAs and the bead comprises a pool of beads.

Embodiment 218. The method of embodiment 217, wherein each bead has immobilized a transposome complexes comprising a different bead code as compared to the bead code comprised in transposome complexes immobilized on other beads in the pool.

Embodiment 219. The method of any one of embodiments 210-218, wherein all the fragments prepared from a double-stranded cDNA prepared from a single full-length RNA are tagmented on the same bead.

Embodiment 220. The method of any one of embodiments 210-219, wherein all the double-stranded fragments comprising the first-read sequencing adapter and the second-read sequencing adapter prepared from a double-stranded cDNA are on the same solid support after performing gap-filling and extension.

Embodiment 221. The method of any one of embodiments 210-220, wherein the full-length RNA comprises a pool of different full-length RNAs and all the double-stranded fragments comprising the first-read sequencing adapter and the second-read sequencing adapter prepared from a single full-length RNA in the pool are on the same solid support after performing gap-filling and extension.

Embodiment 222. The method of any one of embodiments 210-221, further comprising amplifying the double-stranded fragments comprising the first-read sequencing adapter and the second-read sequencing adapter to prepare amplified fragments.

Embodiment 223. The method of any one of embodiments 210-222, further comprising sequencing the amplified fragments or the double-stranded fragments comprising the first-read sequencing adapter and the second-read sequencing adapter.

Embodiment 224. The method of embodiment 223, wherein the sequencing allows full-length RNA isoform detection.

Embodiment 225. The method of any one of embodiments 210-224, wherein the double-stranded cDNA preparation is by a stranded method.

Embodiment 226. The method of any one of embodiments 223-225, wherein the presence of a bead code in a sequence obtained from a double-stranded fragment comprising the first-read sequencing adapter and the second-read sequencing adapter or amplified fragments identifies the bead on which the fragment was generated.

Embodiment 227. The method of any one of embodiments 210-226, wherein the sample is a single cell.

Embodiment 228. The method of any one of embodiments 210-227, wherein the preparing double-stranded cDNA from the RNA and the combining the sample with a second solid support for immobilizing cDNA comprise the method of any one of embodiments 145.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-1C compare the current method of fragmentation of RNA:DNA duplexes by immobilized transposomes (A) versus fragmentation of DNA:RNA duplexes by transposomes in solution by capture and tagmentation (Cap-Tag, B and C). In both FIG. 1A and FIG. 1B, target RNA is immobilized by polyT capture oligonucleotides that bind to the polyA tails of the mRNA. In FIG. 1B and 1C, it is proposed that the reverse transcriptase initiates second stand synthesis via nicks that occur in the RNA strand and that these dsDNA duplexes that are the substrates for the transposomes. As a consequence of using transposomes from solution, however, reads are only generated from the 3′end of the transcripts. In contrast, the present method with immobilized transposomes (FIG. 1A) allow library generation corresponding to the full length of the target RNA.

FIG. 2 shows how DNA:RNA duplexes in solution can been tagmented by transposomes immobilized on a surface. The target RNA is used for cDNA synthesis to generate the DNA:RNA duplex followed by tagmentation of the DNA:RNA duplex via immobilized transposome complexes. The tagmentation produces double-stranded DNA:RNA duplexes bridged to two immobilized transposome complexes on the solid support. The activity of the transposase of the transposome complexes (such as Tn5 as shown here) is then stopped or the transposase is removed. Strand exchange is performed followed by gap-fill ligation. The library of fragments can be released either in a tube or flowcell.

FIGS. 3A-3C shows representative sequencing results from an RNA sequencing library generated using cDNA synthesis from universal human reference RNA to generate DNA:RNA duplexes. These DNA:RNA duplexes were tagmented by immobilized transposome complexes (as shown in FIG. 2), such as BLTs. After tagmentation by the immobilized transposomes complexes on BLTs, gap-fill ligation was performed. The library was released into a tube and seeded directly on a flowcell. The library was sequencable (A). The distribution of results showed sequencing of coding, untranslated region (UTR), introns, and intergenic sequences (B), and thus indicates that the different regions of the RNA could be sequenced using the present method. The normalized coverage along the transcript length is also shown (C), which supports a relative lack of 3′ bias in the sequencing results.

FIG. 4 shows co-encapsulation of a cell and a capture bead in a droplet, followed by cell lysis, cDNA synthesis, and library preparation. A bead with an immobilized library of fragments can be delivered to a solid surface for sequencing (such as a flowcell) before the library fragments are released. Alternatively, a bead with an immobilized target nucleic acid can be delivered to a solid surface for sequencing, followed by preparing of fragments on the bead and releasing of fragments onto a solid surface for sequencing. The release of fragments onto the flowcell can allow for on-flowcell spatial reads to identify fragments that likely originated from the same cell.

FIG. 5 shows beads comprising a capture oligonucleotide. In this representative example, the capture oligonucleotide comprises a polyT sequence, a barcode (that can be used as a bead code), and a P5 adapter sequence. The polyT sequence can be used to capture mRNA, and the P5 and barcode can be incorporated during preparation of a DNA:RNA duplex. Other adapters (show here as B′-P7′) can be incorporated during or after tagmentation. After tagmentation, strand exchange and ligation can be performed to prepare library fragments for sequencing.

FIG. 6 shows a bead with a capture oligonucleotide (here a polyT sequence for capturing mRNA) and an immobilized oligonucleotide comprising a sequence (“shortA”) for hybridizing to a hybridization sequence comprised in a second transposon comprised in a transposome complex. In this way, the immobilized oligonucleotide can be used for assembling a transposome complex. The immobilized oligonucleotide may further comprise a P5 adapter sequence and a bead code.

FIG. 7 shows a workflow to produce a library from a full-length RNA using activatable BLTs with immobilized oligonucleotides for assembling transposomes, including steps of mRNA capture in a droplet, cDNA synthesis in bulk, hybridization (Hyb) of transposomes to the immobilized oligonucleotides, and tagmentation. The BLT may be captured on a flowcell before or after the tagmentation.

FIG. 8 shows methods for single cell resolution of a library using in a droplet or capture in a microwell of a flowcell.

FIG. 9 shows a method of cDNA synthesis, followed by tagmentation and library preparation on a BLT, and then release of fragments in a flowcell or tube. This method uses a polyT primer to bind to poly-A tails of mRNA to prepare a full-length mRNA library.

FIG. 10 shows a method of cDNA synthesis, followed by tagmentation and library preparation on a BLT, and then release of fragments in a flowcell or tube. This method uses random primers to bind to all RNA to prepare a total RNA library.

FIGS. 11A-11C show a variety of different beads that may be used for the present methods for preparing libraries from RNA. (A) Bead for full-length mRNA library preparation with a capture oligonucleotide (here a polyT sequence for capturing mRNA) and an immobilized oligonucleotide comprising a sequence for hybridizing to a hybridization sequence comprised in a second transposon comprised in a transposome complex (e.g., “shortA”), which can be used for assembling a transposome complex. (B) Hybridization of transposomes to the shortA sequence. (C) Preparation of multiple fragments of the full-length mRNA on the bead.

FIG. 12 shows a representative bead for full-length mRNA library preparation. A bead would have multiple capture oligonucleotides (here polyT sequences for capturing mRNA) and multiple immobilized oligonucleotides comprising a sequence for hybridizing to a hybridization sequence comprised in a second transposon. Before or after preparation of DNA:RNA duplexes, the immobilized oligonucleotide could be used to hybridize and immobilize transposome complexes on the bead.

FIG. 13 shows a representative example of how multiple transposomes on a BLT prepare fragments from the full-length of a DNA:RNA duplex generated from an mRNA.

FIG. 14 shows symmetrical tagmentation events on BLTs after incorporation of a 3′ UMI during synthesis of cDNA.

FIG. 15 shows tagmentation after preparation of a double-stranded cDNA with a 3′ UMI. In this example, each bead comprises an immobilized first transposon with a bead code, allowing a UMI counting assay across an entire mRNA transcript.

FIG. 16 shows tagmentation with a bead after preparation of a double-stranded cDNA with a 3′ UMI, wherein the method comprises first strand cDNA synthesis, template switching, and PCR amplification before tagmentation.

FIG. 17 shows a primer extension strand-specific BLT (PRESS-BLT) workflow with strand-specific cDNA synthesis.

FIG. 18 shows a summary of a PRESS-BLT workflow, wherein the first transposon is immobilized to a bead using a dual biotin.

FIG. 19 summarizes how dual biotin and shortened ME′ sequences may improve yield of workflows. For example, shortened ME′ sequences may be useful in methods such as those shown in FIG. 14 or other methods where a non-transferred ME′ strand is exchanged with a different oligonucleotide.

FIG. 20 shows a diagram of an exemplary workflow for preparing an RNA sequencing library. RNA BLTs comprising polyT capture oligonucleotides are used to capture mRNA from a total RNA sample, the sample is washed, a reverse transcriptase is used to generate cDNA to form a DNA:RNA duplex, and the DNA:RNA duplex is fragmented via the transposome complexes immobilized on the RNA BLT. In embodiments shown in FIGS. 20-28, double- stranded cDNA may also be prepared (instead of a DNA:RNA duplex) and be fragmented via the transposome complexes immobilized on the RNA BLT.

FIG. 21 shows summary of how DNA and RNA in a mixed sample (i.e., comprising both DNA and RNA) might bind to RNA BLTs and DNA BLTs. As RNA does not have affinity for transposome complexes, RNA would only bind to RNA BLTs comprising polyT capture oligonucleotides. However, DNA could bind to either DNA BLTs or RNA BLTs based on the affinity of DNA for the transposon end sequences comprised in the immobilized transposome complexes on both DNA and RNA BLTs. Therefore, methods for use with samples comprising RNA and DNA must selectively segregate DNA to the DNA BLTs. Methods for use with mixes samples comprising DNA and RNA can employ different tags during the tagmentation reaction, such that an iDNA is an index identifying DNA BLTs and iRNA is an index for identifying RNA BLTs. In this way, fragments originating from DNA can be differentiated from fragments originating from RNA.

FIG. 22 outlines a method of preparing RNA and DNA sequencing samples from a sample comprising RNA and DNA by reversibly deactivated RNA BLTs prior to applying the sample. In this way, DNA in the sample binds to the DNA BLTs and only RNA binds to the RNA BLTs (via the polyT capture oligonucleotides). The DNA bound to the DNA BLTs is tagmented, and the beads are washed. The transposome complexes of the RNA BLTs are then activated (i.e., the deactivation is reversed). Reverse transcription is performed to generate DNA:RNA duplexes immobilized to the RNA BLTs, followed by fragmentation of the DNA:RNA duplexes.

FIG. 23 outlines a method of preparing RNA and DNA sequencing samples from a sample comprising RNA and DNA using “naked” RNA BLTs. These “naked” RNA-BLTs do not comprise transposase. First, DNA is tagmented with DNA-BLTs and RNA is captured on the polyT capture oligonucleotides and cDNA synthesis can be performed, the samples are washed, and then transposase are added to assemble functional transposomes on RNA-BLTs that were previously “naked.” Reverse transcriptase polymerase is added and tagmentation of DNA:RNA duplexes is performed with the RNA BLTs. Transposomes can be assembled on a bead after cDNA synthesis by hybridization of transposome complexes to oligonucleotides immobilized to RNA-BLTs.

FIG. 24 outlines a method of preparing RNA and DNA sequencing samples from a sample comprising RNA and DNA using a 3-bead approach. In the 3-bead approach, the method starts with DNA-BLT and polyT bead (lacking transposomes). The DNA is tagmented by the DNA BLTs and the RNA is captured by the polyT beads. The beads are washed. Then RNA- BLTs are added together with a reagent that displaces the captured RNA from polyT bead and onto the RNA-BLTs. Reverse transcriptase polymerase is added and tagmentation of DNA:RNA duplexes is performed with the RNA BLTs.

FIG. 25 outlines a method of preparing RNA and DNA sequencing samples from a sample comprising RNA and DNA using DNA capture followed by physical segregation of DNA-bound DNA BLTs from RNA BLTs. Excess DNA BLTs can be used to bind all the double-stranded (ds) DNA in the samples and perform tagmentation on the DNA. The RNA can then be partitioned from the tagmented DNA BLTs and bound to RNA BLTs. After generating DNA:RNA duplexes via a reverse transcriptase polymerase, tagmentation of the RNA is performed. The RNA BLTs and DNA BLTs can then be combined.

FIG. 26 describes a method of preparing libraries of tagged fragments prepared from DNA or RNA (converted to cDNA) using DNA-specific tagmentation and cDNA-specific tagmentation and partitioning. After combining a sample with total nucleic acid (TNA, comprising DNA and RNA) with DNA BLTs and performing DNA-specific tagmentation to incorporate a DNA-specific barcode into fragments, the DNA BLTs can be partitioned while the DNA fragments are associated with the BLTs. Then, double-stranded cDNA (ds-cDNA) can be generated from RNA and tagmented via RNA (cDNA)-specific tagmentation to incorporate an RNA-specific barcode into fragments. In this way, fragments are tagged such that downstream sequencing and bioinformatics can be used to differentiate samples originating from RNA from those originating from DNA using the barcodes.

FIG. 27 describes a method of preparing libraries of tagged fragments prepared from DNA or RNA (converted to cDNA) using DNA-specific tagmentation and RNA (cDNA)-specific tagmentation. This method may be performed in a single reaction vessel (i.e., a one pot reaction). After DNA-specific tagmentation is performed to incorporate a DNA-specific barcode using DNA BLTs, “suicide” synthetic DNA can be added to these DNA BLTs. Such synthetic DNA may be double-stranded DNA (dsDNA) comprising uracil, which cannot be amplified when a uracil-intolerant DNA polymerase is used for amplification. In this way, all the DNA BLTs will be saturated either with dsDNA from the sample or synthetic dsDNA, and the DNA BLTs (comprising transposome complexes with DNA-specific barcodes) will not be available for further tagmentation. Then, ds-cDNA can be prepared from the RNA in the sample, and cDNA-specific tagmentation can be used to incorporate an RNA-barcode using RNA BLTs comprising transposome complexes with RNA-specific barcodes. After clean-up and amplification, tagged fragments can be sequenced, and the barcodes are used to determine those samples that originate from DNA versus those samples that originate from RNA.

FIG. 28 describes a method for preparation of libraries of tagged fragments prepared from DNA or RNA (converted to DNA:RNA duplexes) using DNA-specific tagmentation and DNA:RNA duplex-specific tagmentation. After DNA-specific tagmentation is performed to incorporate a DNA-barcode using DNA BLTs comprising transposome complexes with DNA-specific barcodes, “suicide” synthetic DNA can be added to these DNA BLTs. Then, a single strand of cDNA can be prepared from the RNA in the sample to generate DNA:RNA duplexes, and tagmentation of the DNA:RNA duplexes can be used to incorporate an RNA-barcode using RNA BLTs comprising transposome complexes with RNA-specific barcodes. Exemplary RNA BLTs include tedRNA beads from xGEN USA with improved efficacy at tagmenting DNA:RNA duplexes. After clean-up and amplification, tagged fragments can be sequenced, and the barcodes are used to determine those samples that originate from DNA versus those samples that originate from RNA.

### DESCRIPTION OF THE EMBODIMENTS

### 1. Methods of Preparing RNA Sequencing Libraries Using Bead-Linked Transposomes Comprising Capture Oligonucleotides

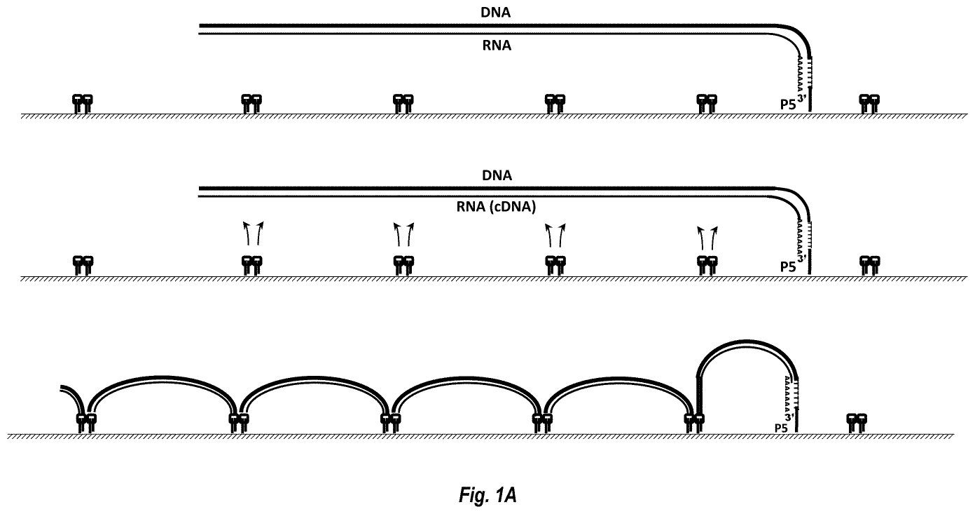

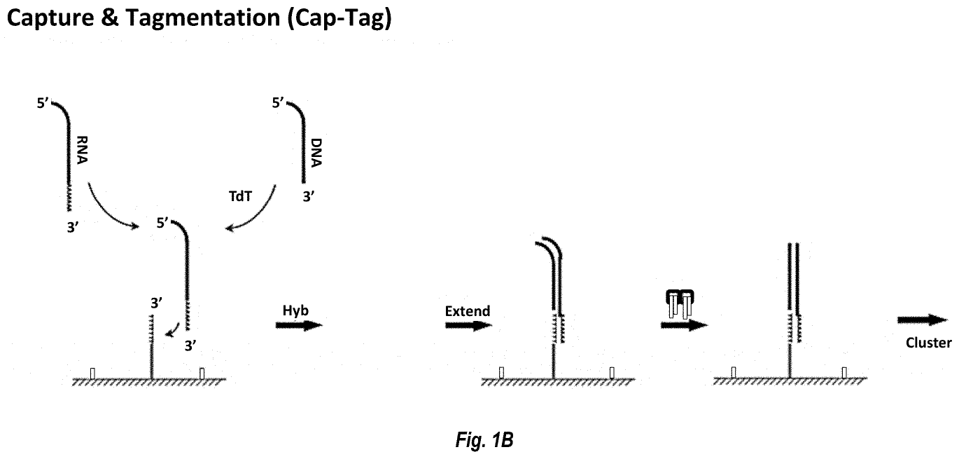

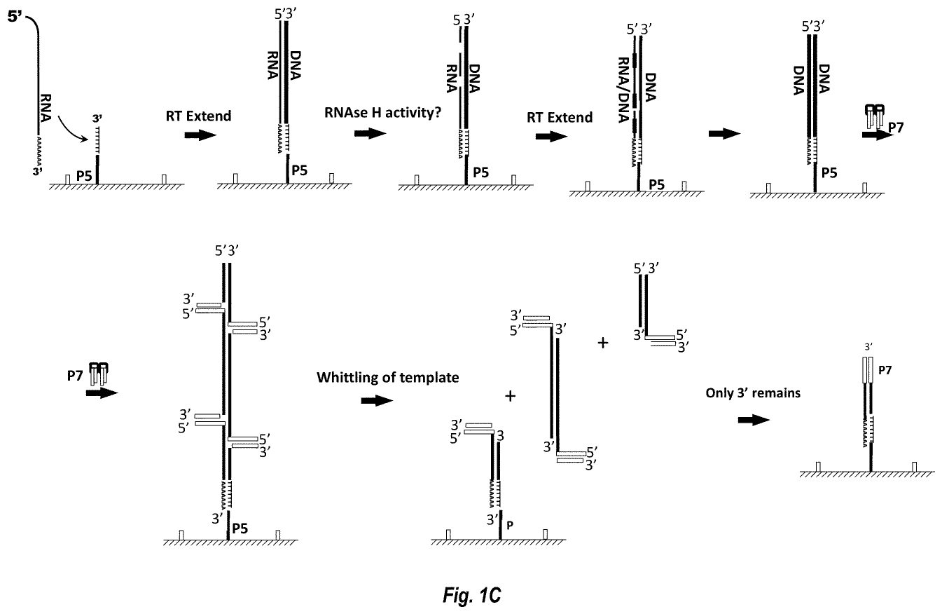

Many protocols for sequencing RNA samples employ a sample preparation that converts the RNA in the sample into a double-stranded cDNA format prior to sequencing. Provided herein are methods for sequencing RNA samples that uses DNA:RNA duplexes and that avoids a 3′ bias when tagmenting mRNA. In some embodiments, the present methods allow even coverage of the 5′ to 3′ of the target RNA in the resulting library products (as shown in FIG. 1A).

As used herein, a “DNA:RNA” duplex refers to a duplex of RNA and DNA. A DNA:RNA duplex encompasses RNA with some amount of DNA associated with it. The DNA of the DNA:RNA duplex allows fragmentation, i.e., the DNA in the DNA:RNA duplex is sufficient for the transposase to fragment the DNA:RNA duplex.

It is understood for all embodiments that a DNA:RNA duplex may also be converted to double-stranded DNA before fragmenting (i.e., performing tagmentation). In some embodiments, all of part of a second strand of cDNA is generated before fragmentation. In some embodiments, a DNA:RNA duplex may be converted to double-stranded DNA in solution or after a DNA:RNA duplex has been bound to an BLT. By converted to double-stranded DNA, it is meant that some or all of the RNA in a DNA:RNA duplex is converted to DNA. In such embodiments, fragmentation may occur in regions of double-stranded DNA.

In some embodiments, a sample comprising one or more species of RNA is added to a surface, whereupon mRNA transcripts are captured via a capture oligonucleotide, such as being captured by their 3′ polyA tails on the surface of the bead. A reverse transcriptase (RT) polymerase is then added to generate a first strand cDNA. Thus, in this method cDNA synthesis may be performed after RNA is bound to a capture oligonucleotide. The DNA:RNA duplex is tagmented by the surface-bound transposomes, thus generating library templates from the full length of the mRNA transcript.

In some embodiments, method of preparing an immobilized library of tagged DNA:RNA fragments from target RNA comprises:
applying a sample comprising target RNA to a solid support having transposome complexes and capture oligonucleotides immobilized thereon,
wherein the transposome complexes comprise a transposase bound to a first polynucleotide comprising a 3′ portion comprising a transposon end sequence, and a first tag;
wherein the sample is applied to the solid support under conditions wherein the 3′ end of the target RNA binds to the capture oligonucleotides;
adding a reverse transcriptase polymerase under conditions to synthesize cDNA and generate immobilized DNA:RNA duplexes on the capture oligonucleotides; and
fragmenting the DNA:RNA duplexes with the transposome complexes under conditions wherein the DNA:RNA duplexes are tagged on the 5′ end of one strand, thereby producing an immobilized library of DNA:RNA fragments wherein at least one strand is 5′-tagged with the first tag. In some embodiments, the 5′ end of one strand is the 5′ end of the RNA strand. In some embodiments, the 5′ end of one strand is the 5′ end of the DNA strand.

In some embodiments, activatable BLTs may be used, wherein transposases are attached to a first polynucleotide during the method. In some embodiments, transposases are attached to a first polynucleotide before or after adding a reverse transcriptase. In other words, transposases may be added after DNA:RNA duplexes are generated or before DNA:RNA duplexes are generated.

In some embodiments, a method of preparing an immobilized library of tagged DNA:RNA fragments from target RNA comprises applying a sample comprising target RNA to a solid support having capture oligonucleotides and a first polynucleotide immobilized thereon, wherein the first polynucleotide comprises a 3′ portion comprising a transposon end sequence and a first tag; wherein the sample is applied to the solid support under conditions wherein the 3′ end of the target RNA binds to the capture oligonucleotides; adding a transposase under conditions wherein the transposase binds to the first polynucleotide to form a transposome complex; adding a reverse transcriptase polymerase under conditions to synthesize cDNA and generate immobilized DNA:RNA duplexes on the capture oligonucleotides; and fragmenting the DNA:RNA duplexes with the transposome complexes under conditions wherein the DNA:RNA duplexes are tagged on the 5′ end of one strand, thereby producing an immobilized library of DNA:RNA fragments wherein at least one strand is 5′-tagged with the first tag.

In some embodiments, a method of preparing an immobilized library of tagged DNA:RNA fragments from target RNA comprises
applying a sample comprising target RNA to a solid support having capture oligonucleotides and a first polynucleotide immobilized thereon, wherein the first polynucleotide comprises a 3′ portion comprising a transposon end sequence and a first tag; wherein the sample is applied to the solid support under conditions wherein the 3′ end of the target RNA binds to the capture oligonucleotides; adding a reverse transcriptase polymerase under conditions to synthesize cDNA and generate immobilized DNA:RNA duplexes on the capture oligonucleotides;
adding a transposase under conditions wherein the transposase binds to the first polynucleotide to form a transposome complex; and
fragmenting the DNA:RNA duplexes with the transposome complexes under conditions wherein the DNA:RNA duplexes are tagged on the 5′ end of one strand, thereby producing an immobilized library of DNA:RNA fragments wherein at least one strand is 5′-tagged with the first tag.

In some embodiments, the method further comprising washing the solid support to remove any unbound target RNA after applying the sample to the solid support.

When DNA:RNA duplexes are added to the solid support, the transposome complexes will tagment the duplexes, thus generating fragments coupled at both ends to the surface. In some embodiments, the length of bridged fragments can be varied by changing the density of the transposome complexes on the surface. In certain embodiments, the length of the resulting bridged fragments is less than or equal to 100 bp, 200 bp, 300 bp, 400 bp, 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2100 bp, 2200 bp, 2300 bp, 2400 bp, 2500 bp, 2600 bp, 2700 bp, 2800 bp, 2900 bp, 3000 bp, 3100 bp, 3200 bp, 3300 bp, 3400 bp, 3500 bp, 3600 bp, 3700 bp, 3800 bp, 3900 bp, 4000 bp, 4100 bp, 4200 bp, 4300 bp, 4400 bp, 4500 bp, 4600 bp, 4700 bp, 4800 bp, 4900 bp, 5000 bp, 10000 bp, 30000 bp, or 100,000 bp. In such embodiments, the bridged fragments can then be amplified into clusters using standard cluster chemistry, as exemplified by the disclosure of U.S. Pat. Nos. 7,985,565 and 7,115,400, the contents of each of which is incorporated herein by reference in its entirety.

In some embodiments, fragmenting produces double-stranded DNA:RNA duplexes bridged to two immobilized transposome complexes on the solid support. In some embodiments, the length of the bridged duplexes is from 100 base pairs to 1500 base pairs.

In some embodiments, the DNA:RNA duplex generated from the 3′ end of the mRNA is attached at one end to the capture oligonucleotide and to an immobilized transposome complex at the other end. In some embodiments, a capture oligonucleotide comprises similar or the same sequences as those comprised in one or more transposon ends. In some embodiments, a capture oligonucleotide may comprise a first tag.

In some embodiments, the fragments of the DNA:RNA duplexes can be used to generate sequences of coding, untranslated region (UTR), introns, and/or intergenic sequences of the target RNA.

In some embodiments, an in vitro transposition reaction to tag the target DNA:RNA duplexes and to generate immobilized tagged DNA:RNA duplexes involves a transposase, a transposon sequence composition, and suitable reaction conditions.

As described herein, certain modifications to protocols may improve yield. However, these specific methods are not meant to limit the scope of the claims, but to provide guidance to one skilled in the art wanting to optimize methods for their particular sample. For example, a user working with samples comprising single cells may want to use methods described herein to improve library yield, as single cells would have limited amounts of RNA and DNA.

A. Samples and Target mRNA

In some embodiments, a sample comprises target RNA. In some embodiments, the sample comprises RNA and DNA. In some embodiments, the target RNA is mRNA. In some embodiments, the target RNA comprises coding, untranslated region (UTR), introns, and/or intergenic sequences.

In some embodiments, the step of applying a sample comprising target RNA or a sample comprising RNA and DNA comprises adding a biological sample to said solid support. The biological sample can be any type that comprises RNA or RNA and DNA and which can be deposited onto the solid surface for tagmentation. For example, the sample can comprise RNA or RNA and DNA in a variety of states of purification, including purified RNA or RNA and DNA. However, the sample need not be completely purified, and can comprise, for example, RNA or RNA and DNA mixed with protein, other nucleic acid species, other cellular components and/or any other contaminant. In some embodiments, the biological sample comprises a mixture of RNA or RNA and DNA, protein, other nucleic acid species, other cellular components and/or any other contaminant present in approximately the same proportion as found in vivo. For example, in some embodiments, the components are found in the same proportion as found in an intact cell. In some embodiments, the biological sample has a 260/280 absorbance ratio of less than or equal to 2.0, 1.9, 1.8, 1.7, 1.6, 1.5, 1.4, 1.3, 1.2, 1.1, 1.0, 0.9, 0.8, 0.7, or 0.60. In some embodiments, the biological sample has a 260/280 absorbance ratio of at least 2.0, 1.9, 1.8, 1.7, 1.6, 1.5, 1.4, 1.3, 1.2, 1.1, 1.0, 0.9, 0.8, 0.7, or 0.60. Because the methods provided herein allow RNA or RNA and DNA to be bound to solid supports, other contaminants can be removed merely by washing the solid support after surface bound tagmentation occurs. The biological sample can comprise, for example, a crude cell lysate or whole cells. For example, a crude cell lysate that is applied to a solid support in a method set forth herein, need not have been subjected to one or more of the separation steps that are traditionally used to isolate nucleic acids from other cellular components. Exemplary separation steps are set forth in Maniatis et al., Molecular Cloning: A Laboratory Manual, 2d Edition, 1989, and Short Protocols in Molecular Biology, ed. Ausubel, et al, hereby incorporated by reference.

In some embodiments, the sample that is applied to the solid support has a 260/280 absorbance ratio that is less than or equal to 1.7.

Thus, in some embodiments, the biological sample can comprise, for example, blood, plasma, serum, lymph, mucus, sputum, urine, semen, cerebrospinal fluid, bronchial aspirate, feces, and macerated tissue, or a lysate thereof, or any other biological specimen comprising RNA or RNA and DNA.

In some embodiments, the sample that is applied to the solid support is blood. In some embodiments, the sample that is applied to the solid support is a cell lysate.

In some embodiments, the cell lysate is a crude cell lysate. In some embodiments, the method further comprises lysing cells in the sample after applying the sample to the solid support to generate a cell lysate.

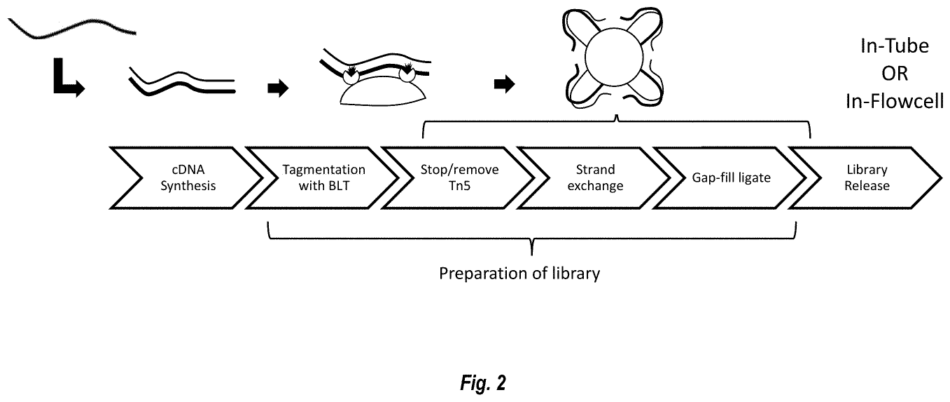

In some embodiments, DNA:RNA duplexes are prepared in solution and then immobilized to a BLT (See FIG. 2).

One advantage of the methods and compositions presented herein that a biological sample can be added to the flowcell and subsequent lysis and purification steps can all occur in the flowcell without further transfer or handling steps, simply by flowing the necessary reagents into the flowcell.

In some embodiments, the target RNA comprises a sequence complementary to at least a portion of one or more of the capture oligonucleotides.

In some embodiments, the target RNA is messenger RNA (mRNA), transfer RNA (tRNA), or ribosomal RNA (rRNA). Appropriate capture oligonucleotides could be designed based on the type of target RNA.

In some embodiments, the 3′ end of the target RNA binds to the capture oligonucleotides.

In some embodiments, the target RNA is mRNA. In some embodiments, the target RNA is polyadenylated (i.e. comprises a stretch of RNA that contains only adenine bases). In some embodiments, the mRNA comprise polyA tails. In some embodiments, the 3′ ends of the mRNA comprise polyA tails.

In some embodiments, the target mRNA comprises a polyA sequence and binds to capture oligonucleotides comprising polyT sequences.

The DNA and RNA comprised in a sample may be termed “total nucleic acid.” Methods described herein may be of use in preparing DNA and RNA libraries from a sample comprising total nucleic acid, wherein the fragments comprised in the DNA library each comprise a DNA-specific barcode and wherein the fragments comprised in the RNA library each comprise an RNA-specific barcode. Such methods may avoid the need to separate DNA and RNA prior to preparation of DNA and RNA libraries from a single sample.

B. Transposome Complexes

In some embodiments, a transposome complex comprises a transposase bound to one or more polynucleotide.

A “transposome complex” is comprised of at least one transposase (or other enzyme as described herein) and a transposon recognition sequence. In some such systems, the transposase binds to a transposon recognition sequence to form a functional complex that is capable of catalyzing a transposition reaction. In some respects, the transposon recognition sequence is a double-stranded transposon end sequence. The transposase binds to a transposase recognition site in a target nucleic acid and inserts the transposon recognition sequence into a target nucleic acid. In some such insertion events, one strand of the transposon recognition sequence (or end sequence) is transferred into the target nucleic acid, resulting in a cleavage event. Exemplary transposition procedures and systems that can be readily adapted for use with the transposases.

A “transposase” means an enzyme that is capable of forming a functional complex with a transposon end-containing composition (e.g., transposons, transposon ends, transposon end compositions) and catalyzing insertion or transposition of the transposon end- containing composition into a double-stranded target nucleic acid. A transposase as presented herein can also include integrases from retrotransposons and retroviruses.

Transposon based technology can be utilized for fragmenting DNA, wherein target nucleic acids, such as genomic DNA, are treated with transposome complexes that simultaneously fragment and tag the target (“tagmentation”), thereby creating a population of fragmented nucleic acid molecules tagged with unique adaptor sequences at the ends of the fragments. Tagmentation includes the modification of DNA by a transposome complex comprising transposase enzyme complexed with one or more tag (such as adaptor sequences) comprising transposon end sequences (referred to herein as transposons). Tagmentation thus can result in the simultaneous fragmentation of the DNA and ligation of the adaptors to the 5′ ends of both strands of duplex fragments.

A transposition reaction is a reaction wherein one or more transposons are inserted into target nucleic acids at random sites or almost random sites. Components in a transposition reaction may include a transposase (or other enzyme capable of fragmenting and tagging a nucleic acid as described herein, such as an integrase) and a transposon element that includes a double-stranded transposon end sequence that binds to the enzyme, and an adaptor sequence attached to one of the two transposon end sequences. One strand of the double-stranded transposon end sequence is transferred to one strand of the target nucleic acid and the complementary transposon end sequence strand is not (i.e., a non-transferred transposon sequence). The adaptor sequence can comprise one or more functional sequences (e.g., primer sequences) as needed or desired.

Exemplary transposases that can be used with certain embodiments provided herein include (or are encoded by): Tn5 transposase, Sleeping Beauty (SB) transposase, Vibrio harveyi, MuA transposase and a Mu transposase recognition site comprising R1 and R2 end sequences, Staphylococcus aureus Tn552, Ty1, Tn7 transposase, Tn/O and IS10, Mariner transposase, Tc1, P Element, Tn3, bacterial insertion sequences, retroviruses, and retrotransposon of yeast. More examples include IS5, Tn10, Tn903, IS911, and engineered versions of transposase family enzymes. The methods described herein could also include combinations of transposases, and not just a single transposase.

In some embodiments, the transposase is a Tn5, Tn7, MuA, or Vibrio harveyi transposase, or an active mutant thereof. In other embodiments, the transposase is a Tn5 transposase or a mutant thereof. In other embodiments, the transposase is a Tn5 transposase or a mutant thereof. In other embodiments, the transposase is a Tn5 transposase or an active mutant thereof. In some embodiments, the Tn5 transposase is a hyperactive Tn5 transposase, or an active mutant thereof. In some aspects, the Tn5 transposase is a Tn5 transposase as described in PCT Publ. No. WO2015/160895, which is incorporated herein by reference. In some aspects, the Tn5 transposase is a hyperactive Tn5 with mutations at positions 54, 56, 372, 212, 214, 251, and 338 relative to wild-type Tn5 transposase. In some aspects, the Tn5 transposase is a hyperactive Tn5 with the following mutations relative to wild-type Tn5 transposase: E54K, M56A, L372P, K212R, P214R, G251R, and A338V. In some embodiments, the Tn5 transposase is a fusion protein. In some embodiments, the Tn5 transposase fusion protein comprises a fused elongation factor Ts (Tsf) tag. In some embodiments, the Tn5 transposase is a hyperactive Tn5 transposase comprising mutations at amino acids 54, 56, and 372 relative to the wild type sequence. In some embodiments, the hyperactive Tn5 transposase is a fusion protein, optionally wherein the fused protein is elongation factor Ts (Tsf). In some embodiments, the recognition site is a Tn5-type transposase recognition site (Goryshin and Reznikoff, J. Biol. Chem., 273:7367, 1998). In one embodiment, a transposase recognition site that forms a complex with a hyperactive Tn5 transposase is used (e.g., EZ-Tn5TM Transposase, Epicentre Biotechnologies, Madison, Wis.). In some embodiments, the Tn5 transposase is a wild-type Tn5 transposase.

In some embodiments, the transposome complex comprises a dimer of two molecules of a transposase. In some embodiments, the transposome complex is a homodimer, wherein two molecules of a transposase are each bound to first and second transposons of the same type (e.g., the sequences of the two transposons bound to each monomer are the same, forming a “homodimer”). In some embodiments, the compositions and methods described herein employ two populations of transposome complexes. In some embodiments, the transposases in each population are the same. In some embodiments, the transposome complexes in each population are homodimers, wherein the first population has a first adaptor sequence in each monomer and the second population has a different adaptor sequence in each monomer.

The term “transposon end” refers to a double-stranded nucleic acid DNA that exhibits only the nucleotide sequences (the “transposon end sequences”) that are necessary to form the complex with the transposase or integrase enzyme that is functional in an in vitro transposition reaction. In some embodiments, a transposon end is capable of forming a functional complex with the transposase in a transposition reaction. As non-limiting examples, transposon ends can include the 19-bp outer end (“OE”) transposon end, inner end (“IE”) transposon end, or “mosaic end” (“ME”) transposon end recognized by a wild-type or mutant Tn5 transposase, or the R1 and R2 transposon end as set forth in the disclosure of US 2010/0120098, the content of which is incorporated herein by reference in its entirety. Transposon ends can comprise any nucleic acid or nucleic acid analogue suitable for forming a functional complex with the transposase or integrase enzyme in an in vitro transposition reaction. For example, the transposon end can comprise DNA, RNA, modified bases, non-natural bases, modified backbone, and can comprise nicks in one or both strands. Although the term “DNA” is used throughout the present disclosure in connection with the composition of transposon ends, it should be understood that any suitable nucleic acid or nucleic acid analogue can be utilized in a transposon end.

The term “transferred strand” refers to the transferred portion of both transposon ends. Similarly, the term “non-transferred strand” refers to the non-transferred portion of both “transposon ends.” The 3′-end of a transferred strand is joined or transferred to target DNA in an in vitro transposition reaction. The non-transferred strand, which exhibits a transposon end sequence that is complementary to the transferred transposon end sequence, is not joined or transferred to the target DNA in an in vitro transposition reaction.

In some embodiments, the transferred strand and non-transferred strand are covalently joined. For example, in some embodiments, the transferred and non-transferred strand sequences are provided on a single oligonucleotide, e.g., in a hairpin configuration. As such, although the free end of the non-transferred strand is not joined to the target DNA directly by the transposition reaction, the non-transferred strand becomes attached to the DNA fragment indirectly, because the non-transferred strand is linked to the transferred strand by the loop of the hairpin structure. Additional examples of transposome structure and methods of preparing and using transposomes can be found in the disclosure of US 2010/0120098, the content of which is incorporated herein by reference in its entirety.

In some embodiments, the transposome complexes comprise a transposase bound to a first polynucleotide. In some embodiments, the first polynucleotide comprises a 3′ portion comprising a transposon end sequence and a first tag.

In some embodiments, the transposome complexes comprise a second polynucleotide comprising a region complementary to the transposon end sequence.

1. Transposases

As used throughout, the term transposase refers to an enzyme that is capable of forming a functional complex with a transposon-containing composition (e.g., transposons, transposon compositions) and catalyzing insertion or transposition of the transposon-containing composition into the double-stranded target nucleic acid with which it is incubated in an in vitro transposition reaction. A transposase of the provided methods also includes integrases from retrotransposons and retroviruses. Exemplary transposases that can be used in the provided methods include wild-type or mutant forms of Tn5 transposase and MuA transposase.

A “transposition reaction” is a reaction wherein one or more transposons are inserted into target nucleic acids at random sites or almost random sites. Essential components in a transposition reaction are a transposase and DNA oligonucleotides that exhibit the nucleotide sequences of a transposon, including the transferred transposon sequence and its complement (i.e., the non-transferred transposon end sequence) as well as other components needed to form a functional transposition or transposome complex. The method of this disclosure is exemplified by employing a transposition complex formed by a hyperactive Tn5 transposase and a Tn5-type transposon end or by a MuA or HYPERMu transposase and a Mu transposon end comprising R1 and R2 end sequences (See e.g., Goryshin, I. and Reznikoff, W. S., J. Biol. Chem., 273: 7367, 1998; and Mizuuchi, Cell, 35: 785, 1983; Savilahti, H, et al., EMBO J., 14: 4893, 1995; which are incorporated by reference herein in their entireties). However, any transposition system that is capable of inserting a transposon end in a random or in an almost random manner with sufficient efficiency to tag target nucleic acids for its intended purpose can be used in the provided methods. Other examples of known transposition systems that could be used in the provided methods include but are not limited to Staphylococcus aureus Tn552, Ty1, Transposon Tn7, Tn/O and IS 10, Mariner transposase, Tel, P Element, Tn3, bacterial insertion sequences, retroviruses, and retrotransposon of yeast (See, e.g., Colegio O R et al, J. Bacteriol., 183: 2384-8, 2001; Kirby C et al, Mol. Microbiol., 43: 173-86, 2002; Devine S E, and Boeke J D., Nucleic Acids Res., 22: 3765-72, 1994; International Patent Application No. WO 95/23875; Craig, N L, Science. 271: 1512, 1996; Craig, N L, Review in: Curr Top Microbiol Immunol., 204: 27-48, 1996; Kleckner N, et al., Curr Top Microbiol Immunol., 204: 49-82, 1996; Lampe D J, et al., EMBO J., 15: 5470-9, 1996; Plasterk R H, Curr Top Microbiol Immunol, 204: 125-43, 1996; Gloor, G B, Methods Mol. Biol, 260: 97-1 14, 2004; Ichikawa H, and Ohtsubo E., J Biol. Chem. 265: 18829-32, 1990; Ohtsubo, F and Sekine, Y, Curr. Top. Microbiol. Immunol. 204: 1-26, 1996; Brown P O, et al, Proc Natl Acad Sci USA, 86: 2525-9, 1989; Boeke J D and Corces V G, Annu Rev Microbiol. 43: 403-34, 1989; which are incorporated herein by reference in their entireties).

The method for inserting a transposon into a target sequence can be carried out in vitro using any suitable transposon system for which a suitable in vitro transposition system is available or can be developed based on knowledge in the art. In general, a suitable in vitro transposition system for use in the methods of the present disclosure requires, at a minimum, a transposase enzyme of sufficient purity, sufficient concentration, and sufficient in vitro transposition activity and a transposon with which the transposase forms a functional complex with the respective transposase that is capable of catalyzing the transposition reaction. Suitable transposase transposon end sequences that can be used include but are not limited to wild-type, derivative or mutant transposon end sequences that form a complex with a transposase chosen from among a wild- type, derivative or mutant form of the transposase.

In some embodiments, the transposase comprises a Tn5 transposase. In some embodiments, the Tn5 transposase is hyperactive Tn5 transposase. In some embodiments, the transposase has increased activity for DNA:RNA duplexes, as compared to Tn5.

2. Deactivated Transposomes

In some embodiments, a transposome complex is reversibly deactivated during the method. In some embodiments, a transposome complex is reversibly deactivated in when the sample comprising target RNA is applied to the solid support having transposomes complexes and capture oligonucleotides and activated before or when fragmenting DNA:RNA complexes. In some embodiments, the transposome complex is activated before or when fragmenting by removing the transposome deactivator. Any of the methods of reversible deactivation can be used with the methods described herein comprising fragmenting of either DNA:RNA duplexes or double-stranded DNA. In other words, any transposome complex described herein may be reversibly deactivated. In some embodiments, performing tagmentation comprises activating a transposome complex that was previously in a reversibly deactivated state.

In some embodiments, the transposome complex is reversibly deactivated by a transposome deactivator bound to the transposome complex. In some embodiments, the transposome deactivator is bound to a Tn5 binding site of the transposome complex.

A wide range of means could deactivate a transposome. In some embodiments, the transposome deactivator comprises dephosphorylated ME′, extra bases, inhibitor duplexes, and/or heat-labile antibodies.

In some embodiments, a dephosphorylated ME′ sequence can be phosphorylated to generate phosphorylated ME′ sequence and activate the transposome.

In some embodiments, extra bases (i.e., nucleotides) are adjacent to the transposon end sequence to block association with the transposase. In some embodiments, the extra bases are separated from the transposon end sequence by a cleavable linker. In some embodiments, treatment with an agent to cleave the cleavable linker generates an active transposome complex.

In some embodiments, inhibitor duplexes are bound to the transposase of the transposome complex.

In some embodiments, a heat-labile antibody is complexes to the DNA binding site the transposase of the transposome complex. In some embodiments, the reaction solution comprising a deactivated transposome comprising a heat-labile antibody is heated, such that the heat-labile antibody is inactivated. Once the heat-labile antibody is inactivated, the transposome can be activated.

3. Solution-phase Transposome Complexes

The method presented herein can further comprise a step of providing transposome complexes in solution and contacting the solution-phase transposome complexes with the immobilized fragments under conditions whereby the DNA:RNA is fragmented by the transposome complexes solution; thereby obtaining immobilized nucleic acid fragments having one end in solution. In some embodiments, the transposome complexes in solution can comprise a second tag, such that the method generates immobilized nucleic acid fragments having a second tag, the second tag in solution. The first and second tags can be different or the same.

In some embodiments, the method further comprises contacting solution- phase transposome complexes with the immobilized DNA:RNA fragments under conditions whereby the DNA:RNA fragments are further fragmented by the solution-phase transposome complexes; thereby obtaining immobilized nucleic acid fragments having one end in solution.

In some embodiments, the solution-phase transposome complexes comprise a second tag, thereby generating immobilized nucleic acid fragments having a second tag in solution. In some embodiments, the first and second tags are different. In some embodiments, at least 50%, 55%, 60%, 65%, 70%, 75%, 80%, 85%, 90%, 95%, 96%, 97%, 98%, or 99% of the solution-phase transposome complexes comprise a second tag.

In some embodiments, one form of surface bound transposome is predominantly present on the solid support. For example, in some embodiments, at least 50%, 55%, 60%, 65%, 70%, 75%, 80%, 85%, 90%, 95%, 96%, 97%, 98%, or 99% of the tags present on said solid support comprise the same tag domain. In such embodiments, after an initial tagmentation reaction with surface bound transposomes, at least 50%, 55%, 60%, 65%, 70%, 75%, 80%, 85%, 90%, 95%, 96%, 97%, 98%, or 99% of the bridge structures comprise the same tag domain at each end of the bridge. A second tagmentation reaction can be performed by adding transposomes from solution that further fragment the bridges. In some embodiments, most or all of the solution phase transposomes comprise a tag domain that differs from the tag domain present on the bridge structures generated in the first tagmentation reaction. For example, in some embodiments, at least 50%, 55%, 60%, 65%, 70%, 75%, 80%, 85%, 90%, 95%, 96%, 97%, 98%, or 99% of the tags present in the solution phase transposomes comprise a tag domain that differs from the tag domain present on the bridge structures generated in the first tagmentation reaction.

In some embodiments, the length of the templates is longer than what can be suitably amplified using standard cluster chemistry. For example, in some embodiments, the length of templates is at least 100 bp, 200 bp, 300 bp, 400 bp, 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2100 bp, 2200 bp, 2300 bp, 2400 bp, 2500 bp, 2600 bp, 2700 bp, 2800 bp, 2900 bp, 3000 bp, 3100 bp, 3200 bp, 3300 bp, 3400 bp, 3500 bp, 3600 bp, 3700 bp, 3800 bp, 3900 bp, 4000 bp, 4100 bp, 4200 bp, 4300 bp, 4400 bp, 4500 bp, 4600 bp, 4700 bp, 4800 bp, 4900 bp, 5000 bp, 10000 bp, 30000 bp or 100,000 bp. In such embodiments, then a second tagmentation reaction can be performed by adding transposomes from solution that further fragment the bridges, as described in U.S. Pat. No. 9,683,230, which is incorporated herein in its entirety. The second tagmentation reaction can thus remove the internal span of the bridges, leaving short stumps anchored to the surface that can converted into clusters ready for further sequencing steps. In particular embodiments, the length of the template can be within a range defined by an upper and lower limit selected from those exemplified above.

In some embodiments, transposition in solution (i.e., with solution-phase transposome complexes) may be used to incorporate a sequence that can hybridize to a capture probe. In this way, tagmentation can be used to generate fragments that can bind specifically to a solid support comprising capture probes on its surface. In some embodiments, cDNA or DNA:RNA duplexes generated from RNA are tagmented in solution to incorporate a tag comprising a sequence that can hybridize to capture probes. After this tagmentation, the fragments generated from the cDNA or DNA:RNA duplexes can be bound to a solid support comprising capture probes. In some embodiments, the capture probes comprise P5 or P7 sequences (or their complements).

C. Beads Comprising Capture Oligonucleotides

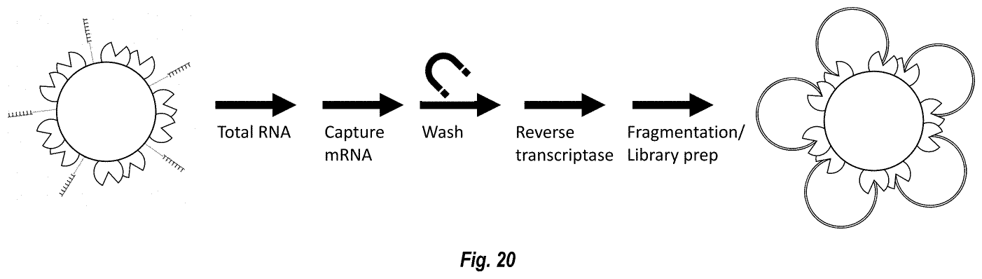

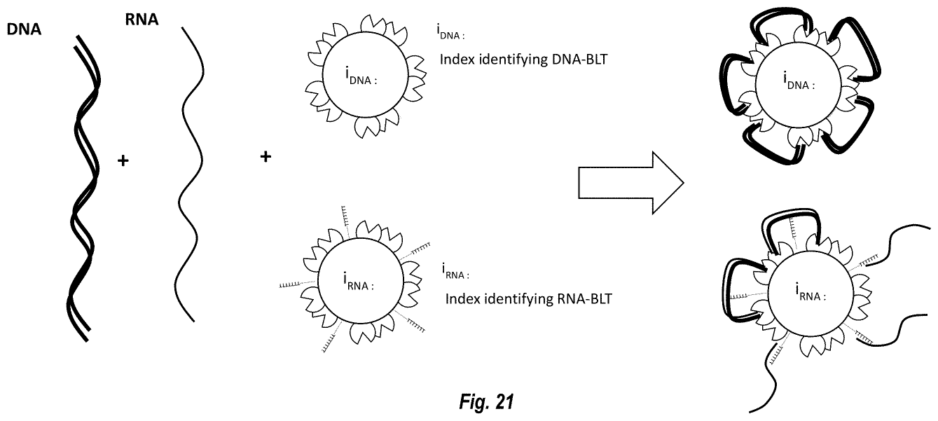

In some embodiments, capture oligonucleotides are immobilized on a solid support. In some embodiments, the 3′ end of the target RNA binds to the capture oligonucleotides. In some embodiments, capture oligonucleotides may serve to immobilize the target RNA on the solid support. An exemplary workflow using a bead comprising a capture oligonucleotide to capture mRNA is shown in FIG. 20.

In some embodiments, the capture oligonucleotides comprise a polyT sequence.

In some embodiments, the target RNA is mRNA, and the mRNA binds to capture oligonucleotides comprising polyT sequences.

In some embodiments, the capture oligonucleotides do not comprise polyT sequences.

In some embodiments, the capture oligonucleotides are immobilized to the beads via P5 or P7 sequences.

In some embodiments, the capture oligonucleotides further comprise a bead code or other type of barcode. As used herein, a “bead code” is a nucleic acid sequence that is present on a bead and which is different from all or most other beads in a pool of beads. In some embodiments, a bead code can be used to identify fragments that are generated on the same bead.

In some embodiments, sequences comprised in the capture oligonucleotide are incorporated into cDNA during synthesis.

In some embodiments, the capture oligonucleotides comprise a tag that is also present in the first tag comprised in the first polynucleotide of the immobilized transposomes.

In some embodiments, a solid support does not comprise a capture oligonucleotide for immobilizing target nucleic acid. For example, double-stranded DNA and DNA:RNA duplexes can be immobilized on solid support via binding to immobilized transposome complexes.

D. Polynucleotides and Immobilized Transposome Complexes

In some embodiments, the transposome complex composition comprises or consists of at least one transposon with one or more other nucleotide sequences in addition to the transposon sequences. Such nucleotide sequences may be referred to as polynucleotides.

In the methods and compositions presented herein, transposome complexes are immobilized to the solid support. In some embodiments, the transposome complexes are immobilized to the support via one or more polynucleotides, such as a polynucleotide comprising a transposon end sequence. In some embodiments, the transposome complex may be immobilized via a linker molecule coupling the transposase enzyme to the solid support. In some embodiments, both the transposase enzyme and the polynucleotide are immobilized to the solid support. When referring to immobilization of molecules (e.g. nucleic acids) to a solid support, the terms “immobilized” and “attached” are used interchangeably herein and both terms are intended to encompass direct or indirect, covalent or non-covalent attachment, unless indicated otherwise, either explicitly or by context. In some embodiments, covalent attachment may be used, but generally all that is required is that the molecules (e.g. nucleic acids) remain immobilized or attached to the support under the conditions in which it is intended to use the support, for example in applications requiring nucleic acid amplification and/or sequencing.

In some embodiments, the transposome complexes comprise a transposase bound to a first polynucleotide comprising a 3′ portion comprising a transposon end sequence and a first tag. In some embodiments, the first tag is a DNA-specific barcode.

In some embodiments, the transposome complexes comprise a transposase bound to a first polynucleotide comprising a 3′ portion comprising a transposon end sequence and a second tag. In some embodiments, the second tag is an RNA-specific barcode.

Thus, in some embodiments, the transposon composition comprises a transferred strand with one or more other nucleotide sequences 5′ of the transferred transposon sequence, e.g., a tag sequence. In addition to the transferred transposon sequence, the tag can have one or more other tag portions or tag domains.

“Tagmentation,” as used herein, refers to the use of transposase to fragment and tag nucleic acids. Tagmentation includes the modification of DNA by a transposome complex comprising transposase enzyme complexed with one or more tag (such as adaptor sequences) comprising transposon end sequences (referred to herein as transposons). Tagmentation thus can result in the simultaneous fragmentation of the DNA and ligation of the adaptors to the 5′ ends of both strands of duplex fragments.

In some embodiments, the transposome complex is immobilized to the solid support via the first polynucleotide.

In some embodiments, the transposome complexes comprise a second polynucleotide comprising a region complementary to the transposon end sequence. In some embodiments, the transposome complex is immobilized to the solid support via the second polynucleotide.

In some embodiments, the transposome complexes are present on the solid support at a density of at least 10³, 10⁴, 10⁵, or 10⁶ complexes per mm².

In some embodiments, the lengths of the double-stranded fragments in the immobilized library are adjusted by increasing or decreasing the density of transposome complexes on the solid support.

A number of different types of immobilized transposomes can be used in these methods, as described in U.S. Pat. No. 9,683,230, which is incorporated herein in its entirety.

E. Spatially Segregated Capture of Nucleic Acids on Beads

In some embodiments, a solid support comprising capture oligonucleotides is used to capture a nucleic acid. In some embodiments, the nucleic acid is RNA. In some embodiments, the RNA is mRNA. In some embodiments, a capture oligonucleotide comprising a polyT sequence is used to capture mRNA.

In some embodiments, RNA is captured on a capture bead. In some embodiments, RNA is captured on an RNA-BLT.

In some embodiments, RNA is captured on a solid support within a droplet. In some embodiments, applying a sample comprising target RNA to a solid support is performed in a droplet.

In some embodiments, DNA is captured on a solid support within a droplet. In some embodiments, applying a sample comprising target DNA to a solid support is performed in a droplet.

1. Droplets

In some embodiments, applying a sample comprising target RNA to a solid support comprises providing a single cell in a droplet together with a bead; lysing the cell in the droplet; releasing the target RNA from the single cell; and capturing the target RNA on the bead. In some embodiments, the droplet is removed before synthesizing cDNA. A variety of methods using droplets are known in the art, such Publications WO 2015/168161 and WO 2017/040306, each of which is incorporated herein in its entirety. Target DNA from a sample could be similarly captured on a bead (such as a BLT) and then the droplet is removed.

In some embodiments, after capture of RNA in droplets, cDNA synthesis is performed in bulk on the beads to generate a first strand of cDNA (i.e. to generate a DNA:RNA duplex). As used herein, “in bulk” is used to denote that steps are performed on beads, but these beads are in solution and not separated by droplets. Accordingly, when method steps are performed in bulk after capture of nucleic acids to beads, these immobilized nucleic acids are not in solution but remain immobilized on beads. In general, all methods described herein allow for capture of RNA or DNA in droplets, while later steps may either be performed in droplets or performed in bulk. In some embodiments, tagmentation is performed in bulk.

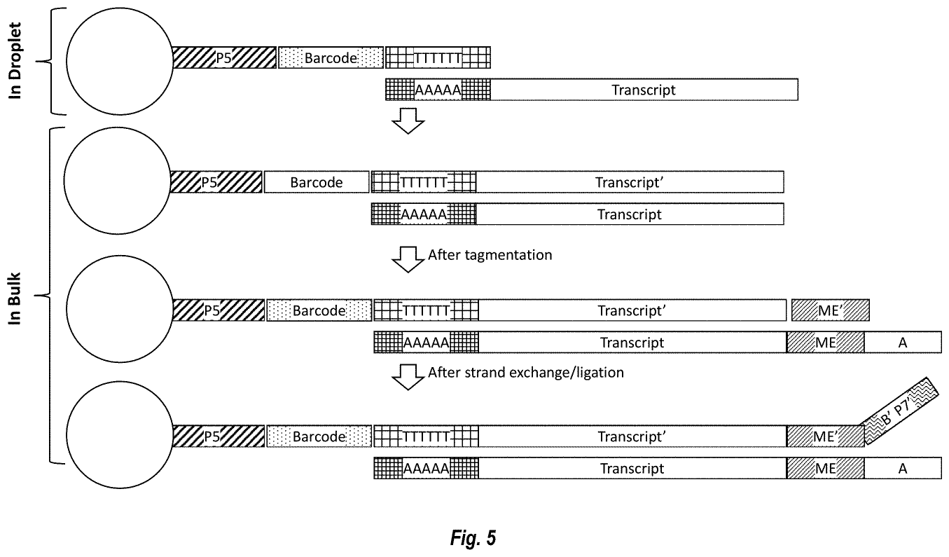

For example, FIG. 5 shows a representative example wherein a bead code (i.e., a barcode) is incorporated during synthesis of a first strand of cDNA to prepare a DNA:RNA duplex in a droplet. The bead code is comprised in a capture oligonucleotide also comprising a polyT sequence (to bind to poly-A tails of mRNA) and a P5 adapter sequence that can be used for immobilizing fragments on solid surfaces. The immobilized DNA:RNA duplex can be tagmented in bulk, followed by strand exchange and ligation. Adapters (such as B′ and P7′) can be added to the free end of the fragment (not attached to the bead), such as with tagmentation in solution or with PCR.

2. Barcoding in Droplets

In some embodiments, barcoding can be performed by segregating individual cells into droplets and incorporating a bead code. In some embodiments, a bead code can be incorporated into library fragments, based on a bead code comprised in a bead in a droplet. In some embodiments, droplets are used to spatially separate samples.

In some embodiments, BLTs comprise an immobilized oligonucleotide comprising a bead code. In some embodiments, the immobilized oligonucleotide comprises a first transposon comprising a bead code. In some embodiments, an immobilized oligonucleotide comprises a bead code and a hybridization sequence for binding to a second transposon.

In some embodiments, a bead code can be incorporated into a cDNA generated from an RNA from a sample. In some embodiments, a bead code can be incorporated into library fragments during tagmentation.

In some embodiments, the droplets are segregated from each other in an emulsion. In some embodiments, the droplets are formed and/or manipulated using a droplet actuator. In some embodiments, one or more droplets comprise a different set of barcode-containing first strand synthesis primers. In some embodiments, each droplet comprises a multitude of first strand synthesis primers, each of these primers have identical sequence including identical barcodes and the barcodes from one droplet differ from another droplet, while the remaining portion of the first strand synthesis primer remains the same between the droplets. Thus, in these embodiments, the barcodes act as identifier for the droplets as well as well as the single cell encompassed by the droplet.

In some embodiments, one or more droplets comprise a different set of UMI-containing first strand synthesis primers. Thus, each individual cell that is lysed in each droplet will be identifiable by the barcodes in each droplet. In some embodiments, droplet-based barcoding can be performed by merging droplets containing single cells with other droplets that comprise unique sets of barcodes. This format allows additional multiplexing beyond that available in a multiwall format. First strand synthesis and template switching are performed within each individual droplet. Additionally or alternatively, in some embodiments, droplets can be merged prior to tagmentation. Additionally or alternatively, in some embodiments, droplets can be merged after PCR and prior to tagmentation. For example, in some embodiments, after first strand synthesis is performed in individual droplets, the tagged cDNAs can be merged, thus pooling the cDNAs.

In some embodiments, sample and UMI barcoding can be performed by segregating individual cells with beads that bear a UMI and/or barcode-tagged primer for first strand synthesis. In some embodiments, beads are segregated into droplets in an emulsion. In some embodiments, beads are segregated and manipulated using a droplet actuator. In some embodiments, bead-based barcoding can be performed by creating a set of beads, each bead bearing a unique set or sets of barcodes.

3. Spatial Segregation Using Nucleic Acid Capture on BLT in Droplet

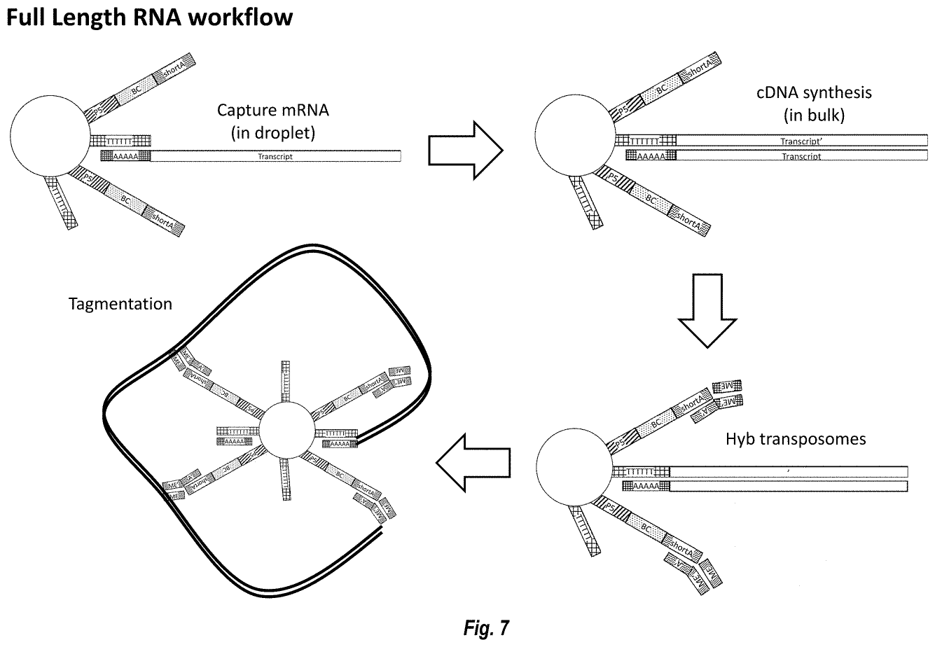

In some embodiments, RNA is captured on BLTs in droplets, and then cDNA preparation and fragmentation are performed before fragments are released from the BLTs. A cell and a capture bead can be co-encapsulated in a droplet, followed by lysis and mRNA capture in the droplet. Then, cDNA synthesis, tagmentation, and ligation can also be performed in bulk, with the resulting library fragments retained on the beads. A representative method is shown in FIG. 7, wherein the BLT is an activatable BLT and transposomes are assembled after cDNA synthesis in bulk.

In some embodiments, DNA is captured on BLTs in droplets, and fragmentation is performed before fragments are released from the BLTs.

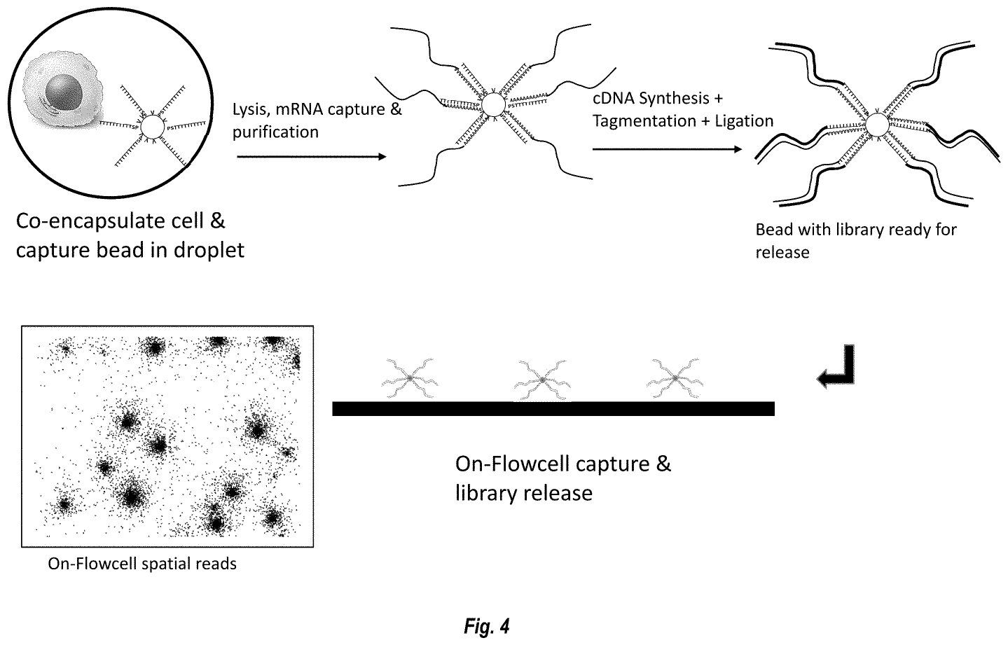

In some embodiments, library fragments are retained on beads due to the association of transposases (comprised in immobilized transposome complexes on BLTs) with fragments. In some embodiments, fragments remain immobilized on beads until a protease or SDS is added to release fragments from the transposases. In some embodiments, beads with immobilized library fragments are delivered to a solid support for sequencing (such as a flowcell) and the library is released from the bead. Such a release from beads captured on the flowcell would enable “on-flowcell spatial reads” as shown in FIG. 4, wherein fragments from a single bead will be released in close proximity to each other. In this way, fragments that are in close spatial proximity on the flowcell can be determined to have likely originated from nucleic acids from the same cell.

In some embodiments, capture of nucleic acid in droplets followed by library preparation on BLTs allows for segregation of fragments from different cells, even in the absence of barcoding.

4. Spatial Segregation Using a Compartmentalized Solid Support

In some embodiments, compartmentalization allows for spatial separation of library fragments for sequencing. In some embodiments, compartmentalization allows for fragments from a DNA or RNA in an original sample to be in close proximity after library preparations. In some embodiments, sequencing data and proximity data together can be used to determine fragments that were comprised in a starting DNA or RNA molecule in a sample.

In some embodiments, compartmentalization is performed using a solid support comprising microwell. In some embodiments, a single cell is compartmentalized in a microwell. In some embodiments, calculations of cell density and volume can be used to dilute a sample such that most cells are compartmentalized in a microwell that does not contain another cell.

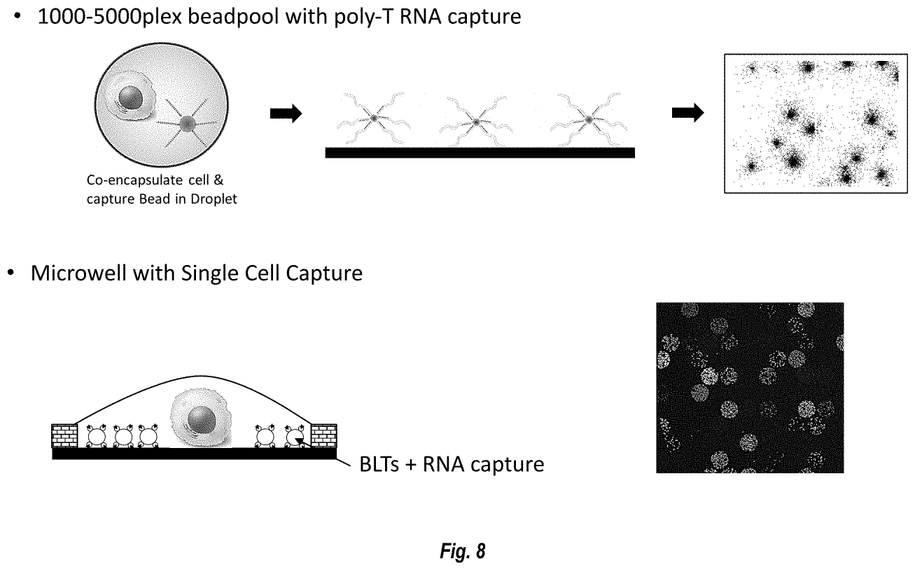

FIG. 8 shows a representative example, wherein poly-T RNA capture can be performed on a flowcell comprising microwells allowing for capture of a single cell per microwell.

In some embodiments, applying a sample comprising target RNA to a solid support is performed in a microwell on the solid support. In some embodiments, applying a sample comprising target RNA to a solid support comprises lysing a cell and releasing target RNA from the single cell in a microwell. In some embodiments, a method further comprises releasing an immobilized library of DNA:RNA fragments and sequencing the fragments in the same microwell. In this way, fragments that are localized to a given microwell can be characterized as coming from a single cell. In some embodiments, the sequencing data allows for the resolution of fragments that had been immobilized on the same solid support based on the spatial proximity of fragments on the surface for sequencing.

In some embodiments, the solid support is a flowcell comprising microwells.

In some embodiments, a flowcell comprising microwells contains a polymer coating. In some embodiments, the flowcell is coated with a covalently attached polymer. In some embodiments, the covalently attached polymer is PAZAM. In some embodiments, the polymer coating comprises reactive sites for reacting with oligonucleotides. Such covalently attached polymers are described in WO 2013/184796, which is incorporated by reference in its entirety herein. In some embodiments, a polymer such as PAZAM is crosslinked using ultraviolet light.

Methods with flowcells comprising microwells may also use specialized hybridization buffers and adapter blockers, as described in WO 2020/036991, which is incorporated by reference in its entirety herein.

F. Solid Supports

In the methods and compositions presented herein, transposome complexes are immobilized to the solid support. In some embodiments, the transposome complexes and/or capture oligonucleotides are immobilized to the support via one or more polynucleotides, such as a polynucleotide comprising a transposon end sequence. In some embodiments, the transposome complex may be immobilized via a linker molecule coupling the transposase enzyme to the solid support. In some embodiments, both the transposase enzyme and the polynucleotide are immobilized to the solid support. When referring to immobilization of molecules (e.g. nucleic acids) to a solid support, the terms “immobilized” and “attached” are used interchangeably herein and both terms are intended to encompass direct or indirect, covalent or non-covalent attachment, unless indicated otherwise, either explicitly or by context. In some embodiments, covalent attachment may be used, but generally all that is required is that the molecules (e.g. nucleic acids) remain immobilized or attached to the support under the conditions in which it is intended to use the support, for example in applications requiring nucleic acid amplification and/or sequencing.

Certain embodiments may make use of solid supports comprised of an inert substrate or matrix (e.g. glass slides, polymer beads etc.) which has been functionalized, for example by application of a layer or coating of an intermediate material comprising reactive groups which permit covalent attachment to biomolecules, such as polynucleotides. Examples of such supports include, but are not limited to, polyacrylamide hydrogels supported on an inert substrate such as glass, particularly polyacrylamide hydrogels as described in WO 2005/065814 and US 2008/0280773, the contents of which are incorporated herein in their entirety by reference. In such embodiments, the biomolecules (e.g. polynucleotides) may be directly covalently attached to the intermediate material (e.g. the hydrogel) but the intermediate material may itself be non-covalently attached to the substrate or matrix (e.g. the glass substrate). The term “covalent attachment to a solid support” is to be interpreted accordingly as encompassing this type of arrangement.

The terms “solid surface,” “solid support” and other grammatical equivalents herein refer to any material that is appropriate for or can be modified to be appropriate for the attachment of the transposome complexes. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon', etc.), polysaccharides, nylon or nitrocellulose, ceramics, resins, silica or silica-based materials including silicon and modified silicon, carbon, metals, inorganic glasses, plastics, optical fiber bundles, and a variety of other polymers. Particularly useful solid supports and solid surfaces for some embodiments are located within a flowcell apparatus. Exemplary flowcells are set forth in further detail below.

In some embodiments, the solid support comprises a patterned surface suitable for immobilization of transposome complexes in an ordered pattern. A “patterned surface” refers to an arrangement of different regions in or on an exposed layer of a solid support. For example, one or more of the regions can be features where one or more transposome complexes are present. The features can be separated by interstitial regions where transposome complexes are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. In some embodiments, the transposome complexes are randomly distributed upon the solid support. In some embodiments, the transposome complexes are distributed on a patterned surface. Exemplary patterned surfaces that can be used in the methods and compositions set forth herein are described in U.S. application Ser. No. 13/661,524 or US Pat. App. Publ. No. 2012/0316086 A1, each of which is incorporated herein by reference.

In some embodiments, the solid support comprises an array of wells or depressions in a surface. This may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The composition and geometry of the solid support can vary with its use. In some embodiments, the solid support is a planar structure such as a slide, chip, microchip and/or array. As such, the surface of a substrate can be in the form of a planar layer. In some embodiments, the solid support comprises one or more surfaces of a flowcell. The term “flowcell” as used herein refers to a chamber comprising a solid surface across which one or more fluid reagents can be flowed. Examples of flowcells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

In some embodiments, the solid support or its surface is non-planar, such as the inner or outer surface of a tube or vessel. In some embodiments, the solid support comprises microspheres or beads. By “microspheres” or “beads” or “particles” or grammatical equivalents herein is meant small discrete particles. Suitable bead compositions include, but are not limited to, plastics, ceramics, glass, polystyrene, methyl styrene, acrylic polymers, paramagnetic materials, thoria sol, carbon graphite, titanium dioxide, latex or cross-linked dextrans such as Sepharose, cellulose, nylon, cross-linked micelles and teflon, as well as any other materials outlined herein for solid supports may all be used. “Microsphere Selection Guide” from Bangs Laboratories, Fishers Ind. is a helpful guide. In certain embodiments, the microspheres are magnetic microspheres or beads.

The beads need not be spherical; irregular particles may be used. Alternatively or additionally, the beads may be porous. The bead sizes range from nanometers, i.e. 100 nm, to millimeters, i.e. 1 mm, with beads from 0.2 micron to 200 microns, or from 0.5 to 5 microns, although in some embodiments smaller or larger beads may be used.

The density of these surface bound transposomes can be modulated by varying the density of the first polynucleotide or by the amount of transposase added to the solid support. For example, in some embodiments, the transposome complexes are present on the solid support at a density of at least 10³, 10⁴, 10⁵, or 10⁶ complexes per mm².

Attachment of a nucleic acid to a support, whether rigid or semi-rigid, can occur via covalent or non-covalent linkage(s). Exemplary linkages are set forth in US Pat. Nos. 6,737,236; 7,259,258; 7,375,234 and 7,427,678; and US Pat. Pub. No. 2011/0059865 A1, each of which is incorporated herein by reference. In some embodiments, a nucleic acid or other reaction component can be attached to a gel or other semisolid support that is in turn attached or adhered to a solid-phase support. In such embodiments, the nucleic acid or other reaction component will be understood to be solid-phase.

In some embodiments, the solid support comprises microparticles, beads, a planar support, a patterned surface, or wells. In some embodiments, the planar support is an inner or outer surface of a tube.

In some embodiments, a solid support has a library of tagged RNA fragments immobilized thereon prepared. In some embodiments, a solid support has a library of tagged DNA fragments immobilized thereon prepared. In some embodiments, more than one solid support is used to generate a library of tagged RNA fragments on one solid support and a library of tagged DNA fragments on another.

In some embodiments, a solid support comprises capture oligonucleotides and a first polynucleotide immobilized thereon, wherein the first polynucleotide comprises a 3′ portion comprising a transposon end sequence and a first tag. In some embodiments, the first tag is an DNA-specific barcode. In some embodiments, this solid support is for tagmenting DNA and is termed a DNA bead-linked transposome (DNA BLT).

In some embodiments, the solid support further comprises a transposase bound to the first polynucleotide to form a transposome complex.

In some embodiments, a solid support comprises capture oligonucleotides and a second polynucleotide immobilized thereon, wherein the second polynucleotide comprises a 3′ portion comprising a transposon end sequence and a second tag. In some embodiments, the second tag is an RNA-specific barcode. In some embodiments, this solid support is for tagmenting nucleic acid generated from RNA (either ds-cDNA or DNA:RNA duplexes) and is termed an RNA bead- linked transposome (RNA BLT).

In some embodiments, the solid support further comprises a transposase bound to the second polynucleotide to form a transposome complex.

In some embodiments, solid supports comprise a library of tagged fragments immobilized thereon prepared according to any of the methods described herein.

In some embodiments, a kit comprises a solid support as described herein. In some embodiments, a kit further comprises a transposase. In some embodiments, a kit further comprises a reverse transcriptase polymerase. In some embodiments, a kit further comprises a second solid support for immobilizing DNA comprising a second transposome complex comprising a transposase and a third polynucleotide comprising a 3′ portion comprising a transposon end sequence, and optionally a second tag.

G. DNA BLTs and RNA BLTs

In some embodiments, these methods use BLTs (bead-linked transposomes). Transposomes bound to a surface (e.g., BLTs) can tagment long molecules of double-stranded DNA and making template libraries on beads or other surfaces (U.S. Pat. No. 9,683,230). Anchoring transposomes to beads gives novel properties such as controllable insert size and yield. This is the basis of the Illumina DNA Flex PCR-Free technology, previously known as Illumina's Nextera technology. BLTs that can tagment double-stranded DNA may be referred to as DNA-BLTs.

As transposon ends have affinity for DNA, DNA BLTs do not require a capture oligonucleotide for immobilization on beads, and instead DNA can be immobilized using polynucleotides comprising a transposon end sequence. Alternatively, a capture oligonucleotide may be used to capture DNA molecules.

In some embodiments, a solid support for immobilizing DNA comprises first transposome complexes immobilized thereon. In some embodiments, the first transposome complexes comprise a transposase and a first polynucleotide comprising a 3′ portion comprising a transposon end sequence. In some embodiments, the first polynucleotide further comprises a first tag. In some embodiments, the first tag is a DNA-specific barcode.

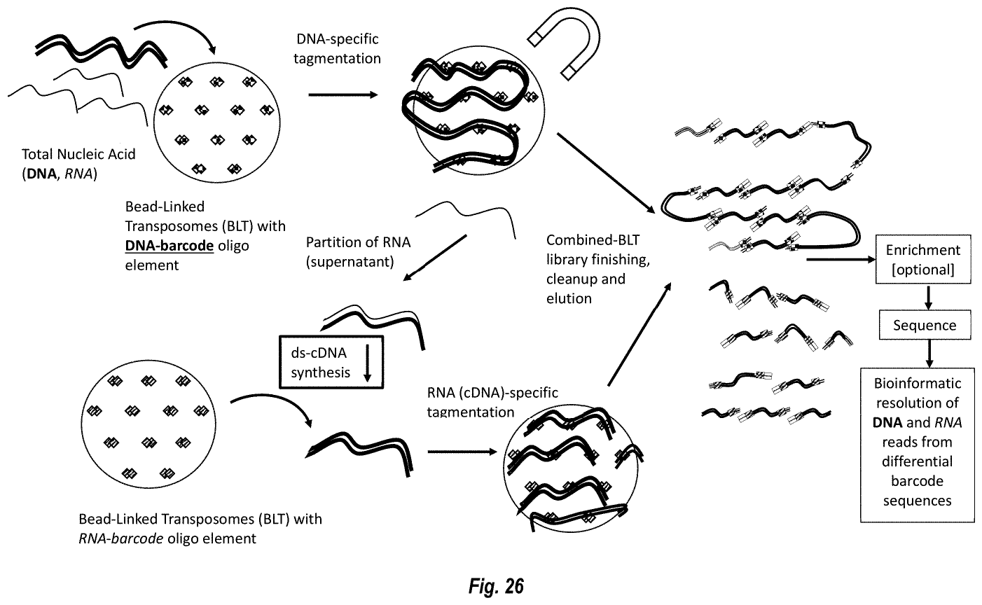

In some embodiments, these methods use RNA BLTs. As used herein “RNA BLTs” refer to BLTs used to prepare tagged fragments that originated from RNA in a sample. As such, an RNA BLTs may generate fragments from a nucleic acid that is generated from RNA. In some embodiments, RNA BLTs generate fragments from ds-cDNA generated from RNA (after synthesis of two stranded of cDNA). In some embodiments, RNA BLTs generate fragments from DNA:RNA duplexes (wherein only a single strand of cDNA is produced). In some embodiments, RNA BLTs serve to incorporate an RNA-specific barcode, while DNA BLTs serve to incorporate a DNA-specific barcode, as shown in FIGS. 26-28.

In some embodiments, a solid support for immobilizing a nucleic acid generated from RNA (such as ds-cDNA or DNA:RNA duplexes) comprises second transposome complexes immobilized thereon. In some embodiments, the second transposome complexes comprise a transposase and a first polynucleotide comprising a 3′ portion comprising a transposon end sequence. In some embodiments, this transposase has increased activity for generating fragments from DNA:RNA duplexes. In some embodiments, the first polynucleotide further comprises a second tag. In some embodiments, the second tag is an RNA-specific barcode.

1. Activatable BLTs

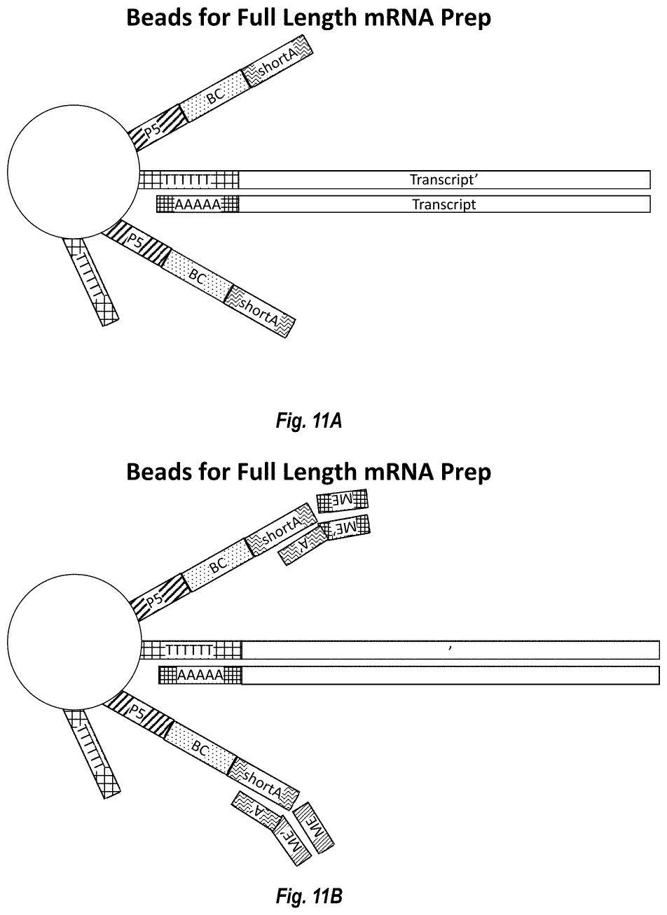

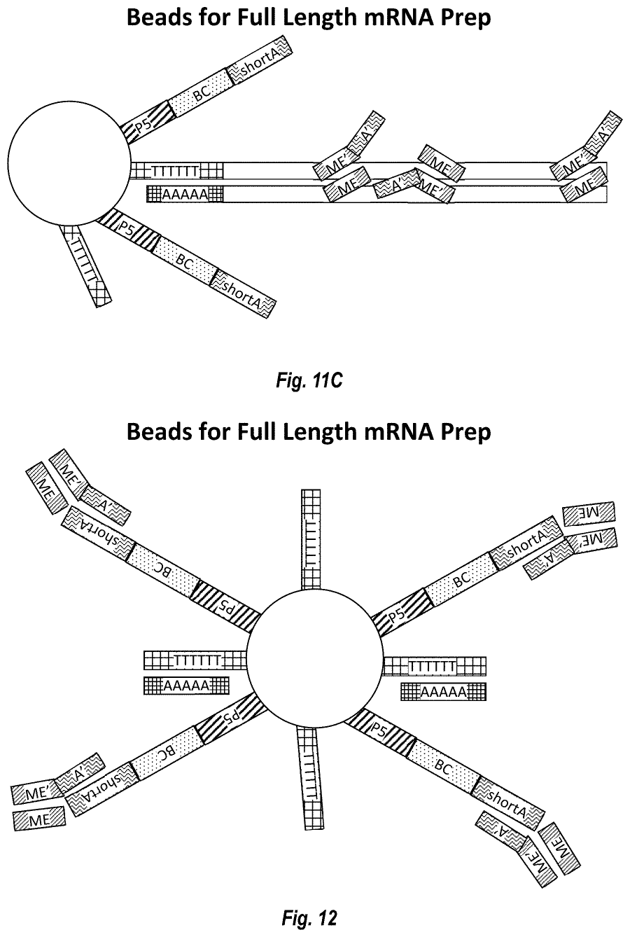

In some embodiments, beads can be used to allow for the user to generate active transposomes at the time of their choice. For examples, beads that allow for capturing transposomes from solution at a time of the user's choice may be termed “activatable BLTs.” A common aspect of activatable BLTs is that they may be applied to a sample in a state wherein they cannot fragment nucleic acid (such as DNA:RNA duplexes or double-stranded cDNA), but a user can activate the BLTs at a time of their choice. In this way, the user controls the timing of tagmentation in a multi-step method. A range of different types of activatable transposomes are described herein and can be used in different methods. FIGS. 11A-11C show beads that comprise capture oligonucleotides and oligonucleotides that can bind transposomes from solution via a ShortA sequence. FIG. 12 shows an embodiment wherein a first and second transposon have been bound to a bead via a ShortA sequence.

In some embodiments, activatable BLTs avoid undesired tagmentation. For example, use of activatable BLTs can allow a user to ensure that cDNA synthesis has been completed (such as generation of DNA:RNA duplexes) before tagmentation by transposomes occurs. In this way, the user can avoid fragmentation of “partial” DNA:RNA duplexes (i.e., tagmentation of incomplete DNA:RNA duplexes wherein cDNA has only been generated from a portion of the RNA).

Activatable BLTs have a number of advantages, such as allowing a user to control the timing of tagmentation in a multi-step method. In some embodiments, activatable BLTs can order steps of tagmentation of DNA from a sample versus cDNA or DNA:RNA duplexes generated from RNA from a sample. Such ordering can allow for tagging of fragments that originated from DNA versus those that originated from RNA.

Activatable BLTs can be used in any method described herein with transposomes immobilized on a solid support (such as a bead). In some embodiments, activatable BLTs are DNA-BLTs or RNA-BLTs.

In some embodiments, activatable RNA BLTs are for capturing RNA, preparing DNA:RNA duplexes, and then tagmenting the DNA:RNA duplexes. In some embodiments, activatable BLTs comprise an immobilized polyT sequence for capturing mRNA.

In some embodiments, a solid support comprises capture oligonucleotides and an immobilized oligonucleotide, wherein the immobilized oligonucleotide comprises a sequence for hybridizing to a hybridization sequence comprised in a second transposon comprised in a transposome complex. In some embodiments, the solid support is a bead. In some embodiments, the immobilized oligonucleotide further comprises a bead code and/or one or more adapter sequence. In some embodiments, the bead is comprised in a pool of beads, wherein each bead comprises an immobilized oligonucleotide comprising a different bead code as compared to the bead code comprised in immobilized oligonucleotides comprised in other beads in the pool.

In some embodiments, activatable BLTs comprise immobilized oligonucleotides that can capture transposomes from solution. In some embodiments, activatable BLTs comprise immobilized oligonucleotides comprising a hybridization sequence that can bind to a transposon comprised in a transposome complex.

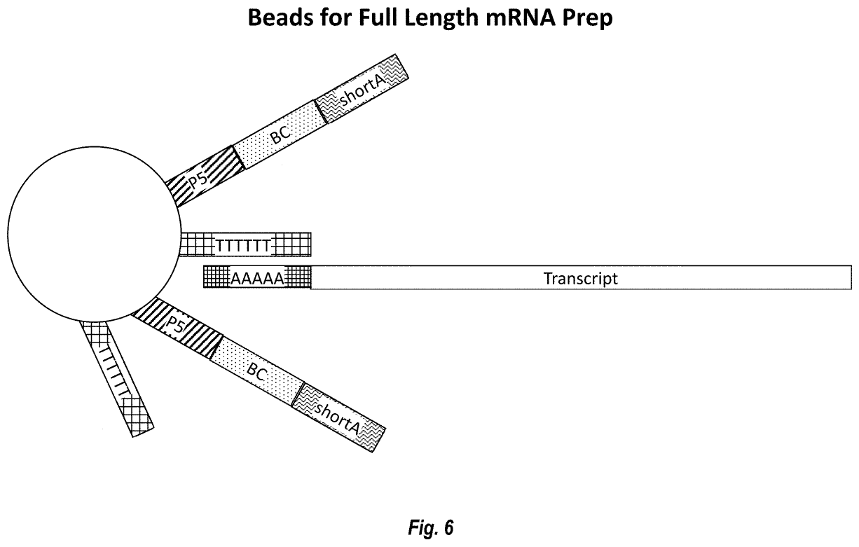

In some embodiments, the immobilized oligonucleotide than can capture transposomes from solution comprises an adapter sequence (such as P5 or P7, or their complements), a bead barcode, and a sequence for hybridizing to a sequence comprised in a second transposon of a transposome complex. As shown in the representative examples of FIG. 6-7, the immobilized oligonucleotide may comprise P5, a bead barcode (BC), and a sequence (short A) for hybridizing to a sequence (A′) comprised in a second transposon (wherein the second transposon comprises A′-ME′). The immobilized oligonucleotide may also comprise a sequencing adapter sequence (such as A14 or B15, or their complement). In this way, a user can capture mRNA and prepare DNA:RNA duplexes on a bead, and then hybridize transposomes to the bead to prepare cDNA fragments on the same bead. The use of activatable BLTs means that a user can prepare full- length DNA:RNA duplexes before preparing DNA fragments. In this way, fragmenting of partial DNA:RNA duplexes can be avoided, as the user can optimize conditions for DNA:RNA duplexes and not have concerns about fragmenting beginning before synthesis of a first strand of cDNA is complete.

In some embodiments, activatable RNA-BLTs comprise immobilized polyT sequences for capturing mRNA and immobilized oligonucleotides for capturing transposomes from solution. In some embodiments, such activatable BLTs can capture mRNA and allow preparation of DNA:RNA duplexes on the bead, after which transposomes can be hybridized to the immobilized oligonucleotides and allow for tagmentation. In some embodiments, only one type of transposome is hybridized to the immobilized oligonucleotides, thus allowing for symmetrical tagmentation on the BLT.

2. BLTs for Symmetric Tagmentation

In some embodiments, all transposome complexes comprise the same sequencing adapter sequence. In some embodiments, the first transposon comprised in all the transposome complexes is identical. In some embodiments, all the transposomes complexes immobilized on a bead (such as on a DNA-BLT or RNA-BLT) comprise the same first and second transposon. Use of such transposome complexes wherein the same adapter sequence can be added to both ends of a fragment may be termed “symmetrical tagmentation,” since these transposome complexes will lead to the same adapter being added to the 5′ end of both strands of a double-stranded fragment generated by tagmentation.

In some embodiments, fragmenting DNA:RNA duplexes with the transposome complexes is performed with transposome complexes comprising first transposons comprising the same adapter sequence. In some embodiments, all the transposome complexes are identical.

In some embodiments, BLTs for preparing RNA sequencing libraries comprise transposome complexes that comprise the same one or more adapter sequence. In some embodiments, the transposons in all transposome complexes comprised in a pool of BLTs are identical. In some embodiments, BLTs that comprise the same one or more adapter sequence means that both ends of a double-stranded cDNA fragment are tagged with the same adapter sequences. In some embodiments, both 5′ ends of double-stranded cDNA fragment of DNA:RNA duplex fragment incorporate the same one or more adapter. In some embodiments, both ends of the double-stranded cDNA comprise the same 5′ tag.

In some embodiments, methods using a symmetrical tagmentation step increases yield of sequencable fragments (i.e., each fragment having a different sequencing adapter sequence at each end of the fragment) as compared to standard asymmetrical tagmentation steps wherein more than one type of transposome complex is used for tagmentation. Asymmetrical tagmentation using 2 types of transposomes with different tags (A and B, such as a first-read sequencing adapter and a second-read sequencing adapter) causes loss of nearly half the reads from the amplified tagmentation products, because symmetrically and asymmetrically tagged products (A-A, B-B, A-B, B-A) are produced, but only the A-B and B-A are suitable for subsequent amplification and sequencing. In contrast, the present with BLTs for symmetrical tagmentation can increase the probability that resulting fragments will comprise both first-read and second-read sequencing adapters.

In some embodiments, methods using symmetrical tagmentation may increase yield of the library as compared to other library preparation methods.

A number of different methods for adding a second adapter after tagmentation are described herein. For example, a first-read sequencing adapter may be incorporated into double-stranded DNA or DNA:RNA duplex fragments during tagmentation, and a second-read sequencing adapter incorporated in a later step (such as by ligation). Exemplary methods will be described herein. In some embodiments, the present methods can improve library yield (compared to methods using asymmetrical tagmentation) by incorporating one sequencing adapter sequence through symmetrical tagmentation and another via use of a primer or oligonucleotide comprising a second sequencing adapter sequence.

3. BLTS for Asymmetric Tagmentation

In some embodiments, fragmenting the DNA:RNA duplexes with the transposome complexes is performed with two different transposome complexes, wherein the different transposome complexes comprise first transposons comprising different adapter sequences.

Use of two different transposome complexes wherein the different transposome complexes comprise first transposons comprising different adapter sequences may be termed “asymmetrical tagmentation,” since these transposome complexes can lead to different adapters being added to the 5′ end of the two strands of a double-stranded fragment generated by tagmentation. In some embodiments, asymmetrical tagmentation can allow for faster or easier workflows by incorporating two different adapters during the tagmentation step.

In some embodiments, at least some fragments are tagged with a first-read sequence adapter sequence at the 5′ end of one strand and with a second-read sequence adapter sequence at the 5′ end of the other strand using asymmetrical tagmentation.

4. BLTs Comprising Capture Oligonucleotides

In some embodiments, a solid support comprises transposomes and capture oligonucleotides. In some embodiments, the solid support is a bead. In some embodiments, beads comprise a capture oligonucleotide and a first polynucleotide that can be comprised in a transposome.

In some embodiments, the first polynucleotide further comprises a bead code. In some embodiments, the bead is comprised in a pool of beads, wherein each bead comprises an immobilized first polynucleotide comprising a different bead code as compared to the bead code comprised in other beads in the pool.

In some embodiments, BLTs further comprise capture oligonucleotides, wherein such beads can be used for capturing RNA, followed by preparing a first strand of cDNA to generate a DNA:RNA duplex immobilized and the bead, and finally preparing immobilized fragments from the DNA:RNA duplex. In some embodiments, the capture oligonucleotides are polyT sequences for capturing mRNA, and the beads allow for preparation of a library on the same bead used to capture the mRNA. In any of these embodiments, a second strand of cDNA may also be prepared after capture of the mRNA to prepare double-stranded cDNA.

In some embodiments, the BLTs comprising capture oligonucleotides are activatable BLTs comprising an immobilized oligonucleotide comprising a hybridization sequence that can be used to hybridize to transposome complexes.

FIGS. 11A-11C show preparation of a DNA:RNA duplex (FIG. 11A), hybridization of transposons to immobilized oligonucleotides comprising a hybridization sequence for generation of transposome complexes (FIG. 11B), and multiple tagmentation reactions mediated by the transposome complexes (FIG. 11C). After preparation of multiple fragments by tagmentation, a method as described herein (such as primer extension after symmetrical tagmentation) can be used to incorporate an additional adapter and prepare sequencable fragments.

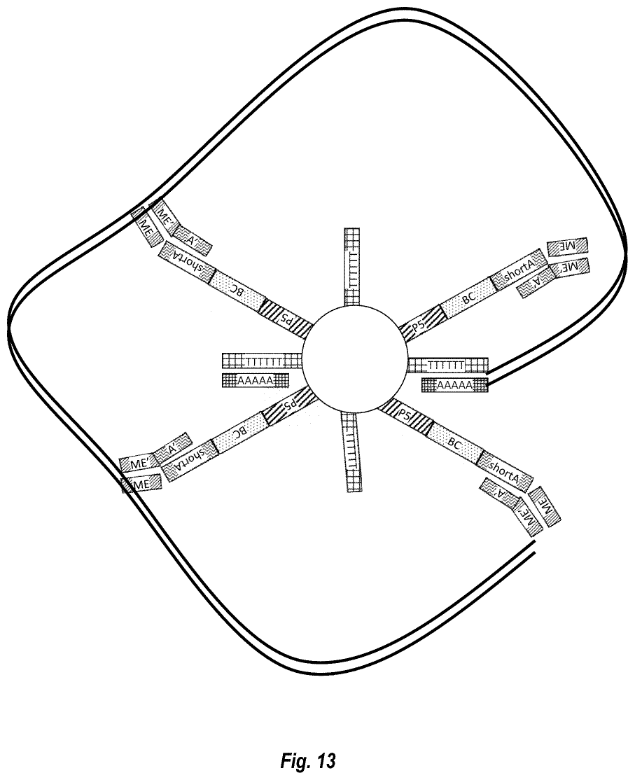

FIG. 12 shows a bead for full-length mRNA preparation with multiple capture oligonucleotides and multiple immobilized oligonucleotides that immobilize transposomes. FIG. 13 shows how multiple transposomes can allow for preparation of fragments from the full- length of a DNA:RNA duplex generated from an mRNA.

H. Delivery of Library Fragments to a Surface for Sequencing by BLTs

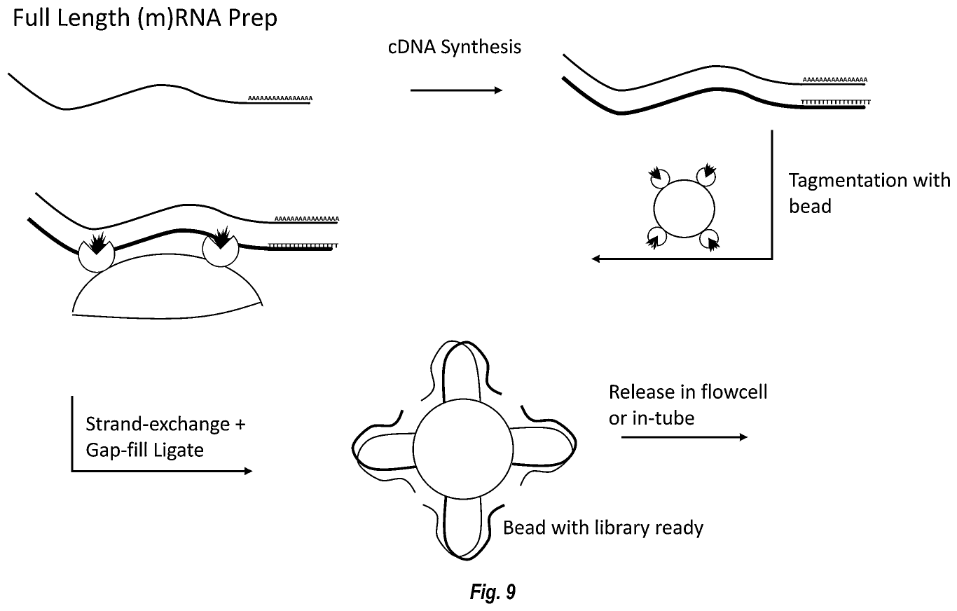

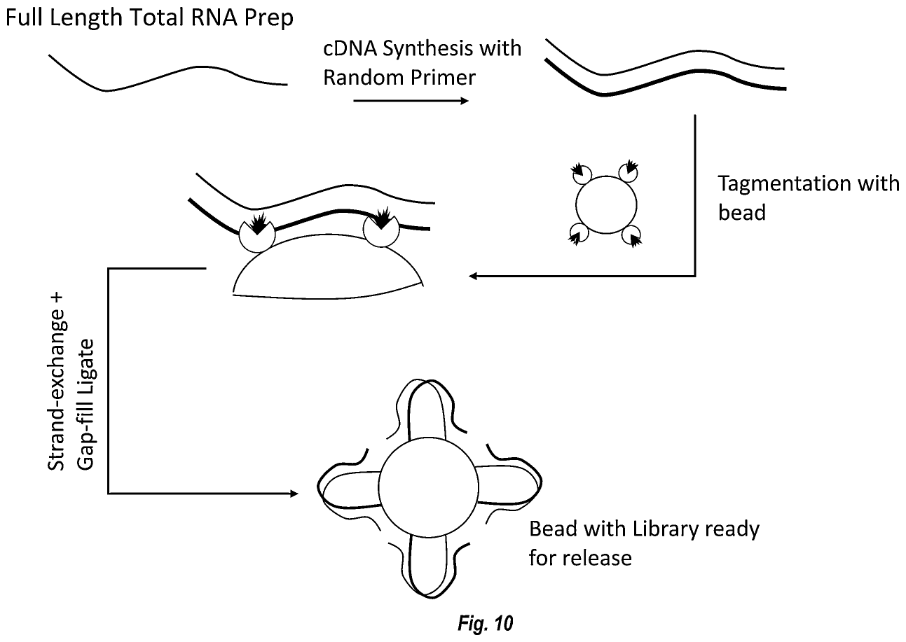

In some embodiments, a solid support with immobilized library fragments is used to deliver library fragments to a surface for sequencing. In some embodiments, the surface for sequencing is a flowcell. FIGS. 9 and 10 summarize how tagmentation and library preparation can be performed on a BLT, followed by release of fragments in a flowcell or tube. FIG. 9 uses a polyT primer to prepare a library from full-length mRNA, while FIG. 10 uses a random primer to prepare a library full-length total RNA.

In some embodiments, a library of fragments on a solid support is delivered to a surface for sequencing, while the fragments are immobilized on the solid support, and the fragments are then released and captured on the surface for sequencing. In some embodiments, a solid support with immobilized nucleic acids is delivered to a surface for sequencing, a library of immobilized fragments is generated on the solid support by tagmentation, and the fragments are then released and captured on the surface for sequencing.

In some embodiments, a method comprises, after the delivering, capturing the solid support with the immobilized library of DNA:RNA fragments on the surface for sequencing; releasing the immobilized fragments from the solid support; and capturing the fragments on the surface for sequencing. In some embodiments, a method further comprises sequencing the fragments on the surface for sequencing.

In some embodiments, BLTs can be used as solid-phase carriers. Exemplary uses of BLTs as solid-phase carriers are described in WO 2015/095226, which is incorporated herein in its entirety. In some embodiments, fragments released from a BLT can be recaptured on a region of the surface that is proximal to the site where the bead was captured on the surface for sequencing. In this way, all fragments from a given bead will be captured in close proximity on the surface for sequencing.

In some embodiments, a BLT with immobilized library fragments may be delivered to a surface for sequencing. In some embodiments, DNA or a DNA:RNA duplex on the surface of a BLT may have already been tagmented before the BLT is delivered to a surface for sequencing. For example, tagmentation on a BLT may occur in a reaction vessel, the BLTs are next delivered to a flowcell, and then fragments are released and captured on the flowcell.

In some embodiments, a BLT with immobilized nucleic acid may be delivered to a surface for sequencing. After this deliver, library fragments may be generated. For example, double-stranded DNA or DNA:RNA duplexes may be immobilized to the surface of a BLT, and tagmentation occurs after the BLT has been delivered to the surface for sequencing. For example, tagmentation on a BLT may occur with a flowcell, after which fragments are released and captured on the flowcell.

Release of fragments from a BLT may occur by a number of methods. For the example, fragments may be released by protease or SDS treatment. Alternatively, fragments can be produced by destruction of the bead, which releases fragments from a BLT. In any of these mechanisms for releasing fragments, fragments from a given bead can be released and captured on a surface for sequencing (such as a flowcell) in close proximity to aid in preparing a physical map of the sequence of an original DNA or RNA molecule.

In some embodiments, a BLT may comprise a hydrogel bead. In some embodiments, a hydrogel bead can be melted or dissolved to release fragments that were attached to the bead, such as methods disclosed in Publication Nos. WO 2019/028047 and WO 2019/028166, which are incorporated herein in their entirety.

In some embodiments, a BLT may comprise a degradable polyester bead. In some embodiments, a polyester bead can be degraded to release fragments that were attached to the bead, such as methods disclosed in Application No. WO PCT/US2021/040612, which is incorporated herein in its entirety. For example, each transposome complex may comprise a polynucleotide binding moiety that allows binding of a polynucleotide to another agent. In some embodiments, the polynucleotide binding moiety serves to bind a polynucleotide to a bead comprising a bead binding moiety. In some embodiments, each bead binding moiety is covalently bound to the polyester bead through a linker. In some embodiments, the linker comprises —N═CH—(CH2)3—CH═N—, —C(O)NH—(CH2)6—N═, or —C(O)NH—(CH2)6—N═CH—(CH2)3CH═N—.

In some embodiments, a degradable polyester bead is degraded by a temperature greater than 50° C., greater than 60° C., greater than 70° C., or greater than 80° C. In some embodiments, a degradable polyester bead is degraded by a temperature of 60° C. In some embodiments, a degradable polyester bead is degraded by an aqueous base. In some embodiments, the aqueous base is NaOH. In some embodiments, the NaOH is 1M-5M NaOH. In some embodiments, the NaOH is 3M NaOH (See Yeo et al., J Biomed Mater Res B Appl Biomater 87(2):562-9 (2008)). In some embodiments, a degradable polyester bead is degraded by aqueous NaOH at a temperature of from 50° C. to 90° C.

In some embodiments, a BLT with immobilized nucleic acid or fragments thereof can be allowed to contact a surface for sequencing by gravity settling. In some embodiments, a BLT with immobilized nucleic acid or fragments thereof can be attached to the surface for sequencing using receptors and ligands.

In some embodiments, proximity data can be used together with sequencing data on fragments to generate information on a given DNA or RNA that was comprised in a sample, as all fragments from a DNA or cDNA generated from an RNA would be generated on the same bead. The use of proximity data for preparing physical maps of immobilized polynucleotides can be performed using the methods described herein.

I. Primer Extension After Symmetrical Tagmentation on BLTs

In some embodiments, symmetrical tagmentation on BLTs leads to both ends of double-stranded DNA fragments having the same one or more adapter sequence.

In some embodiments, non-transferred ME′ sequences (from the second transposon) are melted off and gap-filled by PCR extension after tagmentation. In some embodiments, the ME′ sequences are removed by raising the temperature of the reaction.

In some embodiments, gap-filling is performed after non-transferred ME′ sequences are removed. In some embodiments, a primer is annealed to the gap-filled ME′ sequence. In some embodiments, this primer is used for extension and may be referred to as an extension primer. In some embodiments, the extension primer comprises a ME sequence. In some embodiments, the ME sequence comprised in the extension primer hybridizes to the gap-filled ME′ sequence.

In some embodiments, the extension primer also comprises a sequencing adapter sequence. In some embodiments, the sequence adapter sequence comprised in the extension primer was not comprised in the transposome complexes. In some embodiments, extension with the extension primer generates fragments comprising different sequencing adapter sequences at each end of the double-stranded fragments.

In some embodiments, a uracil-intolerant DNA is used for primer extension. In some embodiments, the second strand of cDNA is not extended because it comprises uracils, based on the strand-specific cDNA preparation described above.

In some embodiments, if the transposome complexes comprises an A14 sequence (or its complement), the extension primer comprises B15 (or its complement). In some embodiments, if the transposome complexes comprises an B15 sequence (or its complement), the extension primer comprises A14 (or its complement). In these representative examples, A14 and 15 only represent exemplary sequencing adapter sequences, and the present methods are not limited to such adapter sequences. Any set of paired adapter sequences of interest could be used in the transposome complexes and extension primers, and one skilled in the art would be well-aware of how sequencing is performed on different platforms and that such platforms may evolve over time.

1. Saturation of a DNA BLTs with Synthetic DNA

In some embodiments, after DNA BLTs (such as those that may be used as first solid supports in some methods) are used for performing tagmentation to generate fragments comprising a DNA-specific barcode, the DNA BLTs are bound with synthetic double-stranded DNA. In some embodiments, the DNA BLTs are saturated with synthetic double-stranded DNA. In some embodiments, the method comprises adding a synthetic double-stranded DNA to the first solid support after performing tagmentation on the first solid support.

In some embodiments, the DNA BLTs cannot bind (and tagment) nucleic acid after this saturating. Such saturation of DNA BLTs can allow for step-wise methods that next generate either cDNA or DNA:RNA duplexes from RNA for performing tagmentation on a second solid support. In some embodiments, the cDNA or DNA:RNA duplexes cannot be tagmented by the saturated DNA BLTs. Instead, the cDNA or DNA:RNA duplexes can be tagmented on a second solid support (i.e., RNA BLTs), allowing fragments generated from cDNA or DNA:RNA duplexes to be tagged with an RNA-specific barcode. In some embodiments, the synthetic double-stranded DNA comprises uracil, which blocks amplification of tagged fragments by certain high-fidelity DNA polymerases.

In some embodiments, saturation of DNA BLTs with synthetic double-stranded DNA can allow for methods that can be completed in a single reaction vessel. In some embodiments, saturation of DNA BLTs with synthetic double-stranded DNA obviates the need to partition DNA BLTs after tagmenting DNA from a sample.

J. Tags and DNA-specific and RNA-specific Barcodes

The terms “tag” and “tag domain” as used herein refer to a portion or domain of a polynucleotide that exhibits a sequence for a desired intended purpose or application. Some embodiments presented herein include a transposome complex comprising a polynucleotide having a 3′ portion comprising a transposon end sequence, and tag comprising a tag domain. Tag domains can comprise any sequence provided for any desired purpose. For example, in some embodiments, a tag domain comprises one or more restriction endonuclease recognition sites. In some embodiments, a tag domain comprises one or more regions suitable for hybridization with a primer for a cluster amplification reaction. In some embodiments, a tag domain comprises one or more regions suitable for hybridization with a primer for a sequencing reaction. It will be appreciated that any other suitable feature can be incorporated into a tag domain. In some embodiments, the tag domain comprises a sequence having a length from 5 bp to 200 bp. In some embodiments, the tag domain comprises a sequence having a length from 10 bp to 100 bp. In some embodiments, the tag domain comprises a sequence having a length from 20 bp to 50 bp. In some embodiments, the tag domain comprises a sequence having a length of 5, 6, 7, 8, 9, 10, 20, 30, 40, 50, 60, 70, 80, 90, 100, 150 or 200 bp.

The tag can include one or more functional sequences or components (e.g., primer sequences, anchor sequences, universal sequences, spacer regions, or index tag sequences) as needed or desired.

In some embodiments, at least 50%, 55%, 60%, 65%, 70%, 75%, 80%, 85%, 90%, 95%, 96%, 97%, 98%, or 99% of the first tags comprise the same tag domain. In some embodiments, the tag comprises a region for cluster amplification. In some embodiments, the tag comprises a region for priming a sequencing reaction.

In some embodiments, the method further comprises amplifying the fragments on the solid support by reacting a polymerase and an amplification primer corresponding to a portion of the first polynucleotide. In some embodiments, a portion of the first polynucleotide comprises an amplification primer. In some embodiments, the first tag of the first polynucleotide comprises an amplification primer.

In some embodiments, transposomes on an individual bead carry a unique index, and if a multitude of such indexed beads are employed, phased transcripts will result. In some embodiments for use with samples comprising RNA and DNA, RNA BLTs comprise a tag that is an index for identifying RNA BLTs (“iRNA”). In some embodiments for use with samples comprising RNA and DNA, DNA BLTs comprise a tag that is an index for identifying DNA BLTs (“iDNA”). An index for identifying RNA BLTs may be referred to as an “RNA-specific barcode” and an index for identifying DNA BLTs may be referred to as a “DNA-specific barcode.” Exemplary methods using DNA BLTs and RNA BLTs are shown in FIGS. 21-25.

In some embodiments, first polynucleotides comprise first tags. In some embodiments, second polynucleotides comprise second tags.

In some embodiments the first or second tag comprises an A14 primer sequence. In some embodiments, the first or second tag comprises a B15 primer sequence.

In some embodiments, a tag incorporated during tagmentation comprises an adapter sequence. In some embodiments, an adapter sequence may be added during tagmentation, during first strand cDNA synthesis, or via a primer after tagmentation.

In some embodiments, the adaptor sequence comprises a primer sequence, an index tag sequence, a capture sequence, a barcode sequence, a cleavage sequence, or a sequencing-related sequence, or a combination thereof. As used herein, a sequencing-related sequence may be any sequence related to a later sequencing step. A sequencing-related sequence may work to simplify downstream sequencing steps. For example, a sequencing-related sequence may be a sequence that would otherwise be incorporated via a step of ligating an adaptor to nucleic acid fragments. In some embodiments, the adaptor sequence comprises a P5 or P7 sequence (or their complement) to facilitate binding to a flowcell in certain sequencing methods. This disclosure is not limited to the type of adaptor sequences which could be used and a skilled artisan will recognize additional sequences which may be of use for library preparation and next generation sequencing.

In some embodiments, a first-read sequencing adapter sequence is incorporated during tagmentation and a second-read sequencing adapter sequence is incorporated via a primer after tagmentation. Different sequencing protocols use different “first-read sequencing adapters” and “second-read sequencing adapters,” and these adapters vary by manufacturer and equipment. In other words, the order and identity of sequencing reads is arbitrary for a given sequencing method. Thus, “first-read” and “second-read” sequencing adapters, as used herein, simply require the presence of two read sequencing adapters; they do not require that a specific adapter must be used for the first sequencing read versus second sequencing read in any downstream sequencing method after preparation of a sequencing library. Those skilled in the art could choose to first run a downstream sequencing reaction with a “second-read” sequencing adapter and then a “first-read” sequencing adapter if they so choose.

In some embodiments, the first-read and/or second-read sequencing adapter sequences comprise different primer binding sites.

K. Reverse Transcriptase Polymerase and cDNA Synthesis Reaction

In some embodiments, a reverse transcriptase polymerase is used for cDNA synthesis. As used herein, a “reverse transcriptase polymerase” refers to any RNA-dependent DNA polymerase that can catalyze DNA synthesis using RNA as a template. A reverse transcriptase polymerase can be used to synthesis a first strand of complementary DNA (cDNA) from RNA. In some embodiments, immobilized RNA molecules are converted to a DNA:RNA duplex via a reverse transcriptase polymerase.

In some embodiments, the reverse transcriptase polymerase only generates a single strand of cDNA. In some embodiments, the single strand of cDNA is bound to a target RNA, i.e., a reverse transcriptase polymerase is used to generate a DNA:RNA duplex from a target RNA.

In some embodiments, the reverse transcriptase polymerase generates two strands of cDNA, i.e., the reverse transcriptase generates double-stranded (ds) cDNA.

In some embodiments the reverse transcriptase polymerase is an M-MLV Reverse Transcriptase.

In some embodiments, the reagents for cDNA synthesis comprise a reverse transcriptase, random primers, oligo dT primers, dNTPs and/or an Rnase inhibitor. In some embodiments, both random primers and oligo dT primers are used in a cDNA synthesis reaction.

In some embodiments, reverse transcription is performed after RNA has been immobilized (i.e., captured) on a bead. In some embodiments, reverse transcription is performed in solution.

1. Preparation of cDNA Using Template Switching

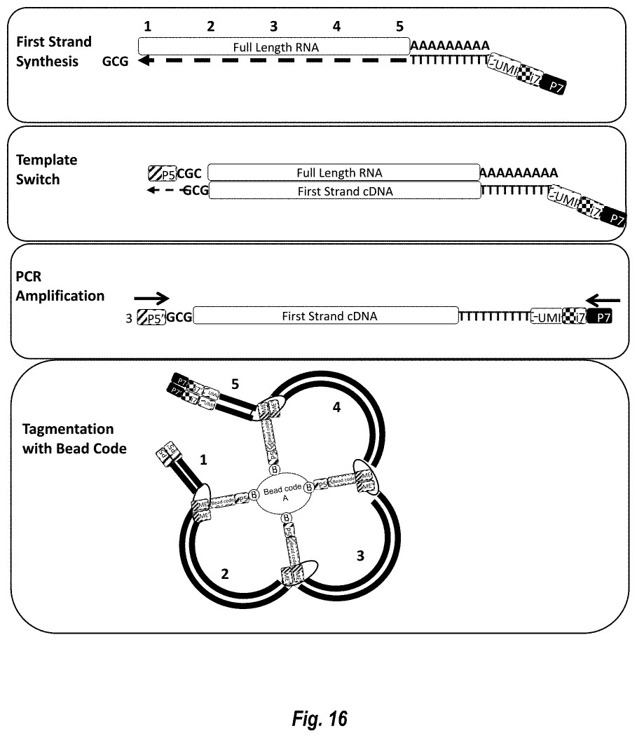

In some embodiments (such as FIG. 16), preparing double-stranded cDNA from the RNA is performed by template switching, as described in WO 2017/040306, WO 2015/168161, and WO 2010/117620, which are incorporated by reference in their entirety herein. In short, an oligo(dT) and/or randomer primer primes the first-strand cDNA synthesis reaction. When the reverse transcriptase (such as SMARTSCRIBE™) reaches the 5′ end of the mRNA, the enzyme's terminal transferase activity adds a few additional non-template nucleotides to the 3′ end of the cDNA. A template-switch oligonucleotide (TSO), designed to base-pair with this non-template nucleotide stretch, anneals and creates an extended template to enable the reverse transcriptase to continue replicating to the end of the oligonucleotide.

In some embodiments, a second strand of cDNA is synthesized using a template switching oligonucleotide primer (TSO primer). In some embodiments, the TSO primer further comprises a second amplification primer binding site. In some embodiments, the first strand synthesis primer is extended beyond the mRNA template and further copies the TSO primer strand. In some embodiments, the second strand of cDNA is synthesized using the TSO primer. In some embodiments, the second strand of cDNA is synthesized using the second amplification primer complimentary to the first strand of cDNA that is extended beyond the mRNA template to encompass the complimentary TSO strand.

An exemplary system employing a template switch oligonucleotide and compatible reverse transcriptase is SMRT-seq (Takara Bio).

2. Stranded cDNA Preparation

A variety of methods are known in the art that allow sequencing data to identify the mRNA strand that was the origin of a library fragment. Use of such “stranded” methods can allow the user to determine the sequence of the original mRNA strand using the sequence of the first strand of cDNA (without confounding data from a second strand of cDNA).

An exemplary method of stranded cDNA preparation is outlined in “TruSeq Stranded Total RNA Reference Guide,” Illumina, 2017. The mRNA is copied into a first strand of cDNA using reverse transcriptase in a First Strand Synthesis Actinomycin Mix, which allows RNA-dependent synthesis and prevents undesired DNA-dependent synthesis. The First Strand Synthesis Actinomycin Mix can improve strand specificity when generating a first strand of cDNA. Second strand cDNA synthesis is performed using DNA polymerase I and RNase H in a Second Strand Marking Mix, wherein dTTP has been replaced by dUTP. Incorporation of dUTP in the second strand of cDNA can quench amplification of this strand when a uracil-intolerant DNA polymerase is used (as described below in the amplification description).

In some embodiments, the nucleoside trisphosphates comprised in a composition for first strand cDNA synthesis comprises dCTP, dATP, dGTP, and dTTP.

In some embodiments, dTTP is replaced with dUTP in a second strand cDNA synthesis reaction for strand specificity. In some embodiments, a composition for second strand cDNA synthesis comprises dCTP, dATP, dGTP, and dUTP. In some embodiments, incorporation of dUTP in the second strand of cDNA suppresses amplification of the second strand of cDNA in the index PCR reaction during library preparation. In some embodiments, suppression of amplification of the second strand of cDNA allows for strand-specific methods.

In some embodiments, cDNA preparation is by a non-stranded method that does retain strand information from the mRNA.

L. Strand Exchange and Gap-Fill Ligation

After fragmenting of the DNA:RNA duplexes by the immobilized transposomes, a strand displacing polymerase (e.g., Bst polymerase) can be used to displace the RNA strand of the DNA:RNA fragments and to generate a second cDNA strand. In some embodiments, strand exchange is used to generate double-stranded DNA fragments. In some embodiments, the strand exchange steps remove the RNA strand of the fragments from the DNA:RNA duplex and replaces the RNA with a second strand of DNA. In some embodiments, strand exchange via a strand displacing polymerase converts fragments comprising DNA:RNA in double-stranded DNA fragments.

In some embodiments, gaps in the DNA sequence left after the transposition event can also be filled in using a strand displacement extension reaction, such one comprising a Bst DNA polymerase and dNTP mix. In some embodiments, a gap-fill ligation is performed using an extension-ligation mix buffer.

The library of double-stranded DNA fragments can then optionally be amplified (such as with cluster amplification) and sequenced with a sequencing primer.

M. Amplification

The present disclosure further relates to amplification of the immobilized DNA fragments produced according to the methods provided herein. The immobilized DNA fragments produced by surface bound transposome mediated tagmentation can be amplified according to any suitable amplification methodology known in the art. In some embodiments, the immobilized DNA fragments are amplified on a solid support. In some embodiments, the solid support is the same solid support upon which the surface bound tagmentation occurs. In such embodiments, the methods and compositions provided herein allow sample preparation to proceed on the same solid support from the initial sample introduction step through amplification and optionally through a sequencing step.

For example, in some embodiments, the immobilized DNA fragments are amplified using cluster amplification methodologies as exemplified by the disclosures of U.S. Pat. Nos. 7,985,565 and 7,115,400, the contents of each of which is incorporated herein by reference in its entirety. The incorporated materials of U.S. Pat. Nos. 7,985,565 and 7,115,400 describe methods of solid-phase nucleic acid amplification which allow amplification products to be immobilized on a solid support in order to form arrays comprised of clusters or “colonies” of immobilized nucleic acid molecules. Each cluster or colony on such an array is formed from a plurality of identical immobilized polynucleotide strands and a plurality of identical immobilized complementary polynucleotide strands. The arrays so-formed are generally referred to herein as “clustered arrays”. The products of solid-phase amplification reactions such as those described in U.S. Pat. Nos. 7,985,565 and 7,115,400 are so-called “bridged” structures formed by annealing of pairs of immobilized polynucleotide strands and immobilized complementary strands, both strands being immobilized on the solid support at the 5′ end, in some embodiments via a covalent attachment. Cluster amplification methodologies are examples of methods wherein an immobilized nucleic acid template is used to produce immobilized amplicons. Other suitable methodologies can also be used to produce immobilized amplicons from immobilized DNA fragments produced according to the methods provided herein. For example, one or more clusters or colonies can be formed via solid-phase PCR whether one or both primers of each pair of amplification primers are immobilized.

In other embodiments, the immobilized DNA fragments are amplified in solution. For example, in some embodiments, the immobilized DNA fragments are cleaved or otherwise liberated from the solid support and amplification primers are then hybridized in solution to the liberated molecules. In other embodiments, amplification primers are hybridized to the immobilized DNA fragments for one or more initial amplification steps, followed by subsequent amplification steps in solution. Thus, in some embodiments an immobilized nucleic acid template can be used to produce solution-phase amplicons.

It will be appreciated that any of the amplification methodologies described herein or generally known in the art can be utilized with universal or target-specific primers to amplify immobilized DNA fragments. Suitable methods for amplification include, but are not limited to, the polymerase chain reaction (PCR), strand displacement amplification (SDA), transcription mediated amplification (TMA) and nucleic acid sequence-based amplification (NASBA), as described in U.S. Pat. No. 8,003,354, which is incorporated herein by reference in its entirety. The above amplification methods can be employed to amplify one or more nucleic acids of interest. For example, PCR, including multiplex PCR, SDA, TMA, NASBA and the like can be utilized to amplify immobilized DNA fragments. In some embodiments, primers directed specifically to the nucleic acid of interest are included in the amplification reaction.

Other suitable methods for amplification of nucleic acids can include oligonucleotide extension and ligation, rolling circle amplification (RCA) (Lizardi et al., Nat. Genet. 19:225-232 (1998), which is incorporated herein by reference) and oligonucleotide ligation assay (OLA) (See generally U.S. Pat. Nos. 7,582,420, 5,185,243, 5,679,524 and 5,573,907; EP 0 320 308 B1; EP 0 336 731 B1; EP 0 439 182 B1; WO 90/01069; WO 89/12696; and WO 89/09835, all of which are incorporated by reference) technologies. It will be appreciated that these amplification methodologies can be designed to amplify immobilized DNA fragments. For example, in some embodiments, the amplification method can include ligation probe amplification or oligonucleotide ligation assay (OLA) reactions that contain primers directed specifically to the nucleic acid of interest. In some embodiments, the amplification method can include a primer extension-ligation reaction that contains primers directed specifically to the nucleic acid of interest. As a non-limiting example of primer extension and ligation primers that can be specifically designed to amplify a nucleic acid of interest, the amplification can include primers used for the GoldenGate assay (Illumina, Inc., San Diego, Calif.) as exemplified by U.S. Pat. No. 7,582,420 and 7,611,869, each of which is incorporated herein by reference in its entirety.

Exemplary isothermal amplification methods that can be used in a method of the present disclosure include, but are not limited to, Multiple Displacement Amplification (MDA) as exemplified by, for example Dean et al., Proc. Natl. Acad. Sci. USA 99:5261-66 (2002) or isothermal strand displacement nucleic acid amplification exemplified by, for example U.S. Pat. No. 6,214,587, each of which is incorporated herein by reference in its entirety. Other non-PCR-based methods that can be used in the present disclosure include, for example, strand displacement amplification (SDA) which is described in, for example Walker et al., Molecular Methods for Virus Detection, Academic Press, Inc., 1995; U.S. Pat. Nos. 5,455,166, and 5,130,238, and Walker et al., Nucl. Acids Res. 20:1691-96 (1992) or hyperbranched strand displacement amplification which is described in, for example Lage et al., Genome Research 13:294-307 (2003), each of which is incorporated herein by reference in its entirety. Isothermal amplification methods can be used with the strand-displacing Phi 29 polymerase or Bst DNA polymerase large fragment, 5′−>3′ exo—for random primer amplification of genomic DNA. The use of these polymerases takes advantage of their high processivity and strand displacing activity. High processivity allows the polymerases to produce fragments that are 10-20 kb in length. As set forth above, smaller fragments can be produced under isothermal conditions using polymerases having low processivity and strand-displacing activity such as Klenow polymerase. Additional description of amplification reactions, conditions and components are set forth in detail in the disclosure of U.S. Pat. No. 7,670,810, which is incorporated herein by reference in its entirety.

Another nucleic acid amplification method that is useful in the present disclosure is Tagged PCR which uses a population of two-domain primers having a constant 5′ region followed by a random 3′ region as described, for example, in Grothues et al. Nucleic Acids Res. 21(5):1321-2 (1993), incorporated herein by reference in its entirety. The first rounds of amplification are carried out to allow a multitude of initiations on heat denatured DNA based on individual hybridization from the randomly synthesized 3′ region. Due to the nature of the 3′ region, the sites of initiation are contemplated to be random throughout the genome. Thereafter, the unbound primers can be removed and further replication can take place using primers complementary to the constant 5′ region.

1. Amplification with a Uracil-Intolerant DNA Polymerase

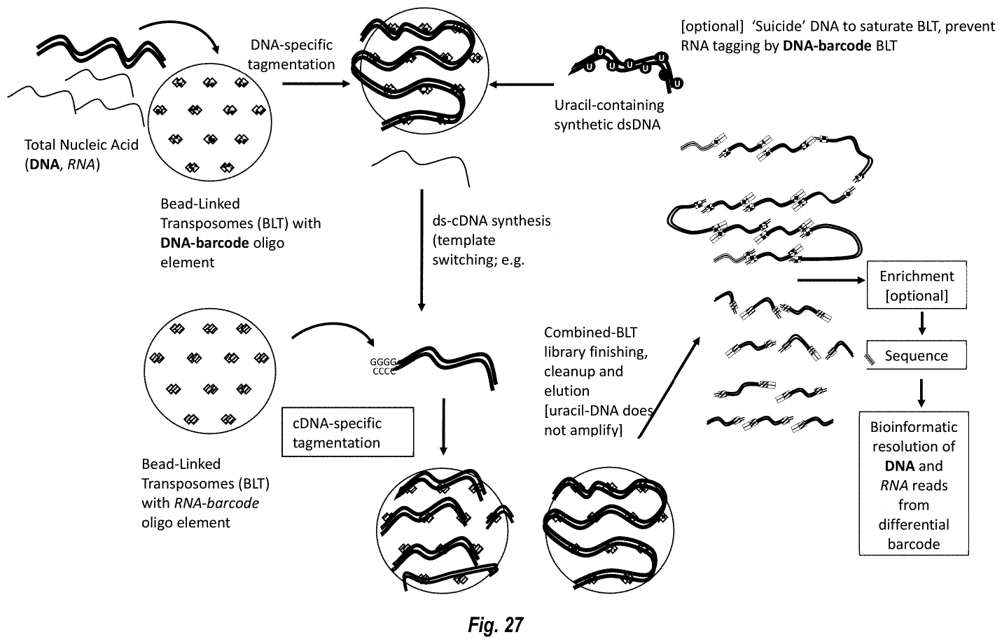

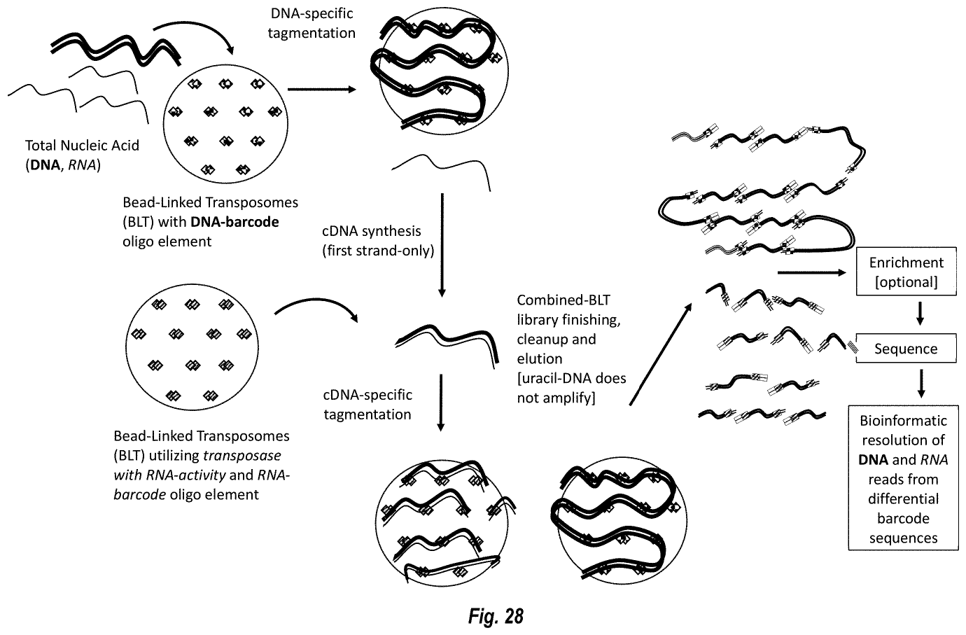

In some embodiments, after performing tagmentation on DNA on a first solid support (such as DNA bead-linked transposomes (BLTs)) to incorporate a DNA-specific barcode into the DNA fragments, synthetic double-stranded DNA is added to the first solid support. In some embodiments, the synthetic double-stranded DNA binds to any transposome complexes on the first solid support that are not already bound by DNA fragments. In some embodiments, the synthetic double-stranded DNA is “suicide DNA” that cannot be later amplified. In some embodiments, the synthetic double-stranded DNA comprises uracil and a uracil-intolerant DNA polymerase is used for amplification in later steps (see, for example, the methods outlined in FIGS. 27 and 28).

In some embodiments, the uracil-intolerant DNA polymerase is a high-fidelity or proofreading DNA polymerase. One skilled in the art would be aware that many proofreading DNA polymerase are unable to amplify uracil-containing templates due to a “uracil-binding pocket” that detects uracil residues in the template strand and stalls further DNA synthesis (See “Thermo Scientific Phusion DNA Polymerases,” Thermo Fisher 2015). In some embodiments, the high-fidelity DNA polymerase is KAPA HiFi HotStart (Roche) or Phusion (Thermo Fisher). Uses of uracil-intolerant polymerases are also described in Application No. PCT/US2021/036599 and Mulqueen et al., High-content single-cell combinatorial indexing, bioRxiv preprint available at doi.org/10.1101/2021.01.11.425995 posted Jan. 12, 2021, each of which is incorporated by reference herein in its entirety. In some embodiments, a transposome complex comprises a uracil base immediately following a mosaic end sequence, and use of a uracil-intolerant polymerase prevents extension beyond the mosaic end, as described in Mulqueen et al.

Similarly, a uracil-intolerant DNA polymerase may be used in stranded methods of cDNA preparation. In some embodiments, the presence of uracil in a second strand of cDNA prepared from RNA in a sample can quench amplification of this second strand when a uracil-intolerant DNA polymerase is used. In this way, the amplified cDNA is limited to that generated from the first strand of cDNA and allows identification of the mRNA strand that was comprised in the sample.

2. Selective Amplification

In some embodiments, the DNA-specific barcode and the RNA-specific barcode allow for selective amplification. “Selective amplification” refers to amplification of fragments that originated from DNA in a sample (i.e., fragments tagged with a DNA-specific barcode) or amplification of fragments that originated from RNA in a sample (i.e., fragments tagged with an RNA-specific barcode). The selective amplification allows the user to amplify (and potentially sequence) only fragments originating from DNA or fragments originating from RNA. Alternatively, the user may choose to amplify both the fragments originating from DNA and RNA.

Depending on the user's goal for the experiment, only the DNA or RNA from a given sample may be of interest. Alternatively, the user may desire a multi-omic analysis of a sample to assess both DNA and RNA.

In some embodiments, the DNA-specific barcode and the RNA-specific barcode comprise different primer binding sequences. In some embodiments, the method further comprises amplifying tagged fragments comprising the DNA-specific barcode using a primer that binds the primer binding sequence comprised in the DNA-specific barcode. In some embodiments, the method further comprises amplifying tagged fragments comprising the RNA-specific barcode using a primer that binds the primer binding sequence comprised in the RNA-specific barcode. In some embodiments, the method further comprises amplifying tagged fragments comprising the DNA-specific barcode and tagged fragments comprising the RNA-specific barcode using a primer mix comprising a primer that binds the primer binding sequence comprised in the DNA-specific barcode and a primer that binds the primer binding sequence comprised in the RNA-specific barcode.

N. Sequencing

The present disclosure further relates to sequencing of the immobilized DNA fragments produced according to the methods provided herein. In some embodiments, the method comprises sequencing tagged fragments or amplified tagged fragments. The immobilized DNA fragments may be those generated from dsDNA comprised in a sample, ds-cDNA generated from RNA comprised in a sample, or ds-DNA generated from strand exchange after tagmenting of DNA:RNA duplexes generated from RNA comprised in a sample. In some embodiments, the immobilized DNA fragments may comprise DNA-specific barcodes or RNA-specific barcodes, such that bioinformatic resolution after sequencing can differentiate those fragments that originated from DNA of a sample versus those fragments that originated from RNA of a sample.

In some embodiments, the fragments of a DNA:RNA duplexes are sequenced directly without strand exchange.

The immobilized DNA fragments produced by surface bound transposome mediated tagmentation can be sequenced according to any suitable sequencing methodology, such as direct sequencing, including sequencing by synthesis, sequencing by ligation, sequencing by hybridization, nanopore sequencing and the like. In some embodiments, the immobilized DNA fragments are sequenced on a solid support. In some embodiments, the solid support for sequencing is the same solid support upon which the surface bound tagmentation occurs. In some embodiments, the solid support for sequencing is the same solid support upon which the amplification occurs.

One exemplary sequencing methodology is sequencing-by-synthesis (SBS). In SBS, extension of a nucleic acid primer along a nucleic acid template (e.g. a target nucleic acid or amplicon thereof) is monitored to determine the sequence of nucleotides in the template. The underlying chemical process can be polymerization (e.g. as catalyzed by a polymerase enzyme). In a particular polymerase-based SBS embodiment, fluorescently labeled nucleotides are added to a primer (thereby extending the primer) in a template dependent fashion such that detection of the order and type of nucleotides added to the primer can be used to determine the sequence of the template.

Flowcells provide a convenient solid support for housing amplified DNA fragments produced by the methods of the present disclosure. One or more amplified DNA fragments in such a format can be subjected to an SBS or other detection technique that involves repeated delivery of reagents in cycles. For example, to initiate a first SBS cycle, one or more labeled nucleotides, DNA polymerase, etc., can be flowed into/through a flowcell that houses one or more amplified nucleic acid molecules. Those sites where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flowcell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with amplicons produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568 and U.S. Pat. No. 6,274,320, each of which is incorporated herein by reference). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence- based detection systems are not necessary for pyrosequencing procedures. Useful fluidic systems, detectors and procedures that can be adapted for application of pyrosequencing to amplicons produced according to the present disclosure are described, for example, in WIPO Pat. App. Ser. No. PCT/US11/57111, US 2005/0191698 A1, U.S. Pat. No. 7,595,883, and U.S. Pat. No. 7,244,559, each of which is incorporated herein by reference.

Some embodiments can utilize methods involving the real-time monitoring of DNA polymerase activity. For example, nucleotide incorporations can be detected through fluorescence resonance energy transfer (FRET) interactions between a fluorophore-bearing polymerase and y-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008), the disclosures of which are incorporated herein by reference.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

Another useful sequencing technique is nanopore sequencing (see, for example, Deamer et al. Trends Biotechnol. 18, 147-151 (2000); Deamer et al. Acc. Chem. Res. 35:817-825 (2002); Li et al. Nat. Mater. 2:611-615 (2003), the disclosures of which are incorporated herein by reference). In some nanopore embodiments, the target nucleic acid or individual nucleotides removed from a target nucleic acid pass through a nanopore. As the nucleic acid or nucleotide passes through the nanopore, each nucleotide type can be identified by measuring fluctuations in the electrical conductance of the pore. (U.S. Pat. No. 7,001,792; Soni et al. Clin. Chem. 53, 1996-2001 (2007); Healy, Nanomed. 2, 459-481 (2007); Cockroft et al. J. Am. Chem. Soc. 130, 818-820 (2008), the disclosures of which are incorporated herein by reference).

Exemplary methods for array-based expression and genotyping analysis that can be applied to detection according to the present disclosure are described in US Pat. Nos. 7,582,420; 6,890,741; 6,913,884 or 6,355,431 or US Pat. Pub. Nos. 2005/0053980 A1; 2009/0186349 A1 or US 2005/0181440 A1, each of which is incorporated herein by reference.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flowcell can be configured and/or used in an integrated system for detection of target nucleic acids. Exemplary flowcells are described, for example, in US 2010/0111768 A1 and U.S. Ser. No. 13/273,666, each of which is incorporated herein by reference. As exemplified for flowcells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeg™ platform (Illumina, Inc., San Diego, Calif.) and devices described in U.S. Ser. No. 13/273,666, which is incorporated herein by reference.

O. Physical Maps of Immobilized Polynucleotide Molecules

Also presented herein are methods of generating a physical map of immobilized polynucleotides. The methods can advantageously be exploited to identify clusters likely to contain linked sequences (i.e., the first and second portions from the same target polynucleotide molecule). The relative proximity of any two clusters resulting from an immobilized polynucleotide thus provides information useful for alignment of sequence information obtained from the two clusters. Specifically, the distance between any two given clusters on a solid surface is positively correlated with the probability that the two clusters are from the same target polynucleotide molecule, as described in greater detail in WO 2012/025250, which is incorporated herein by reference in its entirety.

As an example, in some embodiments, long DNA:RNA duplex molecules stretching out over the surface of a flowcell are tagmented in situ, resulting in a line of connected DNA:RNA bridges across the surface of the flowcell. Further, a physical map of the immobilized DNA:RNA can then be generated before or after strand exchange generates immobilized DNA. The physical map thus correlates the physical relationship of clusters after immobilized DNA is amplified. Specifically, the physical map is used to calculate the probability that sequence data obtained from any two clusters are linked, as described in the incorporated materials of WO 2012/025250.

In some embodiments, the physical map is generated by imaging the DNA to establish the location of the immobilized DNA molecules across a solid surface. In some embodiments, the immobilized DNA is imaged by adding an imaging agent to the solid support and detecting a signal from the imaging agent. In some embodiments, the imaging agent is a detectable label. Suitable detectable labels include, but are not limited to, protons, haptens, radionuclides, enzymes, fluorescent labels, chemiluminescent labels, and/or chromogenic agents. For example, in some embodiments, the imaging agent is an intercalating dye or non-intercalating DNA binding agent. Any suitable intercalating dye or non-intercalating DNA binding agent as are known in the art can be used, including, but not limited to those set forth in U.S. 2012/0282617, which is incorporated herein by reference in its entirety.

In some embodiments, the immobilized DNA:RNA duplexes are further fragmented to liberate a free end prior to strand exchange and cluster generation. Cleaving bridged structures can be performed using any suitable methodology as is known in the art, as exemplified by the incorporated materials of WO 2012/025250. For example, cleavage can occur by incorporation of a modified nucleotide, such as uracil as described in WO 2012/025250, by incorporation of a restriction endonuclease site, or by applying solution-phase transposome complexes to the bridged DNA structures, as described elsewhere herein.

In certain embodiments, a plurality of RNA is flowed onto a flowcell comprising a plurality of nano-channels, the nano-channel having a plurality of transposome complexes immobilized thereto. As used herein, the term nano-channel refers to a narrow channel into which a long linear nucleic acid molecule is flowed. In some embodiments, no more than 1, 2, 3, 4, 5, 6, 7, 8, 9, 10, 11, 12, 13, 14, 15, 16, 17, 18, 19, 20, 30, 40, 50, 60 70, 80, 90, 100, 200, 300, 400, 500, 600, 700, 800, 900 or no more than 1000 individual long strands of target RNA are flowed into each nano-channel. In some embodiments the individual nano-channels are separated by a physical barrier which prevents individual long strands of target RNA from interacting with multiple nano-channels. In some embodiments, the solid support comprises at least 10, 50, 100, 200, 500, 1000, 3000, 5000, 10000, 30000, 50000, 80000 or 100000 nano-channels. In some embodiments, transposomes bound to the surface of a nano-channel tagment the RNA. Contiguity mapping can then be performed, for example, by following the clusters down the length of one of these channels. In some embodiments, the long strand of target RNA can be at least 0.1kb, 1kb, 2kb, 3kb, 4kb, 5kb, 6kb, 7kb, 8kb, 9kb, 10kb, 15kb, 20kb, 25kb, 30kb, 35kb, 40kb, 45kb, 50kb, 55kb, 60kb, 65kb, 70kb, 75kb, 80kb, 85kb, 90kb, 95kb, 100kb, 150kb, 200kb, 250kb, 300kb, 350kb, 400kb, 450kb, 500kb, 550kb, 600kb, 650kb, 700kb, 750kb, 800kb, 850kb, 900kb, 950kb, 1000kb, 5000kb, 10000kb, 20000kb, 30000kb, or 50000kb in length. In some embodiments, the long strand of target RNA is no more than 0.1kb, 1kb, 2kb, 3kb, 4kb, 5kb, 6kb, 7kb, 8kb, 9kb, 10kb, 15kb, 20kb, 25kb, 30kb, 35kb, 40kb, 45kb, 50kb, 55kb, 60kb, 65kb, 70kb, 75kb, 80kb, 85kb, 90kb, 95kb, 100kb, 150kb, 200kb, 250kb, 300kb, 350kb, 400kb, 450kb, 500kb, 550kb, 600kb, 650kb, 700kb, 750kb, 800kb, 850kb, 900kb, 950kb, or no more than 1000kb in length. As an example, a flowcell having 1000 or more nano-channels with mapped immobilized tagmentation products in the nano-channels can be used to sequence the genome of an organism with short ‘positioned’ reads. In some embodiments, mapped immobilized tagmentation products in the nano-channels can be used resolve haplotypes. In some embodiments, mapped immobilized tagmentation products in the nano-channels can be used to resolve phasing issues.

II. Methods of Preparing RNA Sequencing Libraries Using Bead-Linked Transposomes and cDNA Synthesis in Solution

In some embodiments, cDNA is synthesized in solution from a sample comprising RNA as a first step of a library preparation. In other words, a DNA:RNA duplex may be generated in solution before tagmentation by a BLT (as shown in FIG. 2). In some embodiments, the DNA:RNA duplex is then captured on a BLT by a capture oligonucleotide. In some embodiments, the DNA:RNA duplexes bind directly to BLTs based on affinity for transposases comprised in transposome complexes.

In some embodiments, cDNA synthesis is performed by a reverse transcriptase. In some embodiments, this cDNA synthesis yield DNA:RNA duplexes, wherein a strand of DNA is generated that can hybridize to a strand of RNA. In some embodiments, a reverse transcriptase polymerase is added to a sample comprising RNA under conditions to synthesize cDNA. In some embodiments, conditions to synthesize cDNA include the presence of nucleotides and/or primers that can bind to RNA (such as polyT primers and/or randomer primers). In some embodiments, the reaction mixture for preparing DNA:RNA duplexes comprises an oligo dT primer, a reverse transcriptase, and nucleotides. In some embodiments, the DNA:RNA duplexes synthesis a first strand of cDNA at a reaction temperature of 42° C.

In some embodiments, the reverse transcriptase only prepares DNA from the RNA (without generating additional copies of the DNA to yield double-stranded DNA).

In some embodiments, cDNA preparation is done in solution to generate DNA:RNA duplexes or to generate double-stranded cDNA.

In some embodiments, DNA:RNA duplexes generated in solution can then be bound to BLTs and tagmented. After stopping or removing the transposases, strand exchange can be performed followed by gap-filling and ligation, and the library can then be released. In some embodiments, strand exchange is not required if double-stranded cDNA is prepared in solution and then tagmented. In some embodiments, these methods can be performed in-tube or in-flowcell.

In some embodiments, a method of preparing an immobilized library of tagged DNA:RNA fragments from target RNA comprises adding a reverse transcriptase polymerase to a sample comprising target RNA under conditions to synthesize cDNA and generate DNA:RNA duplexes; immobilizing DNA:RNA duplexes to a solid support having transposome complexes immobilized thereon, wherein the transposome complexes comprise a transposase bound to a first polynucleotide comprising a 3′ portion comprising a transposon end sequence, and a first tag; wherein the sample is applied to the solid support under conditions wherein the DNA:RNA duplexes bind to capture oligonucleotides or transposases directly; and fragmenting the DNA:RNA duplexes with the transposome complexes under conditions wherein the DNA:RNA duplexes are tagged on the 5′ end of one strand, thereby producing an immobilized library of DNA:RNA fragments wherein at least one strand is 5′-tagged with the first tag. In some embodiments, the 5′ end of one strand is the 5′ end of the RNA strand. In some embodiments, the 5′ end of one strand is the 5′ end of the DNA strand.

In some embodiments, a method of preparing an immobilized library of tagged DNA:RNA fragments from target RNA comprises applying a sample comprising target RNA to a solid support having capture oligonucleotides immobilized thereon; adding a reverse transcriptase polymerase under conditions to synthesize cDNA and generate immobilized DNA:RNA duplexes on the capture oligonucleotides; and fragmenting the DNA:RNA duplexes with the transposome complexes in solution under conditions wherein the DNA:RNA duplexes are tagged on the 5′ end of one strand, thereby producing an immobilized library of DNA:RNA fragments wherein at least one strand is 5′-tagged with the first tag. In some embodiments, the RNA is mRNA, and the capture oligonucleotide comprises a polyT sequence. In some embodiments, the library of fragments comprises DNA:RNA fragments generated from the 3′ end of one or more RNA. In some embodiments, the capture oligonucleotide further comprises a first-read sequencing adapter sequence, bead code, and/or one or more additional adapter sequences. In some embodiments, the transposomes complexes in solution comprise a first transposome comprising a second-read sequence adapter sequence and/or one or more additional adapter sequences. In some embodiments, the library of DNA:RNA fragments are sequenced without amplifying fragments before sequencing.

III. Methods of Preparing RNA Sequencing Libraries with 3′ UMIs

Current methods of UMI sequencing alone enable quantitative sequencing of only a fragment of the transcript, which hinders high resolution isoform identification. In some embodiments, coupling linked-long read technology with 3′ UMI attachment described herein enables quantitative sequencing of RNA libraries, as well as isoform identification by linking all fragments of the original transcript together with the 3′ UMI. In some embodiments, bead-linked transposomes (BLTs) with a common bead code identifier can generate linked long reads for full- length RNA. While the present methods with UMIs could have many uses, they may be particularly useful for alternative splicing studies, wherein identification of sequences that originated from different RNA molecules is critical.

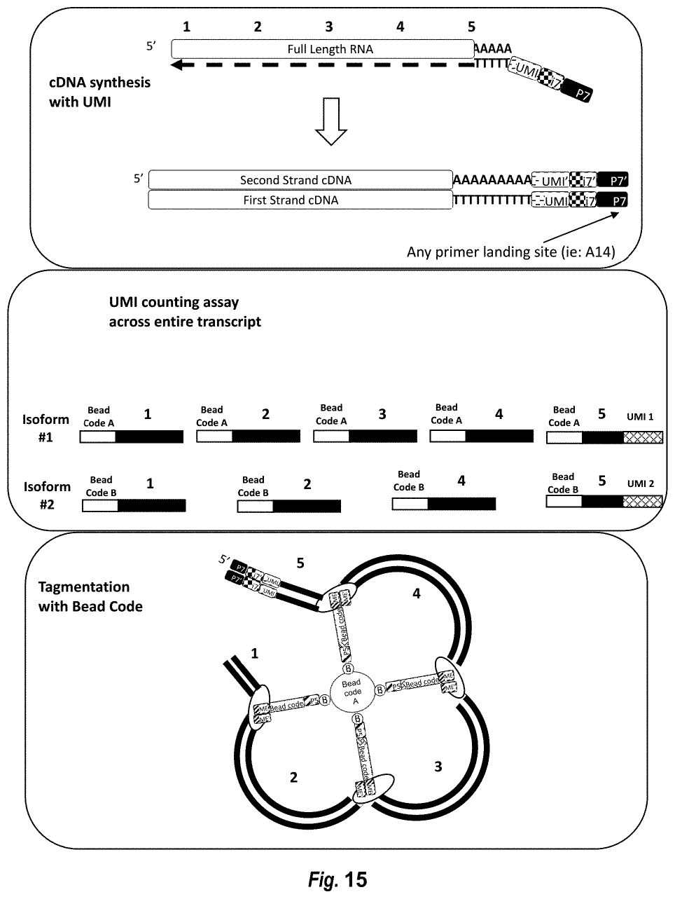

In some embodiments, the method combines 3′ UMI tagging for accurate quantification and linked long read bead codes, as exemplified by FIGS. 15 and 16. In some embodiments, the 3′ UMI tagging is performed before amplification. In some embodiments, incorporation of the 3′ UMI preamplification of cDNA and before tagmentation makes the method compatible with single cells and ultra-low RNA inputs. In some embodiments, the methods enable a full-length RNA counting assay for single cells. In some embodiments, the method comprises template switching.

In some embodiments, a method of preparing a library of double-stranded DNA fragments from RNA comprises preparing a first strand of cDNA from a full-length RNA in a sample using a polyT primer comprising a UMI and a first-read sequencing adapter sequence; preparing a second strand of cDNA to generate double-stranded cDNA; applying the double- stranded cDNA to a bead having transposome complexes immobilized thereon, wherein each transposome complex comprises a transposase; a first transposon comprising a 3′ transposon end sequence; and a second transposon comprising a sequence all or partially complementary to the transposon end sequence and a hybridization sequence; wherein the transposome complex is immobilized by binding of the hybridization sequence to an oligonucleotide immobilized to a bead, wherein said oligonucleotide comprises a 5′ affinity element, a first-read sequencing adapter sequence, a bead code, and a sequence all or partially complementary to the hybridization sequence; immobilizing the double-stranded cDNA and performing tagmentation on the bead to prepare double-stranded DNA fragments; removing the second transposon; hybridizing a primer comprising a second-read sequencing adapter sequence and a sequence all or partially complementary to the transposon end sequence to the transposon end sequence; and performing gap-filling and extension to prepare double-stranded DNA fragments comprising the first-read sequencing adapter and the second-read sequencing adapter.

In some embodiments, a method of preparing a library of double-stranded DNA fragments from RNA comprises preparing a first strand of cDNA from a full-length RNA in a sample using a polyT primer comprising a UMI and a first-read sequencing adapter sequence; preparing a second strand of cDNA to generate double-stranded cDNA; applying the double-stranded cDNA to a bead having transposome complexes immobilized thereon, wherein each transposome complex comprises a transposase; a first transposon comprising a 3′ transposon end sequence, a bead code, and a second-read sequencing adapter sequence; wherein the first transposon further comprises a 5′ affinity element for immobilizing the transposome complex to the solid support; and a second transposon comprising a sequence all or partially complementary to the transposon end sequence; immobilizing the double-stranded cDNA and performing tagmentation on the bead to prepare double-stranded DNA fragments; removing the second transposon; hybridizing a primer comprising a second-read sequencing adapter sequence and a sequence all or partially complementary to the transposon end sequence to the transposon end sequence; and performing gap- filling and extension to prepare double-stranded DNA fragments comprising the first-read sequencing adapter and the second-read sequencing adapter.

In some embodiments, the primer comprises a 5′ portion comprising the second-read sequence adapter and a 3′ portion comprising the sequence all or partially complementary to the transposon end sequence.

In some embodiments, the fragments remain attached to a transposome at one or both end when removing the sequence all or partially complementary to the transposon end sequence.

In some embodiments, each primer comprises a different UMI. In some embodiments, the full-length RNA comprises a pool of different full-length RNAs and the polyT primer comprises a pool of different polyT primers comprising different UMIs. In some embodiments, each polyT primer comprised in the pool of different polyT primers comprises a different UMI.

In some embodiments, each fragment comprises a unique UMI. In some embodiments, the full-length RNA comprises a pool of different full-length RNAs and the 3′ double-stranded DNA fragment prepared from a single full-length RNA comprises a UMI that is different from the 3′ double-stranded DNA fragments prepared from other full-length RNAs in the pool.

In some embodiments, each bead comprises a unique bead code. In some embodiments, the full-length RNA comprises a pool of different full-length RNAs and the bead comprises a pool of beads. In some embodiments, each bead has immobilized a transposome complexes comprising a different bead code as compared to the bead code comprised in transposome complexes immobilized on other beads in the pool.

In some embodiments, all the fragments prepared from a double-stranded cDNA prepared from a single full-length RNA are tagmented on the same bead.

In some embodiments, all the double-stranded fragments comprising the first-read sequencing adapter and the second-read sequencing adapter prepared from a double-stranded cDNA are on the same solid support after performing gap-filling and extension.

In some embodiments, the full-length RNA comprises a pool of different full-length RNAs and all the double-stranded fragments comprising the first-read sequencing adapter and the second-read sequencing adapter prepared from a single full-length RNA in the pool are on the same solid support after performing gap-filling and extension.

In some embodiments, the method further comprises amplifying the double-stranded fragments comprising the first-read sequencing adapter and the second-read sequencing adapter to prepare amplified fragments.

In some embodiments, the double-stranded cDNA preparation is by a stranded method. In some embodiments, the presence of a bead code in a sequence obtained a double-stranded fragment comprising the first-read sequencing adapter and the second-read sequencing adapter or amplified fragments identifies the bead on which the fragment was generated.

In some embodiments, the sample is a single cell.

In some embodiments, the method includes stranded RNA library preparation.

In some embodiments, one sequencing adapter sequence is incorporated into fragments during a tagmentation step and a different sequencing adapter sequence is incorporated into fragments via a primer used for elongation after tagmentation. In some embodiments, the primer that incorporates a sequencing adapter sequence is a tagged primer that comprises a sequencing adapter sequence.

In some embodiments, the present method comprises a symmetrical tagmentation step, wherein all transposome complexes comprise the same adapter sequence.

In some embodiments, the present method is compatible with 3′ UMI tagging, preamplification, and/or full-length RNA isoform detection.

In some embodiments, the method further comprises sequencing the amplified fragments or the double-stranded fragments comprising the first-read sequencing adapter and the second-read sequencing adapter.

In some embodiments, the sequencing allows full-length RNA isoform detection.

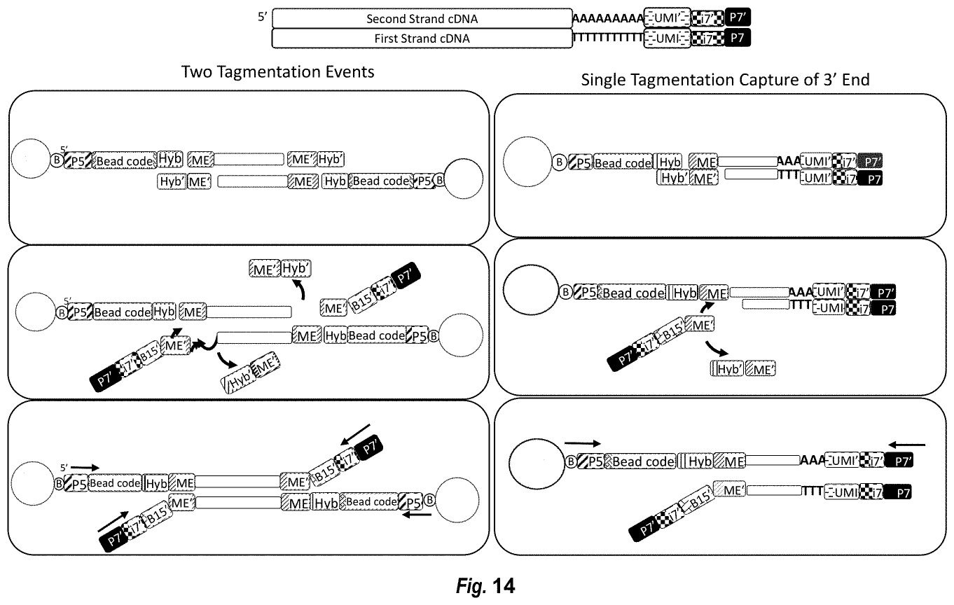

In some embodiments, the 3′ UMI (comprised in the 3′ fragment generated during tagmentation) can be used during analysis of sequencing results to identify a cDNA that is different from other cDNAs (based on other cDNAs having other UMIs). FIG. 14 outlines how the 3′ fragment incorporates a UMI during first strand synthesis (based on the UMI comprised in the first strand synthesis primer), which is then followed by a single tagmentation event after capture of the 3′ end a cDNA. Since all fragments from a given cDNA will be fragmented on the same DNA BLT, all the fragments will incorporate the same bead code. FIG. 15 summarizes this aspect of the embodiment, “UMI 1” is incorporated into cDNA from Isoform #1 during cDNA synthesis and all fragments of this isoform will incorporate “Bead Code A,” as all the fragments were generated by tagmentation on a bead comprising this code. In contrast, “UMI 2” is incorporated into the cDNA from Isoform #2 during cDNA synthesis and all fragments of this isoform will incorporate “Bead Code B,” as all the fragments were generated by tagmentation on a bead comprising this code. A method using template switch and PCR amplification is shown in FIG. 16, wherein the template switch primer can be used to incorporate an adapter (such as P5).

Using these methods, all the fragments generated from a cDNA from a given mRNA isoform can be grouped during analysis of the sequencing results. This analysis allows differentiation of different mRNA isoforms within the sequencing results.

A. Linked Long Read Sequencing

Standard short read sequencing provides accurate base level sequence to provide short range information, but short read sequencing may not provide long range genomic information. Further, because haplotype information is not retained for the sequenced genome or the reference with short read data, the reconstruction of long-range haplotypes is challenging with standard methods. As such, standard sequencing and analysis approaches generally can call single nucleotide variants (SNVs), but these methods may not identify the full spectrum of structural variation seen in an individual genome. “Structural variations” of a genome, as used herein, refers to events larger than a SNV, including events of 50 base pairs or more. Representative structural variants include copy-number variations, inversions, deletions, and duplications.

“Linked long read sequencing” or “linked-read sequencing” refers to sequencing methods that provide long range information on genomic sequences.

In some embodiments, linked-read sequencing can be used for haplotype reconstruction. In some embodiments, linked-read sequencing improves calling of structural variants. In some embodiments, linked-read sequencing improves access to region of the genome with limited accessibility. In some embodiments, linked-read sequencing is used for de novo diploid assembly. In some embodiments, linked-read sequencing improves sequencing of highly polymorphic sequences (such as human leukocyte antigen genes) that require de novo assembly.

In some embodiments, linked long-read sequencing can be performed based on spatial separation before release of fragments from a BLT or based on bead barcoding.

1. Linked Long-Read Sequencing Based on Spatial Separation

In some embodiments, a full-length nucleic acid is “wrapped” on a single bead, such as a BLT, meaning that the full-length nucleic acid can associate with multiple transposome complexes on a single bead. As used herein, the nucleic acid may be DNA, cDNA, or a DNA:RNA duplex.

In some embodiments, a bead is delivered to a surface for sequencing with a full-length nucleic acid attached to the bead. For example, a nucleic acid could be bound to an activatable BLT and delivered to a flowcell. The BLT could be activated after attaching to the flowcell to allow for preparation of fragments. The fragments could then be released, such that fragments generated from a given full-length nucleic acid (which are prepared on the same bead) would be released in close proximity, as compared to fragments prepared on other beads.

In some embodiments, a BLT is delivered to a surface for sequencing with fragments attached to the BLT.

In some embodiments, linked-read sequencing uses molecular barcodes to tag reads that come from the same long DNA fragment. When unique barcodes are added to every short read generated from an individual DNA molecule, the short reads can that DNA molecule can be linked together. In other words, reads that share a barcode can be grouped as deriving from a single long input molecule allowing long range information to be assembled from short reads.

B. First Strand Synthesis Primer

In some embodiments, a first strand synthesis primer is capable of incorporating one or more tag into the first strand of cDNA generated from an RNA comprised in a sample. In some embodiments, the first strand synthesis primer comprises a polyT sequence. In some embodiments, this polyT sequence can hybridize to the poly-A tail on the 3′ end of an RNA. In some embodiments, the RNA is mRNA. In some embodiments, use of a primer comprising a polyT sequence allows tagging of a first stand of cDNA with a 3′ UMI.

In some embodiments, the first strand synthesis primer further comprises a UMI, an index sequence (or its complement), a first-read sequencing adapter sequence (or its complement), and/or one or more additional adapter sequence.

In some embodiments, a first strand synthesis primer is comprised in a pool of first strand synthesis primers. In some embodiments the first strand synthesis primer in a pool of first strand synthesis primers comprises a unique UMI, which is different from all or most other primers in the mix.

In some embodiments, the first strand synthesis primer comprises an oligo dT sequence, a UMI, an index sequence, and an adapter sequence. A representative first strand of cDNA generated with such a primer is shown in FIG. 14, wherein the first strand of cDNA comprises an oligo dT sequence, a UMI, an i7 sequence (an index sequence), and a P7 sequence (i.e., an adapter sequence used as a primer landing site). In some embodiments, a surface for sequencing, such as a flowcell, is coated with a lawn of primer landing sites. In some embodiments a primer landing site is P5 or P7. In some embodiments, a primer landing site (such as P5 or P7) facilitates binding of fragments to a flowcell.

In some embodiments, the oligo dT sequence and the first-read sequencing adapter sequence are identical for each first strand synthesis primer in a mix of primers. In some embodiments, the UMI is unique for each first strand synthesis primer. In this way, downstream sequencing events can differentiate fragments generated from different RNA molecules comprised in a sample comprising different RNAs.

In some embodiments, the index sequence comprised in a first strand synthesis primer is one of a known pool of index sequences, such as i7 or i5 sequences (See, for example, Illumina Document #1000000002694 v10, Illumina, Inc. 2019).

In some embodiments, the first strand synthesis primer comprises one or more adapter sequence. In some embodiments, the adaptor sequence comprises a primer sequence, an index tag sequence, a capture sequence, a barcode sequence, a cleavage sequence, or a sequencing- related sequence, or a combination thereof.

C. Unique Molecular Identifiers (UMIs)

Unique molecular identifiers (UMIs) are sequences of nucleotides applied to or identified in nucleic acid molecules that may be used to distinguish individual nucleic acid molecules from one another. UMIs may be sequenced along with the nucleic acid molecules with which they are associated to determine whether the read sequences are those of one source nucleic acid molecule or another. The term “UMI” may be used herein to refer to both the sequence information of a polynucleotide and the physical polynucleotide per se. UMIs are similar to barcodes, which are commonly used to distinguish reads of one sample from reads of other samples, but UMIs are instead used to distinguish nucleic acid template fragments from another when many fragments from an individual sample are sequenced together. UMIs may be defined in many ways, such as described in WO 2019/108972 and WO 2018/136248, which are incorporated herein by reference.

Unique molecular identifiers (UMIs) are sequences of nucleotides applied to or identified in nucleic acid molecules that may be used to distinguish individual nucleic acid molecules from one another. UMIs may be sequenced along with the nucleic acid molecules with which they are associated to determine whether the read sequences are those of one source nucleic acid molecule or another. The term “UMI” may be used herein to refer to both the sequence information of a polynucleotide and the physical polynucleotide per se. UMIs are similar to bar codes, which are commonly used to distinguish reads of one sample from reads of other samples, but UMIs are instead used to distinguish nucleic acid template fragments from another when many fragments from an individual sample are sequenced together. UMIs may be defined in many ways, such as described in WO 2019/108972 and WO 2018/136248, which are incorporated herein by reference.

In some embodiments, the library of UMIs comprises nonrandom sequences. In some embodiments, nonrandom UMIs (nrUMIs) are predefined for a particular experiment or application. In certain embodiments, rules are used to generate sequences for a set or select a sample from the set to obtain a nrUMI. For instance, the sequences of a set may be generated such that the sequences have a particular pattern or patterns. In some implementations, each sequence differs from every other sequence in the set by a particular number of (e.g., 2, 3, or 4) nucleotides. That is, no nrUMI sequence can be converted to any other available nrUMI sequence by replacing fewer than the particular number of nucleotides. In some implementations, a set of UMIs used in a sequencing process includes fewer than all possible UMIs given a particular sequence length. For instance, a set of nrUMIs having 6 nucleotides may include a total of 96 different sequences, instead of a total of 4^{A}6=4096 possible different sequences. In some embodiments, the library of UMIs comprises 120 nonrandom sequences.

In some implementations where nrUMIs are selected from a set with fewer than all possible different sequences, the number of nrUMIs is fewer, sometimes significantly so, than the number of source DNA molecules. In such implementations, nrUMI information may be combined with other information, such as virtual UMIs, read locations on a reference sequence, and/or sequence information of reads, to identify sequence reads deriving from a same source DNA molecule.

In some embodiments, the library of UMIs may comprise random UMIs (rUMIs) that are selected as a random sample, with or without replacement, from a set of UMIs consisting of all possible different oligonucleotide sequences given one or more sequence lengths. For instance, if each UMI in the set of UMIs has n nucleotides, then the set includes 4^{A}n UMIs having sequences that are different from each other. A random sample selected from the 4^{A}n UMIs constitutes a rUMI.

In some embodiments, the library of UMIs is pseudo-random or partially random, which may comprise a mixture of nrUMIs and rUMIs.

In some embodiments, adapter sequences or other nucleotide sequences may be present between the UMI and the insert DNA.

In some embodiments, adapter sequences or other nucleotide sequences may be present between each UMI and the insert DNA.

In some embodiments, the UMI is located 3′ of the insert DNA. In some embodiments, a sequence of nucleic acids representing one or more adapter sequences may be located between the UMI and the insert DNA.

In some embodiments, the UMI is on the first strand of synthesized cDNA. In some embodiments, a first copy of the UMI is on the first strand of synthesized cDNA and a second copy of the UMI (i.e., its complement) is on the second strand of the synthesized cDNA.

D. Primer for Incorporating One or More Adapter After Tagmentation

In some embodiments, a primer is hybridized after tagmentation on a BLT to incorporate one or more adapter sequences. In some embodiments, this primer comprises a sequencing adapter sequence and a sequence all or partially complementary to the transposon end sequence. In some embodiments, the primer comprises a sequencing adapter sequence that is different from a sequencing adapter comprised in the first strand synthesis primer. In some embodiments, the sequence of the first strand synthesis primer is different from the sequence of the primer used after tagmentation.

In some embodiments the primer hybridized after tagmentation is not fully complementary to a sequence in the first transposon. In other words, hybridization of the primer to fragments immobilized to a BLT generate a “Y-adapter”- or “forked adapter”-like structure.

In some embodiments, the primer hybridized after tagmentation comprises a sequence that is all or partially complementary to a sequence comprised in the first transposon. In some embodiments, the primer hybridized after tagmentation comprises a sequence that is all or partially complementary to a mosaic end (ME) sequence (or its complement) comprised in the first transposon. In some embodiments, the primer hybridized after tagmentation comprises sequences that are not comprised in the first transposon.

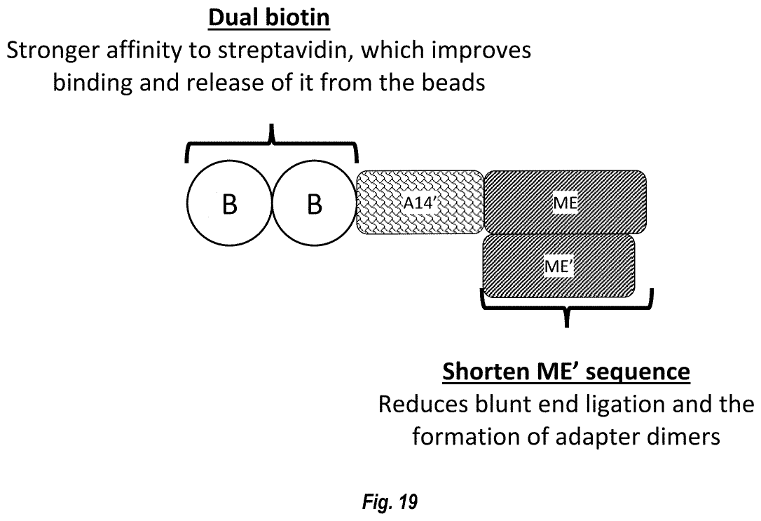

In some embodiments, a ME′ sequence in a non-transferred strand is dissociated from a fragment before hybridizing a primer comprising a sequence that is all or partially complementary to a sequence comprised in the first transposon. In some embodiments, the sequence all or partially complementary to the transposon end sequence is shorter than the transposon end sequence. Such an embodiment is shown as the shortened ME′ in FIG. 19. In some embodiments, fewer adapter dimers are generated when the sequence all or partially complementary to the transposon end sequence is shorter than the transposon end sequence (i.e., a shortened ME′ sequence is used).

In some embodiments, a second transposon comprises a shorter ME′ to facilitate dissociation of the ME′ sequence from the ME sequence after tagmentation. In some embodiments, a shortened ME′ sequence is useful for exchanging a non-transferred ME′ sequence with a different oligonucleotide (such as a primer). In some embodiments, a shortened ME′ sequence may also reduce the occurrence of blunt-end ligation.

E. Hybridization Sequence

In some embodiments, beads comprise an oligonucleotide that can be used to bind transposomes in solution. In this way, the bead may be an “activatable BLT,” wherein the user controls the timing of generation of the BLT.

In some embodiments, the bead comprises an oligonucleotide comprising a hybridization sequence. In some embodiments, the hybridization sequence binds to a sequence all or partially complementary to a sequence comprised in a transposome complex. In some embodiments, the hybridization sequence binds to the second transposon comprised in transposomes, wherein transposomes from solution can be bound (to form BLTs) via the hybridization sequence that is all or partially complementary to a sequence comprised in the second transposon

F. Bead Code

The 3′ fragment of a given cDNA can incorporate (as described above) a UMI sequence. In this way, the UMI sequence in the 3′ fragment generated from a given cDNA can be used to differentiate this cDNA from others that are generated. This 3′ fragment generated from a given cDNA (as shown in FIG. 15) or DNA:RNA duplex would also comprise a bead code incorporated during tagmentation. In this way, sequencing data from the 3′ fragment (comprising a UMI) could be sorted together with sequencing data from other fragments generated on the same bead to generate a full sequence of the starting RNA from the sample. In other words, a cDNA generated from a full-length RNA transcript will be tagmented by a single bead, which tagments all segments of the cDNA from the original full-length RNA with identical bead code sequences. In some embodiments, fragmenting a single cDNA on a given bead allows all fragments to be linked back to the original transcript as well as to the UMI introduced during reverse transcription. Accordingly, this method can provide data on unique RNA molecules.

While all fragments of a given cDNA would generally be generated on the same BLT, this BLT could also generate fragments from other cDNA(s) that bind to it. In other words, fragments that originate from different RNAs in the original sample could have the same bead code. However, the sequence alignments can be used to identify which fragments on a given bead originated from a given sequence.

### IV. Additional Methods of Preparing RNA or DNA Libraries After Symmetric Tagmentation

A number of different ways to generate sequencable fragments may be used after a symmetrical tagmentation on a BLT. These methods may be combined with any of the protocols described herein. A number of exemplary methods are disclosed in US Provisional Application No. 63/168,802, which is incorporated herein in its entirety.

In some embodiments, a method comprises, after tagmentation of DNA or DNA:RNA duplexes, releasing the double-stranded target nucleic acid fragments from the transposome complex, hybridizing a polynucleotide comprising an adapter sequence and a sequence all or partially complementary to the first 3′ end transposon sequence, wherein the adapter sequence comprised in the polynucleotide is different from the adapter sequence comprised in the transposome complexes, optionally extending a second strand of the double-stranded target nucleic acid fragments, optionally ligating the polynucleotide or extended polynucleotide with the double- stranded target nucleic acid fragments, and producing double-stranded target nucleic acid fragments. In some embodiments, the polynucleotide further comprises a UMI. In some embodiments, fragments comprise the UMI, wherein the UMI is located directly adjacent to the 3′ end of the insert DNA.

In some embodiments, a method comprises, after tagmentation of DNA or DNA:RNA duplexes, releasing the double-stranded target nucleic acid fragments from the transposome complex, hybridizing a first polynucleotide an adapter sequence, wherein the adapter in the first transposon is different from the adapter in the first polynucleotide, optionally adding a second polynucleotide comprising regions complementary to the first polynucleotide to produce a double-stranded adapter, optionally extending a second strand of the double-stranded target nucleic acid fragments, optionally ligating the double-stranded adapter with the double-stranded target nucleic acid fragments, and producing double stranded target nucleic acid fragments. In some embodiments, the first polynucleotide further comprises a UMI. In some embodiments, the fragments comprise a UMI, wherein the UMI is located between the double-stranded target nucleic acid fragments and the adapter sequence from the first polynucleotide.

In some embodiments, fragments are tagged with a first-read sequence adapter sequence from the first transposon at the 5′ end of one strand and with a second-read sequence adapter sequence from the first polynucleotide at the 5′ end of the other strand.

V. Methods of Preparing Strand-Specific cDNA Preparation with Tagmentation for Library Preparation (PRESS-BLT)

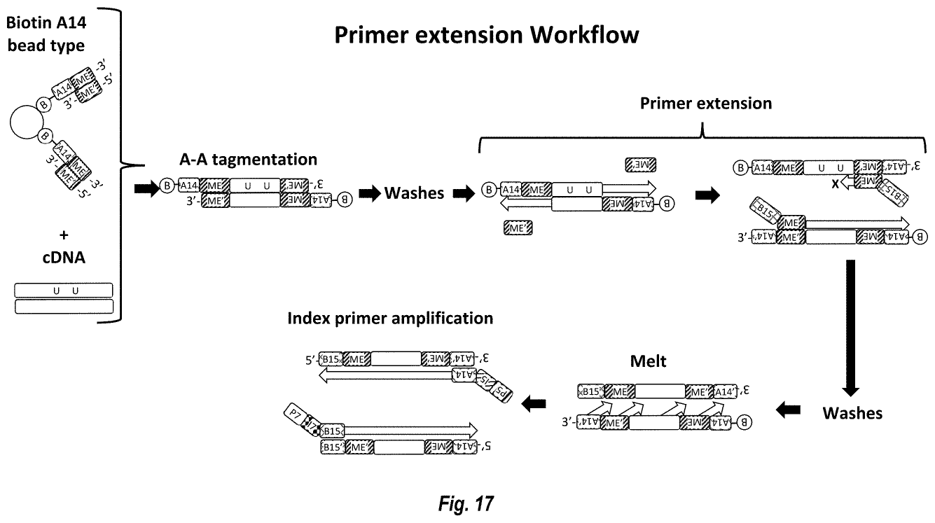

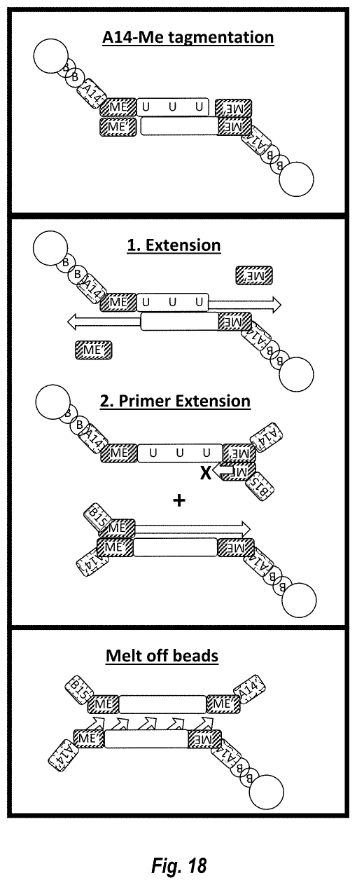

In some embodiments, a method of strand-specific cDNA preparation is combined with tagmentation to prepare libraries. Primer extension strand-specific BLT approach (PRESS-BLT) may be used to describe a strand-specific method comprising cDNA synthesis, BLT formulation, and library preparation, as shown in FIGS. 17 and 18.

In some embodiments, method of preparing strand-specific libraries of single- stranded DNA from RNA via PRESS-BLT comprises preparing a first strand of cDNA from an RNA comprised in a sample using a reverse transcriptase, a primer, and nucleotides comprising dTTP under conditions that inhibit DNA-dependent DNA synthesis; preparing a second strand of cDNA from the first strand of cDNA using a DNA polymerase, a primer, and nucleotides comprising dUTP to prepare double-stranded cDNA; applying the double-stranded cDNA to a solid support having transposome complexes immobilized thereon, wherein each transposome complex comprises a transposase; a first transposon comprising a 3′ portion comprising a transposon end sequence and a first-read sequencing adapter sequence; wherein the first transposon comprises a 5′ affinity element for immobilizing the transposome complex to the solid support; and a second transposon sequence comprising a sequence all or partially complementary to the transposon end sequence; fragmenting the double-stranded DNA with the transposome complexes to prepare tagged double-stranded DNA fragments comprising the first-read sequencing adapter sequence; removing the second transposon and performing gap-filling and extension; separating the strands of the double-stranded DNA fragments; hybridizing a primer comprising a second-read sequencing adapter sequence to the transposon end sequence or the sequence all or partially complementary to the transposon end sequence and amplifying to prepare a DNA strand that is not attached to the solid support and that comprises the first-read sequencing adapter and the second-read sequencing adapter; and releasing the strand generating by the amplifying from the solid support, wherein the releasing releases a single-stranded DNA fragment comprising the first-read sequencing adapter and the second-read sequencing adapter.

In some embodiments, the conditions that inhibit DNA-dependent DNA synthesis is the presence of a buffer comprising actinomycin D. In some embodiments, the primer is one or more randomer primers. In some embodiments, the primer is a mix of a randomer primer and a polyT primer. In some embodiments, the primer for the preparing a second strand of cDNA is the same as the primer for the preparing a first strand of cDNA. In some embodiments, the RNA is a long non-coding RNA or antisense transcript.

In some embodiments, the amplifying is performed with a uracil-intolerant polymerase. In some embodiments, the amplifying does not amplify from a DNA strand comprising uracil.

In some embodiments, a unique molecular identifier (UMI) is comprised in the primer comprising a second-read sequencing adapter sequence. In some embodiments, the UMI is located between the second-read sequencing adapter sequence and the sequence that can bind to the transposon end sequence or the sequence all or partially complementary to the transposon end sequence. In some embodiments, a UMI is comprised in the first transposon. In some embodiments, the UMI is located between the transposon end sequence and the first-read sequencing adapter sequence.

In some embodiments, different fragments in the resulting library comprise different UMIs. In some embodiments, the RNA comprises a pool of different RNAs and the single-stranded fragment comprising the first-read sequencing adapter and the second-read sequencing adapter comprises a pool of different fragments, wherein each fragment comprises a UMI that is different from other fragments comprised in the pool of different fragments.

A range of different affinity elements may be used in the PRESS-BLT method. In some embodiments, the affinity element is a biotin or desthiobiotin and the solid support comprises streptavidin or avidin on its surface. In some embodiments, the affinity element is a dual biotin, as described in FIG. 19.

Releasing the strand generating by the amplifying from the solid support may be performed in a number of ways. In some embodiments, the releasing is performed with heat or sodium hydroxide treatment. In some embodiments, the single-stranded fragment comprising the first-read sequencing adapter and the second-read sequencing adapter is partitioned from the solid support after the releasing.

In some embodiments, the method further comprises performing index primer amplification with the single-stranded DNA fragment comprising the first-read sequencing adapter and the second-read sequencing adapter to prepare an indexed fragment after the releasing. Such index primer amplification is well-known in the art for indexing of sequencing data. In some embodiments, the index primer amplification is performed in a separate reaction vessel from the solid support.

In some embodiments, the index primer amplification is performed with a uracil-intolerant polymerase. In this way, second strands of cDNA are not amplified if they comprise uracil.

In some embodiments, the method further comprises sequencing the single-stranded DNA fragment comprising the first-read sequencing adapter and the second-read sequencing adapter or the indexed fragment. In some embodiments, sequencing data is generated from the first strand of cDNA generated from the RNA. In some embodiments, sequencing data is not generated from the second strand of cDNA generated from the RNA. In some embodiments, the method does not require ligation.

PRESS-BLT provides a number of advantages for mRNA analysis. In some embodiments, the method demarcates the boundaries of overlapping sequences in the RNA. In some embodiments, the method allows estimate of transcript expression. In some embodiments, the estimate of transcript expression is based on analysis of UMIs. A general overview of PRESS-BLT is that it comprises steps of strand-specific cDNA synthesis, symmetric tagmentation by BLTs, and primer extension to prepare library fragments.

A. Strand-Specific cDNA Synthesis in PRESS-BLT

In some embodiments, total RNA is copied into first strand cDNA using reverse transcriptase, random primers, nucleoside triphosphates, and a FSA buffer that includes Actinomycin D. In some embodiments, actinomycin D specifically inhibits DNA-dependent DNA synthesis and improves strand specificity. A representative method of strand-specific cDNA synthesis is shown in FIG. 17.

Such strand-specific cDNA synthesis may be used within a PRESS-BLT method, but also may be used with other methods of library preparation.

B. BLTs for Use in PRESS-BLT

In some embodiments, double-stranded cDNA prepared using a strand- specific protocol is then tagmented to generate tagged double-stranded DNA fragments.

In some embodiments, the tagmentation in a PRESS-BLT method is symmetrical tagmentation, wherein all transposome complexes comprise a first transposon comprising the same adapter sequence. In some embodiments, methods using a symmetrical tagmentation step increases yield of sequencable fragments (i.e., each fragment having a different sequencing adapter sequence at each end of the fragment) as compared to asymmetrical tagmentation steps wherein more than one type of transposome complex is used for tagmentation.

In some embodiments, the cDNA is tagmented such that fragments incorporate the same tag at the 5′ end of both strands. In some embodiments, the tagged double- stranded cDNA fragments generated by tagmentation have the same tag at both ends of the fragments. In some embodiments, the cDNA is tagmented with a tag comprising a single sequencing adapter sequence. In some embodiments the sequencing adapter sequence is A14 or B15.

C. Primer Extension in PRESS-BLT

In some embodiments, non-transferred ME′ sequences (from the second transposon) are melted off and gap-filled by PCR extension after tagmentation. In some embodiments, the ME′ sequences are removed by raising the temperature of the reaction. These steps of the method are outlined in FIG. 18.

In some embodiments, gap-filling is performed after non-transferred ME′ sequences are removed. In some embodiments, a primer is annealed to the gap-filled ME′ sequence. In some embodiments, this primer is used for extension and may be referred to as an extension primer. In some embodiments, the extension primer comprises a ME sequence. In some embodiments, the ME sequence comprised in the extension primer hybridizes to the gap-filled ME′ sequence.

In some embodiments, the extension primer also comprises a sequencing adapter sequence. In some embodiments, the sequence adapter sequence comprised in the extension primer was not comprised in the transposome complexes. In some embodiments, extension with the extension primer generates fragments comprising different sequencing adapter sequences at each end of the double-stranded fragments.

In some embodiments, a uracil-intolerant DNA is used for primer extension. In some embodiments, primer extension only occurs from the first strand of cDNA. In some embodiments, the second strand of cDNA is not extended because it comprises uracils, based on the strand-specific cDNA preparation described above.

In some embodiments, if the transposome complexes comprises an A14 sequence (or its complement), the extension primer comprises B15 (or its complement). In some embodiments, if the transposome complexes comprises an B15 sequence (or its complement), the extension comprises A14 (or its complement). In these representative examples, A14 and 15 only represent exemplary sequencing adapter sequences, and the present methods are not limited to such adapter sequences. Any set of paired adapter sequences of interest could be used in the transposome complexes and extension primers, and one skilled in the art would be well-aware of how sequencing is performed on different platforms and that such platforms may evolve over time.

In some embodiments, extension primers comprise UMIs. In some embodiments, UMIs mark unique mRNA transcripts versus copies that are produced by PCR amplification. In other words, amplicon copies from the same cDNA (originating from a single mRNA transcript, for example) from any downstream amplification steps will comprise the same UMI. In this way, analysis of sequencing results can identify multiple copies of fragments generated from the same mRNA.

As shown in FIG. 19, certain modified transposons may improve results of PRESS-BLT. These modifications may also be useful in other methods described herein. In some embodiments, dual biotin is used to immobilize a transposon sequence to a bead to generate a BLT. In some embodiments, dual biotin has stronger affinity for streptavidin as compared to biotin, which improves the binding and release of transposomes from a BLT. Such modifications may be used in the PRESS-BLT method, along with any other method described herein.

### VI. Methods of Preparing RNA and DNA Sequencing Libraries Using Bead-Linked Transposomes

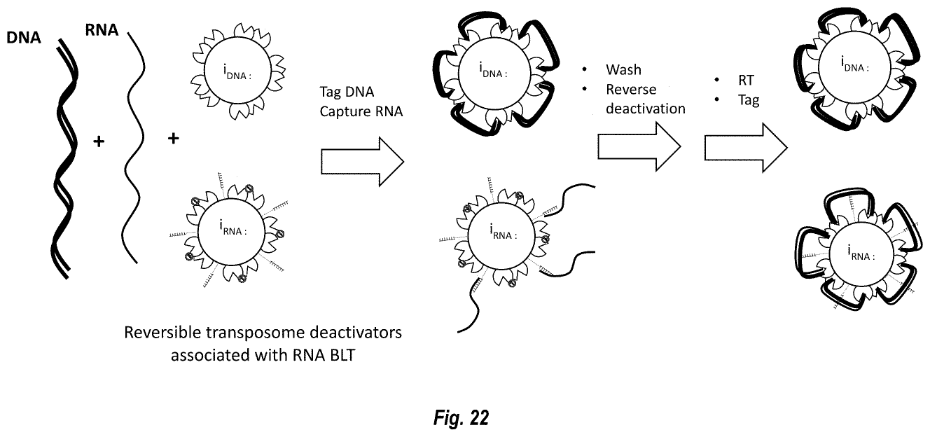

In some embodiments, methods apply a sample comprising RNA and DNA. These methods can be performed with similar components as methods recited for using RNA sequencing libraries. Any of the methods for RNA sequencing shown in FIGS. 2-18 and described herein may be used when preparing RNA and DNA sequencing libraries from the same sample. FIGS. 22-28 also show exemplary methods wherein RNA may be converted to a DNA:RNA duplex or to double-stranded DNA during the method.

In some embodiments, the present methods can resolve sequencing of samples originating from RNA and samples originating from DNA of a total nucleic acid (TNA) sample through tagmentation-based library preparation. In some embodiments, method allow a single reaction vessel to generate RNA and DNA libraries.

In some embodiments, methods allow “directional” library preparation that can differentiate the strand of nucleic acid that was the origin of a sequenced fragment.

In some embodiments, aspects of different methods can be combined to generate “stranded” RNA and DNA tagmentation-based sequencing libraries.

In some embodiments, DNA and RNA sequencing libraries can be generated from a single sample reaction. In some embodiments, DNA and RNA sequencing libraries can be generated in a single reaction vessel. In some embodiments, the present methods can capture genomic and transcriptomic or other information in one reaction, which may be referred to as a multi-omic assay.

A variety of methods can be used with samples comprising RNA and DNA to allow preparation of RNA and DNA sequencing libraries from the same sample. In some embodiments, these methods avoid tagmentation of the DNA by the RNA-BLT. Instead, the RNA in a sample comprising RNA and DNA is tagmented by BLTs designed for DNA:RNA duplex fragmentation (RNA BLTs) and the DNA in the sample is tagmented by BLTs designed for DNA fragmentation (DNA BLTs).

FIG. 21 presents the challenge in these methods, namely that DNA can itself bind to transposomes that would be present on RNA BLTs. This application describes a number of methods to specify tagmentation of DNA:RNA duplexes by RNA BLTs. These methods avoid tagmentation of DNA by RNA BLTs. FIGS. 22-28 provide exemplary workflows to allow for tagmentation of DNA by DNA BLTs (such as to incorporate a DNA-specific barcode) and tagmentation of DNA:RNA duplexes or double-stranded DNA prepared from RNA by RNA BLTs (such as to incorporate an RNA-specific barcode).

In some embodiments, a first tag and a second tag are different. In some embodiments, the first and second tags allow for differentiation of fragments of the RNA sequencing library from fragments of the DNA sequencing library. In some embodiments, an index for identifying fragmentation by a DNA BLT is termed an “iDNA” or an index identifying DNA BLT (See FIG. 21). In some embodiments, an index for identifying fragmentation by an RNA BLT is termed an “iRNA” or an index identifying RNA BLT (See FIG. 21).

The challenge with such workflows is to direct double-stranded DNA substrate to just the DNA BLT and not the RNA BLT. FIG. 21 summarizes that DNA molecules will have affinity for RNA BLTs due to the ability to bind directly to transposome complexes. The present methods describe a variety of ways to efficiently direct RNA fragmentation to RNA BLTs and DNA fragmentation to DNA BLTs when a sample comprises both RNA and DNA.

A. Methods Using 3 Beads

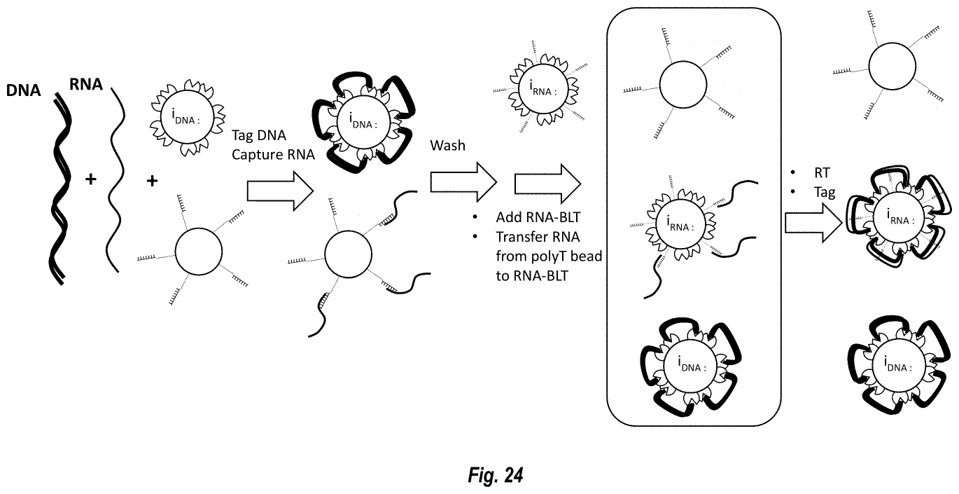

In some embodiments, a method of preparing an immobilized library of tagged DNA:RNA fragments from a sample comprising RNA and DNA uses 3 beads. An exemplary method with 3 beads is shown in FIG. 24. In some embodiments, these 3 beads may be RNA BLTs, DNA BLTs, and “RNA capture beads.” The RNA capture beads may comprise polyT capture oligonucleotides or another agent to capture RNA, without leading to RNA tagmentation (i.e., the RNA capture beads lack active transposome complexes). After DNA tagmentation and capture of RNA by RNA capture beads, the RNA is transferred from the RNA capture beads to the RNA BLTs.

In some embodiments, a method of preparing an immobilized library of tagged DNA:RNA fragments from a sample comprising RNA and DNA, comprises
applying the sample comprising RNA and DNA to a first solid support for immobilizing DNA comprising first transposome complexes immobilized thereon, wherein the first transposome complexes comprise a transposase and a first polynucleotide comprising a 3′ portion comprising a transposon end sequence, and optionally a first tag; and
a second solid support having first capture oligonucleotides immobilized thereon; wherein the sample is applied to the mixture of first and second solid supports under conditions wherein the DNA binds to the first transposome complexes on the first solid support and is fragmented and optionally tagged, and the RNA binds to the first capture oligonucleotides on the second solid support;
transferring the RNA bound to the second solid support to a third solid support having second capture oligonucleotides that bind the transferred RNA and second transposome complexes immobilized thereon, wherein the second transposome complexes comprise a transposase and a second polynucleotide comprising a 3′ portion comprising a transposon end sequence, and a second tag;
adding a reverse transcriptase polymerase under conditions to synthesize cDNA and generate immobilized DNA:RNA duplexes on the second capture oligonucleotides; and
fragmenting the DNA:RNA duplexes with the second transposome complexes under conditions wherein the DNA:RNA duplexes are tagged on the 5′ end of one strand, thereby producing an immobilized library of DNA:RNA fragments wherein at least one strand is 5′-tagged with the first tag. In some embodiments, the 5′ end of one strand is the 5′ end of the RNA strand. In some embodiments, the 5′ end of one strand is the 5′ end of the DNA strand.

In some embodiments, the first and/or second capture oligonucleotides comprise a polyT sequence. In some embodiments, the RNA comprises a sequence complementary to at least a portion of one or more of the first and/or second capture oligonucleotides. In some embodiments, the first and/or second transposome complexes are immobilized to the solid support via the first and/or second polynucleotides. In some embodiments, the method further comprises washing the solid support after applying the sample to the solid supports to remove any unbound DNA or RNA.

B. Methods Using 2 Beads: DNA BLTs and RNA Beads Lacking Functional Transposomes

In some embodiments, a method of preparing an immobilized library of tagged DNA:RNA fragments from a sample comprising RNA and DNA uses 2 solid supports. In some embodiments, the solid supports are beads. In some embodiments, the 2 beads are DNA BLTs and “naked” RNA beads. A “naked” BLT, as used herein, refers to a bead that can link transposomes complexes, but which does not have active transposomes complexes linked. For example, the transposome complexes may lack transposases or another essential component needed for activity of the transposome complexes. Thus, a “naked” BLT allows for an additional component to be added during a later step to allow fragmentation. Use of a “naked” RNA BLT allows for control of the timing of RNA fragmentation.

In some embodiments, the first solid support for immobilizing DNA comprises first transposome complexes immobilized thereon, wherein the first transposome complexes comprise a transposase and a first polynucleotide comprising a 3′ portion comprising a transposon end sequence. In some embodiments, the first solid support further comprises a first tag.

In some embodiments, a second solid support has capture oligonucleotides and a second polynucleotide immobilized thereon, wherein the second polynucleotide comprises a 3′ portion comprising a transposon end sequence and a second tag. In some embodiments, the second tag is an RNA-specific barcode.

In some embodiments, a method of preparing an immobilized library of tagged DNA:RNA fragments from a sample comprising RNA and DNA, comprises:
applying a sample comprising RNA and DNA to a first solid support for immobilizing DNA comprising first transposome complexes immobilized thereon, wherein the first transposome complexes comprise a transposase and a first polynucleotide comprising a 3′ portion comprising a transposon end sequence, and optionally a first tag; and
a second solid support having capture oligonucleotides and a second polynucleotide immobilized thereon, wherein the second polynucleotide comprises a 3′ portion comprising a transposon end sequence and a second tag; wherein the sample is applied to the mixture of first and second solid supports under conditions wherein the DNA binds to the first transposome complexes on the first solid support and is fragmented and optionally tagged, and the RNA binds to the capture oligonucleotides on the second solid support;
adding a transposase under conditions wherein the transposase binds to the second polynucleotide to form transposome complexes on the second solid support;
adding a reverse transcriptase polymerase under conditions to synthesize cDNA and generate immobilized DNA:RNA duplexes on the second capture oligonucleotides; and
fragmenting the DNA:RNA duplexes with the second transposome complexes under conditions wherein the DNA:RNA duplexes are tagged on the 5′ end of one strand, thereby producing an immobilized library of DNA:RNA fragments wherein at least one strand is 5′-tagged with the first tag. In some embodiments, the 5′ end of one strand is the 5′ end of the RNA strand. In some embodiments, the 5′ end of one strand is the 5′ end of the DNA strand.

In some embodiments, the first and/or second capture oligonucleotides comprise a polyT sequence. In some embodiments, the RNA comprises a sequence complementary to at least a portion of one or more of the first and/or second capture oligonucleotides. In some embodiments, the first and/or second transposome complexes are immobilized to the solid support via the first and/or second polynucleotides. In some embodiments, the method further comprises washing the solid support after applying the sample to the solid supports to remove any unbound DNA or RNA.

C. Methods Using 2 Beads: DNA BLTs and Deactivated RNA BLTs

In some embodiments, a method of preparing an immobilized library of tagged DNA:RNA fragments from a sample comprising RNA and DNA uses 2 solid supports. In some embodiments, the solid supports are beads. In some embodiments, the 2 beads are DNA BLTs and “deactivated” RNA BLTs. As used herein, “deactivated” RNA BLTs refers to RNA BLTs that are reversibly deactivated. Thus, a “deactivated” RNA BLT allows for activation during a later step to allow fragmentation. Use of a “deactivated” RNA bead thus allows for control of the timing of RNA fragmentation.

In some embodiments, a method of preparing an immobilized library of tagged DNA:RNA fragments from a sample comprising RNA and DNA, comprises:
applying a sample comprising RNA and DNA to a first solid support for immobilizing DNA comprising first transposome complexes immobilized thereon, wherein the first transposome complexes comprise a transposase and a first polynucleotide comprising a 3′ portion comprising a transposon end sequence, and optionally a first tag; and a second solid support for immobilizing RNA having capture oligonucleotides and second transposome complexes that are reversibly deactivated immobilized thereon, wherein the transposome complexes comprise a transposase bound to a second polynucleotide comprising a 3′ portion comprising a transposon end sequence, and a second tag; wherein the sample is applied to the mixture of first and second solid supports under conditions wherein the DNA binds to the first transposome complexes on the first solid support and is fragmented and optionally tagged, and the RNA binds to the capture oligonucleotides on the second solid support;
adding a reverse transcriptase polymerase under conditions to synthesize cDNA and generate immobilized DNA:RNA duplexes on the second capture oligonucleotides;
activating the second transposome complexes; and
fragmenting the DNA:RNA duplexes with the activated second transposome complexes under conditions wherein the DNA:RNA duplexes are tagged on the 5′ end of one strand, thereby producing an immobilized library of DNA:RNA fragments wherein at least one strand is 5′-tagged with the first tag. In some embodiments, the 5′ end of one strand is the 5′ end of the RNA strand. In some embodiments, the 5′ end of one strand is the 5′ end of the DNA strand.

In some embodiments, the transposome complex is reversibly deactivated by a transposome deactivator bound to the transposome complex. In some embodiments, the transposome deactivator is bound to a Tn5 binding site of the transposome complex. In some embodiments, the transposome deactivator comprises dephosphorylated ME′, extra bases, inhibitor duplexes, and/or heat-labile antibodies. In some embodiments, the transposome complex is activated by removal of the transposome deactivator.

In some embodiments, the capture oligonucleotides comprise a polyT sequence. In some embodiments, the RNA comprises a sequence complementary to at least a portion of one or more of the capture oligonucleotides. In some embodiments, the first and/or second transposome complexes are immobilized to the solid support via the first and/or second polynucleotides. In some embodiments, the method further comprising washing the solid support after applying the sample to remove any unbound DNA or RNA.

D. Methods Using 2 Beads With Sequential Immobilization of DNA and RNA

In some embodiments, a method of preparing an immobilized library of tagged DNA:RNA fragments from a sample comprising RNA and DNA uses 2 solid supports. In some embodiments, the solid supports are beads. In some embodiments, the DNA and RNA in a sample are sequentially immobilized on separate solid supports.

In some embodiments, a method of preparing an immobilized library of tagged DNA:RNA fragments from a sample comprising RNA and DNA, comprises applying a sample comprising RNA and DNA to a first solid support for immobilizing DNA comprising first transposome complexes immobilized thereon, wherein the first transposome complexes comprise a transposase and a first polynucleotide comprising a 3′ portion comprising a transposon end sequence, and optionally a first tag, and wherein the sample is applied under conditions wherein the DNA binds to the first transposome complexes on the first solid support and is fragmented and optionally tagged;
separating the first solid support with the bound DNA from the RNA;
applying the RNA to a second solid support for immobilizing RNA having capture oligonucleotides and second transposome complexes immobilized thereon, wherein the second transposome complexes comprise a transposase bound to a second polynucleotide comprising a 3′ portion comprising a transposon end sequence, and a second tag, and wherein the RNA is applied under conditions wherein the RNA binds to the capture oligonucleotides on the second solid support;
adding a reverse transcriptase polymerase under conditions to synthesize cDNA and generate immobilized DNA:RNA duplexes on the second capture oligonucleotides; and
fragmenting the DNA:RNA duplexes with the activated second transposome complexes under conditions wherein the DNA:RNA duplexes are tagged on the 5′ end of one strand, thereby producing an immobilized library of DNA:RNA fragments wherein at least one strand is 5′-tagged with the first tag. In some embodiments, the 5′ end of one strand is the 5′ end of the RNA strand. In some embodiments, the 5′ end of one strand is the 5′ end of the DNA strand.

In some embodiments, the capture oligonucleotides comprise a polyT sequence. In some embodiments, the RNA comprises a sequence complementary to at least a portion of one or more of the capture oligonucleotides. In some embodiments, the first and/or second transposome complexes are immobilized to the solid support via the first and/or second polynucleotides. In some embodiments, the method further comprises washing the solid support after step applying the RNA to remove any unbound RNA. In some embodiments, the method further comprises recombining the first solid support with the bound DNA with the second solid support with the immobilized library of tagged DNA:RNA fragments.

E. Methods Using 2 Beads with Sequential Immobilization of DNA and RNA and Preparation of Double-Stranded cDNA

In some embodiments, a method of preparing an immobilized library of tagged DNA:RNA fragments from a sample comprising RNA and DNA uses 2 solid supports. In some embodiments, the solid supports are beads. In some embodiments, the DNA and double-stranded cDNA (ds-cDNA) generated from RNA are sequentially immobilized on separate solid supports.

In some embodiments, the method of preparing an immobilized library of tagged fragments from a sample comprising RNA and DNA, wherein the tagged fragments comprise either a DNA-specific barcode or an RNA-specific barcode, comprises combining a sample comprising RNA and DNA with a first solid support for immobilizing DNA, wherein the first solid support comprises transposome complexes immobilized thereon, wherein the transposome complexes comprise a transposase and a transposon comprising a transposon end sequence and a DNA-specific barcode; immobilizing the DNA; performing tagmentation on the first solid support to prepare tagged fragments comprising a DNA-specific barcode; preparing double-stranded cDNA from the RNA; combining the sample with a second solid support for immobilizing cDNA, wherein the second solid support comprises transposome complexes immobilized thereon, wherein the transposome complexes comprise a transposase and a transposon comprising a transposon end sequence and an RNA-specific barcode; and immobilizing the cDNA and performing tagmentation on the second solid support to prepare tagged fragments comprising an RNA-specific barcode.

In some embodiments, the ds-cDNA is generated in solution. In some embodiments, the first and second solid supports are combined after performing tagmentation on the second solid support, wherein each solid support has immobilized tagged fragments comprising either a DNA-specific barcode or an RNA-specific barcode.

In some embodiments, the method comprises partitioning the first solid support with the immobilized tagged fragments comprising a DNA-specific barcode from the rest of the sample after performing tagmentation on the first solid support and before preparing double-stranded cDNA from the RNA.

In some embodiments, the method comprises partitioning the first solid support with the immobilized DNA from the rest of the sample after immobilizing the DNA and before performing tagmentation on the first solid support to prepare tagged fragments comprising a DNA-specific barcode.

In some embodiments, the preparing double-stranded cDNA from the RNA is performed by template switching.

F. Methods Using 2 Beads with Sequential Immobilization of DNA and RNA and Preparation of DNA:RNA Duplexes

In some embodiments, a method of preparing an immobilized library of tagged DNA:RNA fragments from a sample comprising RNA and DNA uses 2 solid supports. In some embodiments, the solid supports are beads. In some embodiments, the DNA and DNA:RNA duplexes generated from RNA are sequentially immobilized on separate solid supports.

In some embodiments, a method of preparing an immobilized library of tagged fragments from a sample comprising RNA and DNA, wherein the tagged fragments comprise either a DNA-specific barcode or an RNA-specific barcode, comprises combining a sample comprising RNA and DNA with a first solid support for immobilizing DNA, wherein the first solid support comprises transposome complexes immobilized thereon, wherein the transposome complexes comprise a transposase and a transposon comprising a transposon end sequence and a DNA-specific barcode; immobilizing the DNA; performing tagmentation on the first solid support to prepare tagged fragments comprising a DNA-specific barcode; preparing a single strand of cDNA from the RNA to produce DNA:RNA duplexes; combining the sample with a second solid support for immobilizing DNA:RNA duplexes, wherein the second solid support comprises transposome complexes immobilized thereon, wherein the transposome complexes comprise a transposase with activity on DNA:RNA duplexes and a transposon comprising a transposon end sequence and an RNA-specific barcode; and immobilizing the DNA:RNA duplexes and performing tagmentation on the second solid support to prepare tagged fragments comprising an RNA-specific barcode.

In some embodiments, the method further comprises combining the first and second solid supports after performing tagmentation on the second solid support, wherein each solid support has immobilized tagged fragments comprising either a DNA-specific barcode or an RNA-specific barcode.

In some embodiments, the method further comprises partitioning the first solid support with the immobilized tagged fragments comprising a DNA-specific barcode from the rest of the sample after performing tagmentation on the first solid support and before preparing a single strand of cDNA from the RNA to produce DNA:RNA duplexes.

In some embodiments, the method further comprises partitioning the first solid support with the immobilized DNA from the rest of the sample after immobilizing the DNA and before performing tagmentation on the first solid support to prepare tagged fragments comprising a DNA-specific barcode.

In some embodiments, the DNA:RNA duplexes are generated in solution. In some embodiments, the first and second solid supports are combined after performing tagmentation on the second solid support, wherein each solid support has immobilized tagged fragments comprising either a DNA-specific barcode or an RNA-specific barcode.

G. Methods Using 2 Beads: DNA Tagmentation with DNA BLTs and Solution-Phase cDNA or DNA:RNA Duplex Tagmentation

In some embodiments, DNA in a sample may be tagmented using DNA BLTs, and then the double-stranded cDNA or DNA:RNA duplexes prepared from the RNA are tagmented in solution. In other words, the cDNA or DNA:RNA duplexes may be reacted with solution-phase transposome complexes after preparing the tagged DNA fragments. In some embodiments, the tagmentation of the double-stranded cDNA or DNA:RNA duplexes incorporates a sequence that can hybridize to capture probes. In some embodiments, the tagged fragments generated from the cDNA or DNA:RNA duplexes can be bound by a solid support that comprises capture probes on its surface.

In some embodiments, a method of preparing an immobilized library of tagged fragments from a sample comprising RNA and DNA, wherein the tagged fragments comprise either a DNA-specific barcode or an RNA-specific barcode, comprises combining a sample comprising RNA and DNA with a first solid support for immobilizing DNA, wherein the first solid support comprises transposome complexes immobilized thereon, wherein the transposome complexes comprise a transposase and a transposon comprising a transposon end sequence and a DNA-specific barcode; immobilizing the DNA; performing tagmentation on the first solid support to prepare tagged fragments comprising a DNA-specific barcode; preparing double-stranded cDNA from the RNA; performing tagmentation on the double-stranded DNA in solution, wherein the transposome complexes in solution comprise a transposase and a transposon comprising a transposon end sequence, an RNA-specific barcode, and a sequence that hybridizes to capture probes, to prepare tagged fragments of the double-stranded cDNA, wherein the tagged fragments comprise the RNA-specific barcode and the sequence that hybridizes to capture probes; combining the sample with a second solid support having a surface comprising capture probes; and immobilizing the tagged fragments of double-stranded cDNA on the second solid support.

In some embodiments, the method further comprises combining the first and second solid supports after immobilizing the tagged fragments of double-stranded cDNA on the second solid support, wherein each solid support has immobilized tagged fragments comprising either a DNA-specific barcode or an RNA-specific barcode.

In some embodiments, the method further comprises partitioning the first solid support with the immobilized tagged fragments comprising a DNA-specific barcode from the rest of the sample after performing tagmentation on the first solid support and before double-stranded cDNA from the RNA.

In some embodiments, the method further comprises partitioning the first solid support with the immobilized DNA from the rest of the sample after immobilizing the DNA and before performing tagmentation on the first solid support to prepare tagged fragments comprising a DNA-specific barcode.

In some embodiments, a method of preparing an immobilized library of tagged fragments from a sample comprising RNA and DNA, wherein the tagged fragments comprise either a DNA-specific barcode or an RNA-specific barcode, comprises combining a sample comprising RNA and DNA with a first solid support for immobilizing DNA, wherein the first solid support comprises transposome complexes immobilized thereon, wherein the transposome complexes comprise a transposase and a transposon comprising a transposon end sequence and a DNA-specific barcode; immobilizing the DNA; performing tagmentation on the first solid support to prepare tagged fragments comprising a DNA-specific barcode; preparing a single strand of cDNA from the RNA to produce DNA:RNA duplexes; performing tagmentation on the DNA:RNA duplexes in solution, wherein the transposome complexes in solution comprise a transposase and a transposon comprising a transposon end sequence, an RNA-specific barcode, and a sequence that hybridizes to capture probes, to prepare tagged fragments of the DNA:RNA duplexes, wherein the tagged fragments comprise the RNA-specific barcode and the sequence that hybridizes to capture probes; combining the sample with a second solid support having a surface comprising capture probes; and immobilizing the tagged fragments of DNA:RNA duplexes on the second solid support.

In some embodiments, the capture probes comprise nucleic acids.

### EXAMPLES

### Example 1

### Preparation of an RNA Sequencing Library Using BLTs Comprising Capture Oligonucleotides

An RNA sequencing library can be prepared from a full-length total RNA from a sample comprising RNA using methods described herein.

The mRNA from a sample can be immobilized to RNA bead-linked transposomes (BLTs) by binding of the polyA tails of the mRNA to the polyT capture oligonucleotides on the beads.

Then, a reverse transcriptase is used for cDNA synthesis. The reverse transcriptase polymerase is used to generate a DNA:RNA duplex from the target RNA bound to the bead. Exemplary reagents for cDNA synthesis include a reverse transcriptase, random primers, oligo dT primers, dNTPs and/or an Rnase inhibitor. Both random primers and oligo dT primers may be used in a cDNA synthesis reaction.

The cDNA synthesis reaction may be run at 42° C. for 90 minutes and then 85° C. for 5 minutes. The sample does not need to be washed after cDNA synthesis.

Tagmentation of the DNA:RNA duplexes can then be performed with RNA BLTs. A variety of BLTs have been described that can be used to generate RNA BLTs. The tagmentation of the DNA:RNA duplexes serves to generate DNA:RNA fragments that are immobilized to the beads by the transposomes.

The transposome complexes of the BLTs may comprise a transposase bound to a first polynucleotide comprising a 3′ portion comprising a transposon end sequence and a first tag. In this way, the first tag is incorporated during generation of the DNA:RNA fragments.

After fragmentation, strand exchange and gap-fill ligation are performed. In some embodiments, a second tagmentation reaction is performed to generate double-stranded DNA fragments wherein one end is in solution. The second tagmentation reaction may incorporate a second tag.

The library can then be released for further methods that can be performed in- tube or in-flowcell. This methodology allows methods that provide sequence of the full length of mRNAs.

Such methods may also be used with activatable BLTs, that comprise an immobilized oligonucleotide that can bind to transposomes in solution. Such a bead is shown in FIG. 13, wherein an A′ sequence in a second transposon can bind to a shortA sequence in an immobilized polynucleotide used to bind transposomes. Immobilized oligonucleotides may also comprise an adapter (such as a P5 sequence) and a bead code (BC), as shown in FIG. 13.

### Example 2

### Preparation of Multiomic Sequencing Libraries Using 3 Beads

An approach using 3 beads can be used to generate RNA and DNA sequencing libraries from a sample comprising RNA and DNA, as shown in FIG. 24. This method utilizes RNA capture beads that can bind RNA but do not have transposomes.

In this method, a DNA BLT may be used for DNA tagmentation while the RNA is captured by the RNA capture beads. After washing, the RNA is transferred from the RNA capture beads to the RNA BLTs. After reverse transcription, the DNA:RNA duplexes can be fragmented and tagged by the active RNA BLTs. After strand exchange and gap-fill ligation, the RNA and DNA sequencing libraries can then be released from the respective BLTs.

### Example 3

### Preparation of Multiomic Sequencing Libraries Using DNA BLTs and RNA Beads Lacking Active Transposome Complexes

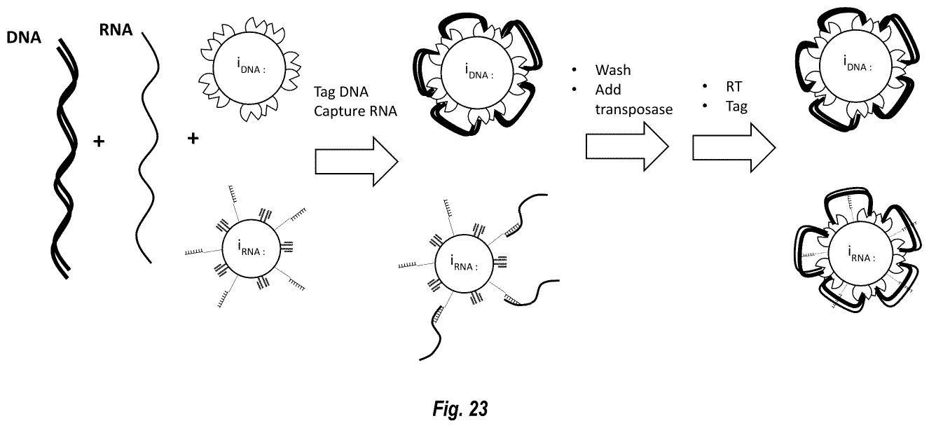

DNA BLTs and RNA BLTs lacking transposases, followed by addition of transposases to generate RNA BLTs, can be used to generate RNA and DNA sequencing libraries from a sample comprising RNA and DNA, as shown in FIG. 23. BLTs that bind RNA but lack transposases, may be referred to as “naked” RNA BLTs.

In this method, a DNA BLT may be used to tagment DNA while the RNA is captured by the naked RNA BLTs. After washing, transposases are added to activate the RNA BLTs. After reverse transcription, the DNA:RNA duplexes can be tagmented by the active RNA BLTs. After strand exchange and gap-fill ligation, the RNA and DNA sequencing libraries can then be released from the respective BLTs.

### Example 4

### Preparation of Multiomic Sequencing Libraries Using DNA BLTs and Deactivated RNA BL Ts

DNA BLTs and reversibly “deactivated” RNA BLTs can be used to generate RNA and DNA sequencing libraries from a sample comprising RNA and DNA, as shown in FIG. 22.

In this method, a DNA BLT may be used to tagment DNA while the RNA is captured by the deactivated RNA BLT. After washing, the deactivation of the RNA BLTs is reversed (i.e., the transposome complexes of the RNA BLTs are activated). After activation, reverse transcription is performed and the DNA:RNA duplexes can be fragmented and tagged by the active RNA BLTs. After strand exchange and gap-fill ligation, the RNA and DNA sequencing libraries can then be released from the respective BLTs.

A variety of reversible transposome deactivators are known, such as dephosphorylated ME′, extra cleavable bases on the transposon end, inhibitor duplexes can bind to transposomes, and heat-labile antibodies that can complex to the DNA binding site of the transposase.

### Example 5

### Preparation of Multiomic Sequencing Libraries Using 2 Beads With Sequential Immobilization of DNA and RNA

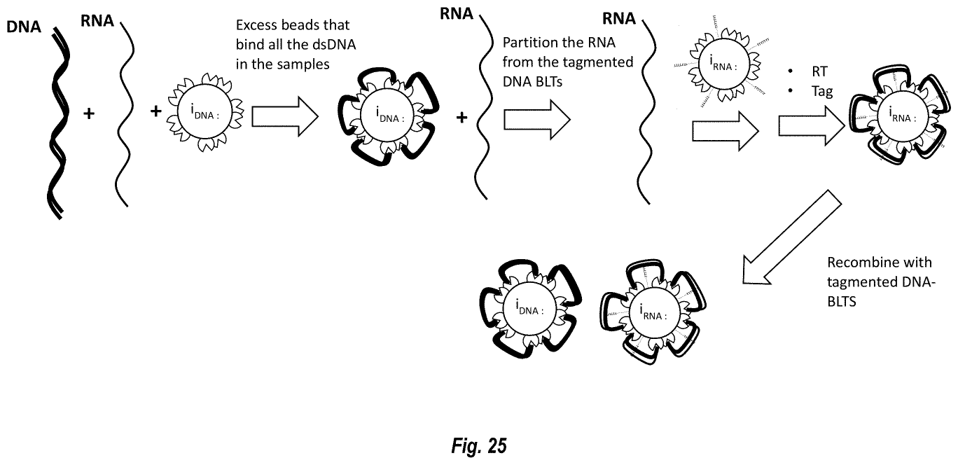

Sequential steps can be used to generate RNA and DNA sequencing libraries from a sample comprising RNA and DNA, as shown in FIG. 25. The DNA can be captured by DNA BLTs, and the DNA is tagmented. This step is done with an excess of beads, such that all of the double-stranded DNA is bound to the DNA BLTs. The RNA is then partitioned (i.e., physically separated or segregated) from the tagmented DNA BLTs. RNA is then captured by RNA BLTs comprising polyT capture oligonucleotides, and RNA-DNA duplexes are generated by a reverse transcriptase polymerase. The DNA:RNA duplexes can be tagmented by the transposome complexes of the RNA BLTs. After strand exchange and gap-fill ligation, the RNA and DNA sequencing libraries can then be released from the respective BLTs.

### Example 6

### Preparation of an RNA Sequencing Library via cDNA Synthesis in Solution

In some methods, a DNA:RNA duplex is generated in solution prior to binding to a BLT. In this method, the BLT may comprise capture oligonucleotides that bind to the DNA:RNA duplexes. Alternatively, the DNA:RNA duplexes can be bound by the transposases of the transposome complexes.

A sample comprising target RNA may be used to generate cDNA in solution. An exemplary solution for generating full-length cDNA may comprise a reverse transcriptase, primers, dNTPs and/or an Rnase inhibitor. An oligo dT primer can be used to prepare full-length cDNA from mRNA. An oligo dT primer and random primers can be used to prepare full-length total RNA. The cDNA synthesis can be performed at 42° C. for 90 minutes followed by 85° C. for 5 minutes without washing. In this way, DNA:RNA duplexes are generated in solution (as shown in FIG. 2).

These DNA:RNA duplexes can be bound to BLTs for tagmentation. This tagmentation can be performed with standard Illumina DNA Flex PCR-Free (research use only, RUO) technology, previously known as Illumina's Nextera technology, that uses two different transposome complexes to incorporate different adapter sequences at opposite ends of fragments. Alternatively a method using BLTs for symmetric tagmentation, followed by primer extension to introduce another adapter, may be used.

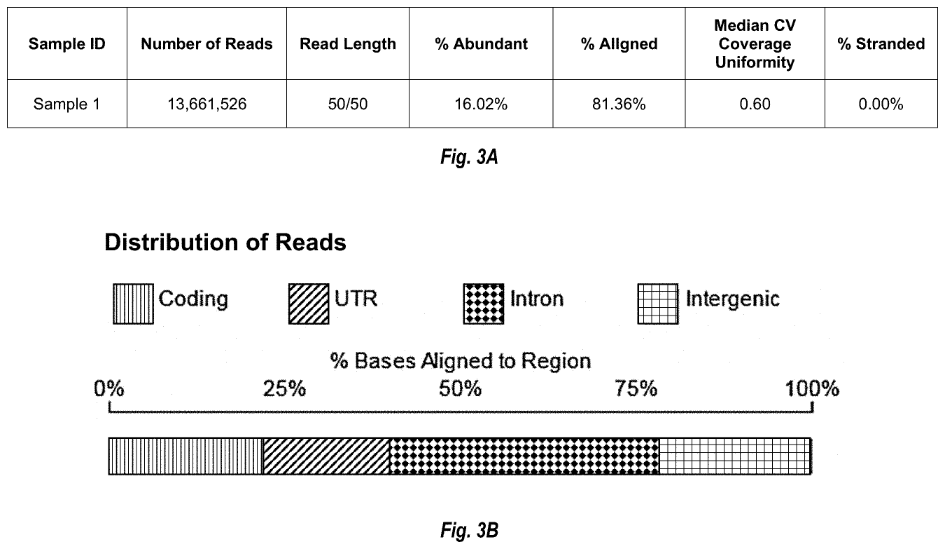

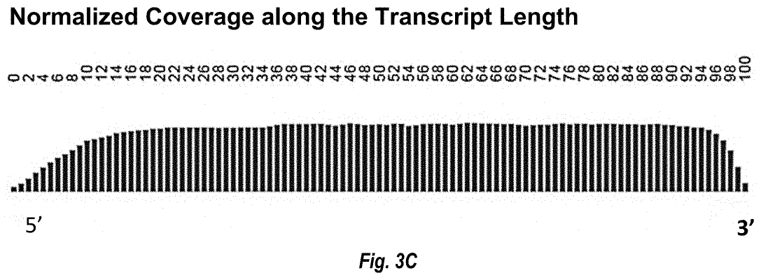

After generating the immobilized DNA:RNA fragments, the transposase may be stopped or removed (such as by SDS). Strand exchange can generate double-stranded DNA, followed by gap filling and ligation. Then, the prepared library can be released. The method may be performed in a tube or flowcell. FIGS. 3A-3C show representative sequencing results from an RNA sequencing library generated using cDNA synthesis in solution from universal human reference RNA to generate DNA:RNA duplexes, following by fragmentation by beads comprising immobilized transposome complexes. A library was successfully generated from transcripts and aligns with exon sequences. These data support a relative lack of 3′ bias in the sequencing results when using a method including generation of DNA:RNA duplexes in solution, as the sequence reads represent the entire length of the original transcript molecule. Automated workflows using this method can generate sequencable libraries from total RNA with minimal user input.

FIG. 9 shows how polyT primers and preparation of DNA:RNA duplexes in solution with tagmentation and library preparation on BLTs can be used to prepare libraries from full-length mRNA. FIG. 10 shows how randomer primers and preparation of DNA:RNA duplexes in solution with tagmentation and library preparation on BLTs can be used to prepare libraries from total RNA. For both methods, libraries can be prepared on BLTs and released in a flowcell or tube. Such release in a flowcell can allow for release in a spatially localized manner, such that fragments from the same full-length RNA or mRNA will be released in close proximity on a flowcell, as these fragments were prepared on the same bead.

### Example 7

### Preparation of Multiomic Sequencing Libraries Using Dual RNA/DNA Barcoding and Partitioning

A method can generate bead-linked transposomes (BLTs) with different oligonucleotide index sequences, which are identifiable in NGS-reads (See Zhang et al., Nat. Biotech. 35: 852-857 (2017)). BLTs with different oligonucleotide indexes are applied during the library prep workflow to incorporate differential molecular tags used to identify RNA- and DNA-originating NGS reads.

A representative method is shown in FIG. 26. First, BLTs loaded with oligonucleotides index(es) identifying DNA target material (DNA-specific barcode') is applied to the total nucleic acid (TNA) sample. The double-stranded DNA (dsDNA) in the sample are specifically tagmented by the DNA-barcode BLTs (DNA BLTs). Magnetic stand separation can be applied to partition the tagmented DNA fragments on the DNA BLTs away from the RNA molecules in solution. The separated RNA molecules are then fully converted to ds-complementary DNA (ds-cDNA) by reverse transcription and second strand synthesis methods known by one of ordinary skill in the art, including ‘stranded’ and ‘non-stranded’ synthesis methods. BLTs with an RNA-identifying barcode (RNA-specific barcode' on an RNA BLT) are then applied to tagment the ds-cDNA sample.

The two tagmentation libraries can then be combined and amplified together or separately. The latter may be advantageous for specifying different amplification levels of RNA- and DNA-originating library molecules. Alternatively, the RNA-specific barcode and DNA-specific barcode may also comprise different primer binding sequences, that can enable differential RNA and DNA library amplification through different PCR conditions (e.g., different amplification cycle numbers for the RNA- and DNA amplification primer set). If RNA- and DNA-libraries are amplified separately, the PCR sample index may be used instead of indexed transposome to identify a NGS read as having RNA- or DNA-molecule origination.

After amplification, RNA- and DNA-libraries can be sequenced directly or enriched for targeted regions prior to sequencing. As either the RNA-specific barcode or the DNA-specific barcode is sequenced with every library fragment, these barcodes can be used for sorting those samples originating from DNA from those originating from RNA during secondary bioinformatic analysis.

### Example 8

### Preparation of Multiomic Sequencing Libraries Using BLT Tagmentation and Dual RNA/DNA Barcoding in a Single Reaction Vessel

Preparation of multiomic sequencing libraries can also be performed in a single reaction vessel (i.e., a single pot scheme) without partitioning.

DNA-specific barcode BLT (DNA BLT) tagmentation is performed on a TNA sample as in Example 7. After the DNA tagmentation reaction is complete, a ‘suicide dsDNA’ substrate comprising synthetic dsDNA can be added. The purpose of the synthetic dsDNA is to saturate the DNA-barcode BLT (DNA BLT) and to occupy (and be tagmented by) any remaining, unreacted DNA BLTs. This prevents unreacted DNA BLTs from cross-reacting with RNA- originating substrates in downstream steps. An exemplary suicide is uracil-containing dsDNA, which is a substrate for tagmentation but which will not be amplified in PCR reactions employing uracil-intolerant DNA polymerases.

In the same reaction tube, after dsDNA tagmentation, ds-cDNA is generated from the RNA molecules in solution. This cDNA synthesis may be achieved in two steps to generate ‘stranded libraries’ (e.g. employing second strand synthesis with uracil bases, with a similar effect as ‘suicide dsDNA’) or in one step using a template switch oligonucleotide and a compatible reverse transcriptase (for example, SMRT-seq, Takara Bio). Once ds-cDNA is generated, the RNA-specific barcode BLT (RNA BLT) is applied to specifically tagment these substrate molecules. The two tagmentation products are cleaned up and eluted together (in same tube). Differential primer binding sequences incorporated during tagmentation on the DNA BLTs versus RNA BLTs may also enable differential RNA- and DNA-library amplification levels. Optional enrichment, sequencing, and bioinformatic deconvolution of RNA- and DNA-originating molecules can be carried out as in Example 7.

### Example 9

### BLT Tagmentation with Dual RNA/DNA Barcoding using a Transposase with Activity on DNA:RNA Duplexes

The method described in Example 8 can be modified for use with DNA:RNA duplexes. This method obviates the need for synthesis of a second strand of cDNA (and associated cleanup) steps. After tagmentation of DNA to incorporate a DNA-specific barcode using DNA BLTs, reverse transcription is performed to generate first strand cDNA resulting in DNA:RNA duplexes. These DNA:RNA duplexes can be tagmented using an RNA-specific barcode BLT (RNA BLT) that has activity on DNA:RNA duplexes. XGEN has developed the ted-transposon product with activity on DNA:RNA duplexes. This method obviates a need to generate ds-cDNA.

### Example 10

### Preparation of RNA Sequencing Library with Single-Cell Resolution

BLTs can prepare libraries from transcripts in a single-cell format, using methods such as droplets or flowcells with microwells.

In methods that use droplets (FIGS. 4 and 5), cells are first isolated into droplets that contain at least one bead decorated with P5, a bead code (barcode), and oligo dT sequence. Approximately 1000 different types of barcode beads can be used, since spatial barcoding provides another 3 orders of magnitude for barcoding. Beads containing the hybridized mRNA to the beads are removed from droplets and cDNA synthesis performed using reverse transcriptase enzyme and nucleotides in bulk.

The term “in bulk” refers to cDNA synthesis occurring on beads that are in solution, so the beads are not in droplets, but the mRNA remains hybridized to beads and is not in solution itself. Thus, preparation of cDNA in bulk allows for a simpler workflow outside beads, but this preparation maintains spatial separation of different mRNAs on different beads. The library was generated with single-sided tagmentation with a transposon comprising a P7-ME sequence and completed using ligation.

Beads are delivered to the flowcell without release of fragments, and single cell RNA libraries are released on flowcell in spatially localized manner to achieve spatial barcoding. In this way, transcripts originating from a single cell can be resolved from transcripts from other cells, as transcripts from the same cell are in closer proximity. As shown in FIG. 5, this results in a library biased towards the 3′ end of the original mRNA molecules.

For workflows to prepare libraries from full-length mRNA (FIG. 7), the beads consist of two different types of oligonucleotides, polyT capture oligonucleotides to hybridize the mRNA and immobilized P5-barcode-shortA oligonucleotides to assemble the transposons. This shortA sequence can hybridize to a sequence in a transposon. In other words, these methods can use “activatable transposons” as described herein that can be used to assemble transposons.

After cDNA synthesis on the beads, the transposons are assembled on the beads using A5′ handle that is attached to the ME′ sequence of the second transposon. The long transcript loops around the bead and is tagmented at multiple sites. After this step, the ME′-A5′ (i.e., second transposon) is dehybridized, and a ME′-A5′-P7 oligonucleotide is hybridized and followed by gap-fill ligation reaction. This results in full length transcript generated from the mRNA molecule converted into linked-reads wrapped around the beads. Further, library fragments comprise one or more adapter incorporated during tagmentation and one or more adapter incorporated during ligation of the oligonucleotide. The library fragments are then released on the flowcell in spatially localized manner.

For performing a single cell workflow directly on flowcell, without requiring droplets, flowcells with microwells integrated can be used (FIG. 8). Beads as described above for droplets can be used, except with much fewer to no barcodes due to compartmentalization from microwells. In other words, the spatial resolution afforded by using microwell may obviate the needs for use of bead codes. Cells are captured inside the microwells (generally one cell per microwell). The mRNA from lysed cells is captured on beads within the microwells, and then converted to libraries using the approaches described above for droplet embodiments. Spatial resolution is afforded by the microwells, such that libraries are generally generated from a single cell within a given microwell.

FIGS. 11A-11C show some features of beads that may be used for such methods. FIG. 11A show beads after generation of a first strand of cDNA to prepare a DNA:RNA duplex, FIG. 11B shows how multiple transposomes can fragment the DNA:RNA duplex at multiple positions, and FIG. 11C shows beads with multiple capture oligonucleotides and assembled transposomes.

### Example 10

### Preparation of RNA Sequencing Library with 3′ UMI

Methods can combine 3′ UMI tagging for accurate quantification and linked long read bead codes to enable a full-length counting assay for single cells.

For this method, a UMI sequence and a primer landing site is incorporated into a polyT primer used for first strand cDNA synthesis (FIG. 14). This primer landing site may be a P5 or P7 (or their complement) that allows for capture of resulting library fragments on a flowcell. The polyT primer may also comprise other sequences, such as a first-read or second-read sequencing adapter sequence.

The second strand of cDNA may be prepared such as Smart-Seq from Clonetech which would enable preamplification of cDNA prior to tagmentation making this approach compatible with single cells and ultra-low RNA inputs (FIG. 16).

Next, full length double-stranded cDNA with 3′ UMIs are tagmented with bead-linked transposomes with a common bead code, such that each bead has a unique code. This will result in a single tagmentation to capture the 3′ end fragment of the double-stranded cDNA, while other fragments undergo symmetrical tagmentation to incorporate the same adapter sequences at both ends of fragments (FIG. 14). Next, second transposons are dehybridized and a polynucleotide is annealed to the ME sequence of the first transposome. The polynucleotide can comprise different adapter sequences than those introduced during tagmentation, to allow preparation of sequencable fragments for standard platforms. The “y-adaptor style” of hybridizing a polynucleotide after tagmentation on a bead-linked transposome makes it compatible with stranded RNA library preparations. This workflow may be more sensitive than standard asymmetric tagmentation workflows, which can lead to non-productive tagmentation events (wherein end fragments have the same adapters at both ends of fragments and cannot be sequenced on standard platforms).

Tagmentation may be performed with activatable BLTs, wherein a sequence within an immobilized oligonucleotide on the surface of the bead can hybridize to the second transposon (Hyb′-ME′) to assemble active transposomes (FIG. 14) or with BLTs wherein the first transposon (i.e., a first polynucleotide) of transposomes is immobilized to the bead (FIGS. 15 and 16).

Generally using this method, a full-length transcript will be tagmented by a single bead, which tagments all segments of the original full-length RNA with identical bead code sequences. This allows all fragments to be linked back to the original transcript as well as to the UMI introduced during reverse transcription (FIG. 15). In this way, fragments with the same bead code based on sequencing results, together with a UMI that marks unique transcripts (as opposed to duplicates or sequencing artifacts). This method quantitatively assesses full-length RNA and is compatible with 3′ UMI tagging, preamplification, and full-length RNA isoform detection.

### Example 11

### Preparation of RNA Sequencing Library via PRESS-BLT

A tagmentation reaction can be performed after a stranded method of cDNA preparation using a primer extension strand-specific BLT (PRESS-BLT) method. This workflow can create a strand-specific RNA-seq library using a BLT approach.

The first step of PRESS-BLT is cDNA synthesis. Like other strand-specific RNA sequencing approaches (such as TruSeq Stranded Total RNA®, Illumina) total RNA is copied into a first strand of cDNA using reverse transcriptase, random primers and a first strand synthesis buffer that includes Actinomycin D (for example, FSA buffer from Illumina). Actinomycin D specifically inhibits DNA-depended DNA synthesis and improves strand specificity.

Since double stranded cDNA is required for tagmentation and for stranded specificity, dTTP is replaced with dUTP in the second strand synthesis reaction. The incorporation of dUTP in the second strand suppresses its amplification in the index PCR during library preparation. This incorporation of dUTP in the second strand thus allows a strand-specific BLT approach.

Next, a BLT formulation is prepared. Transposomes are assembled by incubating the Tn5-V3 enzyme with an annealed double-stranded sequence that includes a 19bp mosaic end (ME) sequence. The top strand (i.e., first transposon) is termed the transfer strand as it is covalently attached to the 5′ end of the tagmented DNA or cDNA. The bottom strand (i.e., second transposon) is termed the non-transfer strand. After tagmentation, there is a 9bp gap between the non-transfer strand of the reverse complement ME sequence called the ME′ and the 3′ end of the tagmented DNA. For the primer extension workflow, at the 5′ end of the transfer strand there is a 14bp sequence called A14. A14 is one of the landing sites necessary for library amplification using index primers. Finally, attached to the 5′ end of the A14 sequence is a desthiobiotin modification. Desthiobiotin binds tightly to streptavidin and is used to attach the transposomes to the magnetic beads to form the BLTs. The desthiobiotin is used instead of biotin because it has a higher binding affinity to streptavidin compared to biotin. Use of desthiobiotin is important because it reduces carry-through of biotinylated DNA in the library product, which can affect post library prep enrichment workflows. Representative transposome complexes for use with PRESS-BLT are shown in FIG. 17.

Finally, strand-specific library preparation is performed by primer extension. With standard asymmetrical tagmentation methods in the art, cDNA is tagmented with BLTs that contain a mixture of A14 and B15 transposomes. Fragments that are tagmented with only A14 or only B15 do not make a viable library product. This means that roughly half of all tagmented fragments are lost leading to reduced library preparation efficiency.

The PRESS-BLT with primer extension workflow does not have this issue. cDNA is only tagmented with A14 transposomes (FIG. 17). The non-transferred ME′ is then melted off and gap-filled by PCR extension. A B15-ME primer is then annealed to the gap filled ME′ sequence and extended to make a copy that includes B15 on one end and the A14 on the other end of the insert. A UMI could be inserted in the primer between the B15 and ME or manufactured on the BLTs between the A14 and the ME sequences. UMIs are important as they mark unique mRNA transcripts and distinguish them from copies produced by PCR amplification. Following primer extension, the B15-insert-A14 copy is melted off the beads through heat or sodium hydroxide treatment (FIG. 18) and is transferred to a fresh tube for index primer amplification.

As shown in FIG. 19, a PRESS-BLT workflow may also use dual biotin for immobilizing transposomes, as this can improve binding and release of transposomes.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A method of preparing an immobilized library of tagged fragments from a sample comprising RNA and DNA, wherein the tagged fragments comprise either a DNA-specific barcode or an RNA-specific barcode, comprising:
a. combining a sample comprising RNA and DNA with a first solid support for immobilizing DNA, wherein the first solid support comprises transposome complexes immobilized thereon, wherein the transposome complexes comprise a transposase and a transposon comprising a transposon end sequence and a DNA-specific barcode;
b. immobilizing the DNA;
c. performing tagmentation on the first solid support to prepare tagged fragments comprising a DNA-specific barcode;
d. preparing double-stranded cDNA from the RNA;
e. combining the sample with a second solid support for immobilizing cDNA, wherein the second solid support comprises transposome complexes immobilized thereon, wherein the transposome complexes comprise a transposase and a transposon comprising a transposon end sequence and an RNA-specific barcode; and
f. immobilizing the cDNA and performing tagmentation on the second solid support to prepare tagged fragments comprising an RNA-specific barcode.

2. A method of preparing an immobilized library of tagged fragments from a sample comprising RNA and DNA, wherein the tagged fragments comprise either a DNA-specific barcode or an RNA-specific barcode, comprising:
a. combining a sample comprising RNA and DNA with a first solid support for immobilizing DNA, wherein the first solid support comprises transposome complexes immobilized thereon, wherein the transposome complexes comprise a transposase and a transposon comprising a transposon end sequence and a DNA-specific barcode;
b. immobilizing the DNA;
c. performing tagmentation on the first solid support to prepare tagged fragments comprising a DNA-specific barcode;
d. preparing a single strand of cDNA from the RNA to produce DNA:RNA duplexes;
e. combining the sample with a second solid support for immobilizing DNA:RNA duplexes, wherein the second solid support comprises transposome complexes immobilized thereon, wherein the transposome complexes comprise a transposase with activity on DNA:RNA duplexes and a transposon comprising a transposon end sequence and an RNA- specific barcode; and
f. immobilizing the DNA:RNA duplexes and performing tagmentation on the second solid support to prepare tagged fragments comprising an RNA-specific barcode.

3. A method of preparing an immobilized library of tagged fragments from a sample comprising RNA and DNA, wherein the tagged fragments comprise either a DNA-specific barcode or an RNA-specific barcode, comprising:
a. combining a sample comprising RNA and DNA with a first solid support for immobilizing DNA, wherein the first solid support comprises transposome complexes immobilized thereon, wherein the transposome complexes comprise a transposase and a transposon comprising a transposon end sequence and a DNA-specific barcode;
b. immobilizing the DNA;
c. performing tagmentation on the first solid support to prepare tagged fragments comprising a DNA-specific barcode;
d. preparing double-stranded cDNA from the RNA;
e. performing tagmentation on the double-stranded DNA in solution, wherein the transposome complexes in solution comprise a transposase and a transposon comprising a transposon end sequence, an RNA-specific barcode, and a sequence that hybridizes to capture probes, to prepare tagged fragments of the double-stranded cDNA, wherein the tagged fragments comprise the RNA-specific barcode and the sequence that hybridizes to capture probes;
f. combining the sample with a second solid support having a surface comprising capture probes; and
g. immobilizing the tagged fragments of double-stranded cDNA on the second solid support.

4. A method of preparing an immobilized library of tagged fragments from a sample comprising RNA and DNA, wherein the tagged fragments comprise either a DNA-specific barcode or an RNA-specific barcode, comprising:
a. combining a sample comprising RNA and DNA with a first solid support for immobilizing DNA, wherein the first solid support comprises transposome complexes immobilized thereon, wherein the transposome complexes comprise a transposase and a transposon comprising a transposon end sequence and a DNA-specific barcode;
b. immobilizing the DNA;
c. performing tagmentation on the first solid support to prepare tagged fragments comprising a DNA-specific barcode;
d. preparing a single strand of cDNA from the RNA to produce DNA:RNA duplexes;
e. performing tagmentation on the DNA:RNA duplexes in solution, wherein the transposome complexes in solution comprise a transposase and a transposon comprising a transposon end sequence, an RNA-specific barcode, and a sequence that hybridizes to capture probes, to prepare tagged fragments of the DNA:RNA duplexes, wherein the tagged fragments comprise the RNA-specific barcode and the sequence that hybridizes to capture probes;
f. combining the sample with a second solid support having a surface comprising capture probes; and
g. immobilizing the tagged fragments of DNA:RNA duplexes on the second solid support.

5. The method of claim 1, wherein the DNA-specific barcode and the RNA-specific barcode comprise different primer binding sequences, optionally wherein the method further comprises:
a. amplifying tagged fragments comprising the DNA-specific barcode using a primer that binds the primer binding sequence comprised in the DNA-specific barcode;
b. amplifying tagged fragments comprising the RNA-specific barcode using a primer that binds the primer binding sequence comprised in the RNA-specific barcode; or
c. amplifying tagged fragments comprising the DNA-specific barcode and tagged fragments comprising the RNA-specific barcode using a primer mix comprising a primer that binds the primer binding sequence comprised in the DNA-specific barcode and a primer that binds the primer binding sequence comprised in the RNA-specific barcode.

6. A method of preparing strand-specific libraries of single-stranded DNA from RNA comprising:
a. preparing a first strand of cDNA from an RNA comprised in a sample using a reverse transcriptase, a primer, and nucleotides comprising dTTP under conditions that inhibit DNA-dependent DNA synthesis;
b. preparing a second strand of cDNA from the first strand of cDNA using a DNA polymerase, a primer, and nucleotides comprising dUTP to prepare double-stranded cDNA;
c. applying the double-stranded cDNA to a solid support having transposome complexes immobilized thereon, wherein each transposome complex comprises:
i. a transposase;
ii. a first transposon comprising a 3′ portion comprising a transposon end sequence and a first-read sequencing adapter sequence; wherein the first transposon comprises a 5′ affinity element for immobilizing the transposome complex to the solid support; and
iii. a second transposon sequence comprising a sequence all or partially complementary to the transposon end sequence;
d. performing tagmentation on the double-stranded DNA with the transposome complexes to prepare tagged double-stranded DNA fragments comprising the first-read sequencing adapter sequence;
e. removing the second transposon and performing gap-filling and extension;
f. separating the strands of the double-stranded DNA fragments;
g. hybridizing a primer comprising a second-read sequencing adapter sequence to the transposon end sequence or the sequence all or partially complementary to the transposon end sequence and amplifying to prepare a DNA strand that is not attached to the solid support and that comprises the first-read sequencing adapter and the second-read sequencing adapter; and
h. releasing the strand generating by the amplifying from the solid support, wherein the releasing releases a single-stranded DNA fragment comprising the first-read sequencing adapter and the second-read sequencing adapter.

7. The method of claim 6, wherein:
a. the conditions that inhibit DNA-dependent DNA synthesis is the presence of a buffer comprising actinomycin D;
b. the primer is one or more randomer primers or the primer is a mix of a randomer primer and a polyT primer;
c. the primer for the preparing a second strand of cDNA is the same as the primer for the preparing a first strand of cDNA;
d. the RNA is a long non-coding RNA or antisense transcript;
e. the amplifying is performed with a uracil-intolerant polymerase; and/or
f. the affinity element is a biotin, desthiobiotin, or dual biotin, and the solid support comprises streptavidin or avidin on its surface.

8. A method of preparing a library of double-stranded DNA fragments from RNA comprising:
a. preparing a first strand of cDNA from a full-length RNA in a sample using a polyT primer comprising a UMI and a first-read sequencing adapter sequence;
b. preparing a second strand of cDNA to generate double-stranded cDNA;
c. applying the double-stranded cDNA to a bead having transposome complexes immobilized thereon, wherein each transposome complex comprises:
i. a transposase;
ii. a first transposon comprising a 3′ transposon end sequence; and
iii. a second transposon comprising a sequence all or partially complementary to
the transposon end sequence and a hybridization sequence;
wherein the transposome complex is immobilized by binding of the hybridization sequence to an oligonucleotide immobilized to a bead, wherein said oligonucleotide comprises a 5′ affinity element, a first-read sequencing adapter sequence, a bead code, and a sequence all or partially complementary to the hybridization sequence;
d. immobilizing the double-stranded cDNA and performing tagmentation on the bead to prepare double-stranded DNA fragments;
e. removing the second transposon;
f. hybridizing a primer comprising a second-read sequencing adapter sequence and a sequence all or partially complementary to the transposon end sequence to the transposon end sequence; and
g. performing gap-filling and extension to prepare double-stranded DNA fragments comprising the first-read sequencing adapter and the second-read sequencing adapter.

9. A method of preparing a library of double-stranded DNA fragments from RNA comprising:
a. preparing a first strand of cDNA from a full-length RNA in a sample using a polyT primer comprising a UMI and a first-read sequencing adapter sequence;
b. preparing a second strand of cDNA to generate double-stranded cDNA;
c. applying the double-stranded cDNA to a bead having transposome complexes immobilized thereon, wherein each transposome complex comprises:
i. a transposase;
ii. a first transposon comprising a 3′ transposon end sequence, a bead code, and a second-read sequencing adapter sequence; wherein the first transposon further comprises a 5′ affinity element for immobilizing the transposome complex to the solid support; and
iii. a second transposon comprising a sequence all or partially complementary to the transposon end sequence;
d. immobilizing the double-stranded cDNA and performing tagmentation on the bead to prepare double-stranded DNA fragments;
e. removing the second transposon;
f. hybridizing a primer comprising a second-read sequencing adapter sequence and a sequence all or partially complementary to the transposon end sequence to the transposon end sequence; and
g. performing gap-filling and extension to prepare double-stranded DNA fragments comprising the first-read sequencing adapter and the second-read sequencing adapter.

10. A method of preparing an immobilized library of tagged DNA:RNA fragments from target RNA comprising:
a. applying a sample comprising target RNA to a solid support having transposome complexes and capture oligonucleotides immobilized thereon, optionally wherein the target RNA comprises a sequence complementary to at least a portion of one or more of the capture oligonucleotides and/or the capture oligonucleotides comprise a polyT sequence, wherein the transposome complexes comprise a transposase bound to a first polynucleotide comprising:
i. a 3′ portion comprising a transposon end sequence, and
ii. a first tag;
wherein the sample is applied to the solid support under conditions wherein the 3′ end of the target RNA binds to the capture oligonucleotides;
b. adding a reverse transcriptase polymerase under conditions to synthesize cDNA and generate immobilized DNA:RNA duplexes on the capture oligonucleotides; and
c. performing tagmentation on the DNA:RNA duplexes with the transposome complexes under conditions wherein the DNA:RNA duplexes are tagged on the 5′ end of one strand, thereby producing an immobilized library of DNA:RNA fragments wherein at least one strand is 5′-tagged with the first tag.

11. A method of preparing an immobilized library of tagged DNA:RNA fragments from target RNA comprising:
a. applying a sample comprising target RNA to a solid support having capture oligonucleotides and a first polynucleotide immobilized thereon, wherein the first polynucleotide comprises:
i. a 3′ portion comprising a transposon end sequence, and
ii. a first tag;
wherein the sample is applied to the solid support under conditions wherein the 3′ end of the target RNA binds to the capture oligonucleotides;
b. adding a transposase under conditions wherein the transposase binds to the first polynucleotide to form a transposome complex;
c. adding a reverse transcriptase polymerase under conditions to synthesize cDNA and generate immobilized DNA:RNA duplexes on the capture oligonucleotides; and
d. performing tagmentation on the DNA:RNA duplexes with the transposome complexes under conditions wherein the DNA:RNA duplexes are tagged on the 5′ end of one strand, thereby producing an immobilized library of DNA:RNA fragments wherein at least one strand is 5′-tagged with the first tag.

12. A method of preparing an immobilized library of tagged DNA:RNA fragments from target RNA comprising:
a. applying a sample comprising target RNA to a solid support having capture oligonucleotides and a first polynucleotide immobilized thereon, wherein the first polynucleotide comprises:
i. a 3′ portion comprising a transposon end sequence, and
ii. a first tag;
wherein the sample is applied to the solid support under conditions wherein the 3′ end of the target RNA binds to the capture oligonucleotides;
b. adding a reverse transcriptase polymerase under conditions to synthesize cDNA and generate immobilized DNA:RNA duplexes on the capture oligonucleotides;
c. adding a transposase under conditions wherein the transposase binds to the first polynucleotide to form a transposome complex; and
d. performing tagmentation on the DNA:RNA duplexes with the transposome complexes under conditions wherein the DNA:RNA duplexes are tagged on the 5′ end of one strand, thereby producing an immobilized library of DNA:RNA fragments wherein at least one strand is 5′-tagged with the first tag.

13. A method of preparing an immobilized library of tagged DNA:RNA fragments from target RNA comprising:
a. applying a sample comprising target RNA to a solid support having capture oligonucleotides immobilized thereon, optionally wherein the RNA is mRNA, and the capture oligonucleotide comprises a polyT sequence;
b. adding a reverse transcriptase polymerase under conditions to synthesize cDNA and generate immobilized DNA:RNA duplexes on the capture oligonucleotides; and
c. performing tagmentation on the DNA:RNA duplexes with the transposome complexes in solution under conditions wherein the DNA:RNA duplexes are tagged on the 5′ end of one strand, thereby producing an immobilized library of DNA:RNA fragments wherein at least one strand is 5′-tagged with the first tag.

14. A solid support comprising capture oligonucleotides and a first polynucleotide immobilized thereon, wherein the first polynucleotide comprises:
a. a 3′ portion comprising a transposon end sequence, and
b. a first tag.

15. A solid support comprising capture oligonucleotides and an immobilized oligonucleotide, wherein the immobilized oligonucleotide comprises a sequence for hybridizing to a hybridization sequence comprised in a second transposon comprised in a transposome complex.

16. A method of preparing an immobilized library of tagged DNA:RNA fragments from a sample comprising RNA and DNA, comprising:
a. applying the sample comprising RNA and DNA to:
i. a first solid support for immobilizing DNA comprising first transposome complexes immobilized thereon, wherein the first transposome complexes comprise a transposase and a first polynucleotide comprising a 3′ portion comprising a transposon end sequence, and optionally a first tag; and
ii. a second solid support having first capture oligonucleotides immobilized thereon;
wherein the sample is applied to the mixture of first and second solid supports under conditions wherein the DNA binds to the first transposome complexes on the first solid support and is fragmented and optionally tagged, and the RNA binds to the first capture oligonucleotides on the second solid support;
b. transferring the RNA bound to the second solid support to a third solid support having second capture oligonucleotides that bind the transferred RNA and second transposome complexes immobilized thereon, wherein the second transposome complexes comprise a transposase and a second polynucleotide comprising a 3′ portion comprising a transposon end sequence, and a second tag;
c. adding a reverse transcriptase polymerase under conditions to synthesize cDNA and generate immobilized DNA:RNA duplexes on the second capture oligonucleotides; and
d. performing tagmentation on the DNA:RNA duplexes with the second transposome complexes under conditions wherein the DNA:RNA duplexes are tagged on the 5′ end of one strand, thereby producing an immobilized library of DNA:RNA fragments wherein at least one strand is 5′-tagged with the first tag.

17. A method of preparing an immobilized library of tagged DNA:RNA fragments from a sample comprising RNA and DNA, comprising:
a. applying a sample comprising RNA and DNA to:
i. a first solid support for immobilizing DNA comprising first transposome complexes immobilized thereon, wherein the first transposome complexes comprise a transposase and a first polynucleotide comprising a 3′ portion comprising a transposon end sequence, and optionally a first tag; and
ii. a second solid support having capture oligonucleotides and a second polynucleotide immobilized thereon, wherein the second polynucleotide comprises:
a 3′ portion comprising a transposon end sequence, and
a second tag;
wherein the sample is applied to the mixture of first and second solid supports under conditions wherein the DNA binds to the first transposome complexes on the first solid support and is fragmented and optionally tagged, and the RNA binds to the capture oligonucleotides on the second solid support;
b. adding a transposase under conditions wherein the transposase binds to the second polynucleotide to form transposome complexes on the second solid support
c. adding a reverse transcriptase polymerase under conditions to synthesize cDNA and generate immobilized DNA:RNA duplexes on the second capture oligonucleotides; and
d. performing tagmentation on the DNA:RNA duplexes with the second transposome complexes under conditions wherein the DNA:RNA duplexes are tagged on the 5′ end of one strand, thereby producing an immobilized library of DNA:RNA fragments wherein at least one strand is 5′-tagged with the first tag.

18. A method of preparing an immobilized library of tagged DNA:RNA fragments from a sample comprising RNA and DNA, comprising:
a. applying a sample comprising RNA and DNA to:
i. a first solid support for immobilizing DNA comprising first transposome complexes immobilized thereon, wherein the first transposome complexes comprise a transposase and a first polynucleotide comprising a 3′ portion comprising a transposon end sequence, and optionally a first tag; and
ii. a second solid support for immobilizing RNA having capture oligonucleotides and second transposome complexes that are reversibly deactivated immobilized thereon, wherein the transposome complexes comprise a transposase bound to a second polynucleotide comprising a 3′ portion comprising a transposon end sequence, and a second tag; wherein the sample is applied to the mixture of first and second solid supports under conditions wherein the DNA binds to the first transposome complexes on the first solid support and is fragmented and optionally tagged, and the RNA binds to the capture oligonucleotides on the second solid support;
b. adding a reverse transcriptase polymerase under conditions to synthesize cDNA and generate immobilized DNA:RNA duplexes on the second capture oligonucleotides;
c. activating the second transposome complexes; and
d. performing tagmentation on the DNA:RNA duplexes with the activated second transposome complexes under conditions wherein the DNA:RNA duplexes are tagged on the 5′ end of one strand, thereby producing an immobilized library of DNA:RNA fragments wherein at least one strand is 5′-tagged with the first tag.

19. A method of preparing an immobilized library of tagged DNA:RNA fragments from a sample comprising RNA and DNA, comprising:
a. applying a sample comprising RNA and DNA to a first solid support for immobilizing DNA comprising first transposome complexes immobilized thereon, wherein the first transposome complexes comprise a transposase and a first polynucleotide comprising a 3′ portion comprising a transposon end sequence, and optionally a first tag, and wherein the sample is applied under conditions wherein the DNA binds to the first transposome complexes on the first solid support and is fragmented and optionally tagged;
b. separating the first solid support with the bound DNA from the RNA;
c. applying the RNA to a second solid support for immobilizing RNA having capture oligonucleotides and second transposome complexes immobilized thereon, wherein the second transposome complexes comprise a transposase bound to a second polynucleotide comprising a 3′ portion comprising a transposon end sequence, and a second tag, and wherein the RNA is applied under conditions wherein the RNA binds to the capture oligonucleotides on the second solid support;
d. adding a reverse transcriptase polymerase under conditions to synthesize cDNA and generate immobilized DNA:RNA duplexes on the second capture oligonucleotides; and
e. performing tagmentation on the DNA:RNA duplexes with the activated second transposome complexes under conditions wherein the DNA:RNA duplexes are tagged on the 5′ end of one strand, thereby producing an immobilized library of DNA:RNA fragments wherein at least one strand is 5′-tagged with the first tag.

20. A method of preparing an immobilized library of tagged DNA:RNA fragments from target RNA comprising:
a. adding a reverse transcriptase polymerase to a sample comprising target RNA under conditions to synthesize cDNA and generate DNA:RNA duplexes;
b. immobilizing DNA:RNA duplexes to a solid support having transposome complexes immobilized thereon,
wherein the transposome complexes comprise a transposase bound to a first polynucleotide comprising:
i. a 3′ portion comprising a transposon end sequence, and
ii. a first tag;
wherein the sample is applied to the solid support under conditions wherein the DNA:RNA duplexes bind to capture oligonucleotides or transposases directly; and
c. performing tagmentation on the DNA:RNA duplexes with the transposome complexes under conditions wherein the DNA:RNA duplexes are tagged on the 5′ end of one strand, thereby producing an immobilized library of DNA:RNA fragments wherein at least one strand is 5′-tagged with the first tag.

21. A sequencing library prepared using the method of claim 1.
